(19) United States
(12) Patent Application Publication
(10) Pub. No.: US 2026/0325623 A1
(43) Pub. Date: Oct. 22, 2026

(54) OLIGONUCLEOTIDE DENDRON MOLECULAR VACCINES

(71) Applicant: NORTHWESTERN UNIVERSITY, Evanston, IL (US)

(72) Inventors: Chad A. Mirkin, Wilmette, IL (US); Max Everett Distler, Evanston, IL (US); John Cavaliere, Evanston, IL (US); Michelle Hope Teplensky, Evanston, IL (US); Michael Evangelopoulos, Evanston, IL (US)

(21) Appl. No.: 19/105,587

(22) PCT Filed: Aug. 23, 2023
(86) PCT No.: PCT/US2023/072750

§ 371 Date: Feb. 21, 2025

Related U.S. Application Data
(60) Provisional application No. 63/373,315, filed on Aug. 23, 2022.
(60) Provisional application No. 63/476,601, filed on Dec. 21, 2022.

(51) Int. Cl. A61K 39/215; A61K 39/00; A61P 37/04; C12N 15/113

(52) U.S. Cl. CPC A61K 39/215; A61K 39/0011; A61P 37/04; C12N 15/113; A61K 2039/53; A61K 2039/575; C12N 2310/11; C12N 2310/314; C12N 2310/321; C12N 2310/52

(57) ABSTRACT

The present disclosure is directed to oligonucleotide dendrons comprising an oligonucleotide stem linked to one or more oligonucleotide branches. The disclosure also provides methods of using the oligonucleotide dendrons.

### CROSS-REFERENCE TO RELATED APPLICATIONS

This application claims the priority benefit under 35 U.S.C. § 119(e) of U.S. Provisional Application No. 63/373,315, filed Aug. 23, 2022 and U.S. Provisional Application No. 63/476,601, filed Dec. 21, 2022, which are incorporated herein by reference in their entirety.

### STATEMENT OF GOVERNMENT INTEREST

This invention was made with government support under grant numbers FA8650-15-2-5518 awarded by the Air Force Research Laboratory and FA9550-17-1-0348 awarded by the Department of Defense, and CA257926 awarded by the National Institutes of Health. The government has certain rights in the invention.

### INCORPORATION BY REFERENCE OF MATERIAL SUBMITTED ELECTRONICALLY

The Sequence Listing, which is a part of the present disclosure, is submitted concurrently with the specification as an XML file. The name of the XML file containing the Sequence Listing is “2022-145—SeqListing.xml”, which was created on Aug. 23, 2023 and is 22,874 bytes in size. The subject matter of the Sequence Listing is incorporated herein in its entirety by reference. To the extent differences exist between information/description of sequences in the specification and information in the Sequence Listing, the specification is controlling.

### BACKGROUND

Immunotherapy has emerged as a powerful approach to treat cancer due to its ability to train the immune system to attack specific cancer cells and reduce off target effects.¹⁻⁵ Cancer vaccines function via the delivery of two components: an adjuvant (an immune system activator) and an antigen (an immune system target).^{6,7}

### SUMMARY

Nanomaterials are particularly attractive agents for the delivery of adjuvant and antigen components because they provide unprecedented control over vaccine chemistry and structure, and hence their properties and functions.^{3, 6-13} Indeed, the rational design of nanoscale therapeutics allows one to tune the biodistribution, codelivery, kinetics of component processing, and temporal degradation of a vaccine, all of which are critical factors that determine its efficacy.¹⁴⁻¹⁹ These findings are the foundation for rational vaccinology and underscore the importance of vaccine structure and architecture in dictating vaccine function.

Provided herein are oligonucleotide (e.g., DNA) dendrons that are molecularly well-defined and can be used to access nanoparticle-like properties as a result of the highly oriented, dense packing of oligonucleotides on the dendron branches.^{27,27, 28 28} These dendritic architectures are typically comprised of oligonucleotide stems (for example and without limitation, a single-stranded oligonucleotide stem) that branch into multiple oligonucleotide strands. These multivalent oligonucleotide constructs undergo rapid cellular uptake, are resistant to degradation, and can elicit an enhanced therapeutic effect of conjugated cargo.²⁷ Herein, it is provided that by utilizing the oligonucleotide dendron as the basis for a vaccine (e.g., a cancer vaccine), structure-function relationships could be probed in a molecularly-defined manner, providing novel insights into vaccine design and function.

Applications of the technology described herein include, but are not limited to:
Cancer immunotherapy
Infectious Disease Therapeutic
Gene Regulation Agent
Diagnostics Tool (in vitro, in vivo)
Imaging
Small molecule drug delivery
Protein replacement therapy
Peptide therapeutics

Advantages of the technology described herein include, but are not limited to:
Molecularly precise
Small size
Ease of synthesis
Biological compatibility
Chemical addressability
Structural modularity

Accordingly, the present disclosure provides the design, synthesis, and evaluation of oligonucleotide dendron-based molecular vaccines as, for example and without limitation, cancer immunotherapies.

In some aspects, the disclosure provides an oligonucleotide dendron comprising an oligonucleotide stem linked to a plurality of oligonucleotide branches through one or more of a doubler moiety, a trebler moiety, or a combination thereof. In some embodiments, the oligonucleotide dendron is a DNA dendron, a RNA dendron, a modified oligonucleotide dendron, or a combination thereof. In some embodiments, the oligonucleotide dendron comprises about 2 to about 27 oligonucleotide branches. In further embodiments, the oligonucleotide dendron comprises 6 oligonucleotide branches. In still further embodiments, the oligonucleotide dendron comprises 9 oligonucleotide branches. In some embodiments, the oligonucleotide stem comprises a homopolymeric nucleotide sequence. In some embodiments, the oligonucleotide stem comprises an immunostimulatory oligonucleotide. In further embodiments, one or more or all of the plurality of oligonucleotide branches comprises an immunostimulatory oligonucleotide. In still further embodiments, one or more or all of the plurality of oligonucleotide branches comprises a homopolymeric nucleotide sequence. In some embodiments, the immunostimulatory oligonucleotide is a toll-like receptor (TLR) agonist. In various embodiments, the TLR agonist is a toll-like receptor 1 (TLR-1) agonist, toll-like receptor 2 (TLR-2) agonist, toll-like receptor 3 (TLR-3) agonist, toll-like receptor 4 (TLR-4) agonist, toll-like receptor 5 (TLR-5) agonist, toll-like receptor 6 (TLR-6) agonist, toll-like receptor 7 (TLR-7) agonist, toll-like receptor 8 (TLR-8) agonist, toll-like receptor 9 (TLR-9) agonist, toll-like receptor 10 (TLR-10) agonist, toll-like receptor 11 (TLR-11) agonist, toll-like receptor 12 (TLR-12) agonist, toll-like receptor 13 (TLR-13) agonist, or a combination thereof. In some embodiments, the oligonucleotide stem comprises a toll-like receptor (TLR) antagonist. In some embodiments, one or more or all of the plurality of oligonucleotide branches comprises a toll-like receptor (TLR) antagonist. In various embodiments, the TLR-antagonist is a toll-like receptor 1 (TLR-1) antagonist, toll-like receptor 2 (TLR-2) antagonist, toll-like receptor 3 (TLR-3) antagonist, toll-like receptor 4 (TLR-4) antagonist, toll-like receptor 5 (TLR-5) antagonist, toll-like receptor 6 (TLR-6) antagonist, toll-like receptor 7 (TLR-7) antagonist, toll-like receptor 8 (TLR-8) antagonist, toll-like receptor 9 (TLR-9) antagonist, toll-like receptor 10 (TLR-10) antagonist, toll-like receptor 11 (TLR-11) antagonist, toll-like receptor 12 (TLR-12) antagonist, toll-like receptor 13 (TLR-13) antagonist, or a combination thereof. In some embodiments, one or more or all of the plurality of oligonucleotide branches are double stranded. In some embodiments, one or more or all of the plurality of oligonucleotide branches are single stranded. In further embodiments, the oligonucleotide stem is double stranded. In still further embodiments, the oligonucleotide stem is single stranded. In some embodiments, the oligonucleotide dendron comprises one or more additional agents. In further embodiments, the oligonucleotide dendron comprises 1, 2, 3, 4, 5, 6, 7, 8, 9, 10, 11, 12, 13, 14, 15, 16, 17, 18, 19, 20, 21, 22, 23, 24, 25, 26, 27 or more additional agents. In some embodiments, the oligonucleotide stem comprises an additional agent. In some embodiments, one or more or all of the plurality of oligonucleotide branches comprises an additional agent. In further embodiments, a free end of the oligonucleotide stem is conjugated to the additional agent. In some embodiments, the additional agent is conjugated to a midpoint or about a midpoint of the oligonucleotide stem. In some embodiments, a free end of one or more or all of the plurality of oligonucleotide branches is conjugated to the additional agent. In some embodiments, one or more or all of the plurality of oligonucleotide branches is conjugated to the additional agent at a midpoint or about a midpoint of the one or more or all of the plurality of oligonucleotide branches. In various embodiments, the additional agent is an oligonucleotide, a protein, a peptide, or a combination thereof. In some embodiments, the additional agent is an antigen. In further embodiments, the antigen is a viral antigen, a cancer-related antigen, or a combination thereof. In some embodiments, the oligonucleotide stem comprises an inhibitory oligonucleotide. In further embodiments, one or more or all of the plurality of oligonucleotide branches comprises an inhibitory oligonucleotide. In still further embodiments, the inhibitory oligonucleotide is antisense DNA, small interfering RNA (siRNA), an aptamer, a short hairpin RNA (shRNA), a DNAzyme, or an aptazyme. In various embodiments, the doubler moiety comprises a structure prior to incorporation into the oligonucleotide dendron that is:

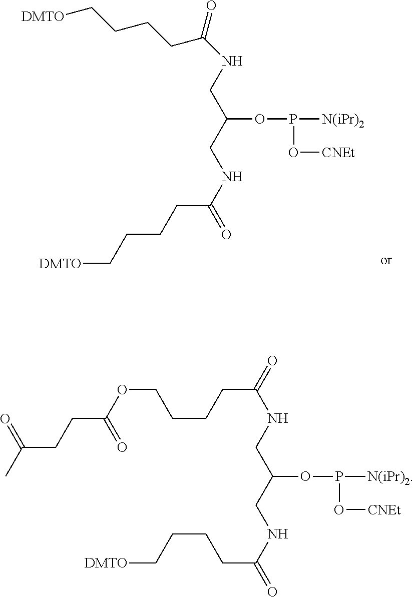

DMT=4,4′-dimethoxytriryl; O=oxygen; C=carbon; N=nitrogen; Et=ethyl; iPr=isopropyl. In some embodiments, the trebler moiety comprises a structure prior to incorporation into the oligonucleotide dendron that is:

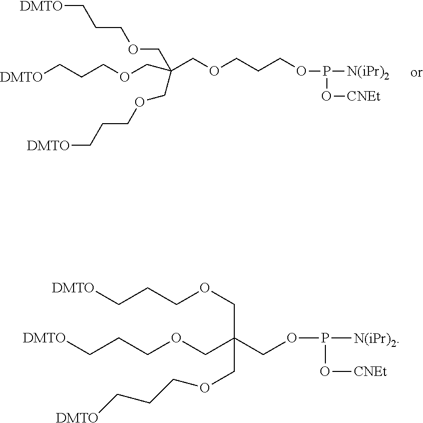

DMT=4,4′-dimethoxytriryl; O-oxygen; C-carbon; N=nitrogen; Et=ethyl; iPr=isopropyl.

In some aspects, the disclosure provides a pharmaceutical formulation comprising a plurality of the oligonucleotide dendrons of the disclosure and a pharmaceutically acceptable carrier or diluent.

In some aspects, the disclosure provides a vaccine comprising the oligonucleotide dendron or pharmaceutical formulation of the disclosure. In some embodiments, the vaccine comprises an adjuvant.

In some aspects, the disclosure provides an antigenic composition comprising an oligonucleotide dendron of the disclosure in a pharmaceutically acceptable carrier, diluent, stabilizer, preservative, or adjuvant, a pharmaceutical formulation of the disclosure, or a vaccine of the disclosure, wherein the antigenic composition is capable of generating an immune response including antibody generation, an antitumor response, an antiviral response, and/or a protective immune response in a mammalian subject. In some embodiments, the antibody response is a neutralizing antibody response or a protective antibody response.

In some aspects, the disclosure provides a method of producing an immune response in a subject in need thereof, comprising administering to the subject an effective amount of an oligonucleotide dendron, pharmaceutical formulation, vaccine, or antigenic composition of the disclosure, thereby producing the immune response in the subject. In some embodiments, the subject has cancer or is at risk of developing cancer, or the subject has a viral infection or is at risk of developing a viral infection, or a combination thereof. In various embodiments, the cancer is breast cancer, peritoneum cancer, cervical cancer, colon cancer, rectal cancer, esophageal cancer, eye cancer, liver cancer, pancreatic cancer, larynx cancer, lung cancer, skin cancer, ovarian cancer, prostate cancer, stomach cancer, testicular cancer, thyroid cancer, brain cancer, or a combination thereof. In some embodiments, the viral infection is due to a Coronaviridae virus, an Arteriviridae virus, a Roniviridae virus, a Picornaviridae virus, or a combination thereof. In further embodiments, the viral infection is due to Coronavirus, MERS, alphacoronavirus HCoV-NL63, betacoronaviruses HCoV-OC43, H1N1 influenza A, influenza BSARS, or a combination thereof. In still further embodiments, the Coronavirus is SARS-COV-2 and/or a variant thereof.

The disclosure also provides, in various aspects, a method of treating a cancer in a subject in need thereof, comprising administering to the subject an effective amount of the oligonucleotide dendron, pharmaceutical formulation, vaccine, or antigenic composition of the disclosure, thereby treating the cancer in the subject. In various embodiments, the cancer is breast cancer, peritoneum cancer, cervical cancer, colon cancer, rectal cancer, esophageal cancer, eye cancer, liver cancer, pancreatic cancer, larynx cancer, lung cancer, skin cancer, ovarian cancer, prostate cancer, stomach cancer, testicular cancer, thyroid cancer, brain cancer, or a combination thereof.

In further aspects, the disclosure provides a method of treating a viral infection in a subject in need thereof, comprising administering to the subject an effective amount of the oligonucleotide dendron, pharmaceutical formulation, vaccine, or antigenic composition of the disclosure, thereby treating the viral infection in the subject. In various embodiments, the viral infection is due to a Coronaviridae virus, an Arteriviridae virus, a Roniviridae virus, a Picornaviridae virus, or a combination thereof. In further embodiments, the viral infection is due to Coronavirus, MERS, alphacoronavirus HCoV-NL63, betacoronaviruses HCoV-OC43, H1N1 influenza A, influenza BSARS, or a combination thereof. In still further embodiments, the Coronavirus is SARS-COV-2 and/or a variant thereof. In some embodiments, the administering is subcutaneous. In various embodiments, the administering is intravenous, intraperitoneal, intranasal, or intramuscular.

In some aspects, the disclosure provides a composition comprising a plurality of the oligonucleotide dendrons of the disclosure.

In further aspects, the disclosure provides a method of inhibiting expression of a gene comprising the step of hybridizing a polynucleotide encoding the gene product with an oligonucleotide dendron or composition of the disclosure, wherein hybridizing between the polynucleotide and the oligonucleotide stem and/or one or more or all of the plurality of oligonucleotide branches of the oligonucleotide dendron occurs over a length of the polynucleotide with a degree of complementarity sufficient to inhibit expression of the gene product.

In some aspects, the disclosure provides a method for up-regulating activity of a toll-like receptor (TLR), comprising contacting a cell having the toll-like receptor with an oligonucleotide dendron or composition of the disclosure.

In further aspects, the disclosure provides a method for down-regulating activity of a toll-like receptor (TLR), comprising contacting a cell having the toll-like receptor with an oligonucleotide dendron or composition of the disclosure.

In some aspects, the disclosure provides an oligonucleotide dendron comprising an oligonucleotide stem linked to a plurality of oligonucleotide branches through one or more of a doubler moiety, a trebler moiety, or a combination thereof, wherein one or more of the plurality of oligonucleotide branches comprises an additional agent, and wherein the additional agent is conjugated at an internal location that is anywhere along the length of the one or more of the plurality of oligonucleotide branches that is not an end of the one or more of the plurality of oligonucleotide branches. In further aspects, the disclosure provides an oligonucleotide dendron comprising an oligonucleotide stem linked to a plurality of oligonucleotide branches through one or more of a doubler moiety, a trebler moiety, or a combination thereof, wherein a free end of the one or more of the plurality of oligonucleotide branches is conjugated to an additional agent. In some aspects, the disclosure provides an oligonucleotide dendron comprising an oligonucleotide stem linked to a plurality of oligonucleotide branches through one or more of a doubler moiety, a trebler moiety, or a combination thereof, wherein one or more of the plurality of oligonucleotide branches comprises an additional agent, and wherein (A) the additional agent is conjugated at an internal location that is anywhere along the length of the one or more of the plurality of oligonucleotide branches that is not an end of the one or more of the plurality of oligonucleotide branches, (B) a free end of the one or more of the plurality of oligonucleotide branches is conjugated to the additional agent, or a combination thereof. In some embodiments, the additional agent is conjugated at an internal location that is anywhere along the length of the one or more of the plurality of oligonucleotide branches that is not an end of the one or more of the plurality of oligonucleotide branches. In further embodiments, a free end of the one or more of the plurality of oligonucleotide branches is conjugated to the additional agent. In some embodiments, all of the plurality of oligonucleotide branches comprises an additional agent. In various embodiments, the oligonucleotide dendron is a DNA dendron, a RNA dendron, a modified oligonucleotide dendron, or a combination thereof. In further embodiments, the oligonucleotide dendron comprises about 2 to about 27 oligonucleotide branches. In some embodiments, the oligonucleotide dendron comprises 6 oligonucleotide branches. In some embodiments, the oligonucleotide dendron comprises 9 oligonucleotide branches. In further embodiments, the oligonucleotide stem comprises a homopolymeric nucleotide sequence. In some embodiments, the oligonucleotide stem comprises an immunostimulatory oligonucleotide. In various embodiments, one or more of the plurality of oligonucleotide branches comprises an immunostimulatory oligonucleotide. In some embodiments, all of the plurality of oligonucleotide branches comprises an immunostimulatory oligonucleotide. In further embodiments, one or more of the plurality of oligonucleotide branches comprises a homopolymeric nucleotide sequence. In still further embodiments, all of the plurality of oligonucleotide branches comprises a homopolymeric nucleotide sequence. In some embodiments, the immunostimulatory oligonucleotide is a toll-like receptor (TLR) agonist. In various embodiments, the TLR agonist is a toll-like receptor 1 (TLR-1) agonist, toll-like receptor 2 (TLR-2) agonist, toll-like receptor 3 (TLR-3) agonist, toll-like receptor 4 (TLR-4) agonist, toll-like receptor 5 (TLR-5) agonist, toll-like receptor 6 (TLR-6) agonist, toll-like receptor 7 (TLR-7) agonist, toll-like receptor 8 (TLR-8) agonist, toll-like receptor 9 (TLR-9) agonist, toll-like receptor 10 (TLR-10) agonist, toll-like receptor 11 (TLR-11) agonist, toll-like receptor 12 (TLR-12) agonist, toll-like receptor 13 (TLR-13) agonist, or a combination thereof. In some embodiments, the oligonucleotide stem comprises a toll-like receptor (TLR) antagonist. In further embodiments, one or more of the plurality of oligonucleotide branches comprises a toll-like receptor (TLR) antagonist. In still further embodiments, all of the plurality of oligonucleotide branches comprises a toll-like receptor (TLR) antagonist. In various embodiments, the TLR-antagonist is a toll-like receptor 1 (TLR-1) antagonist, toll-like receptor 2 (TLR-2) antagonist, toll-like receptor 3 (TLR-3) antagonist, toll-like receptor 4 (TLR-4) antagonist, toll-like receptor 5 (TLR-5) antagonist, toll-like receptor 6 (TLR-6) antagonist, toll-like receptor 7 (TLR-7) antagonist, toll-like receptor 8 (TLR-8) antagonist, toll-like receptor 9 (TLR-9) antagonist, toll-like receptor 10 (TLR-10) antagonist, toll-like receptor 11 (TLR-11) antagonist, toll-like receptor 12 (TLR-12) antagonist, toll-like receptor 13 (TLR-13) antagonist, or a combination thereof. In some embodiments, one or more of the plurality of oligonucleotide branches are double stranded. In further embodiments, all of the plurality of oligonucleotide branches are double stranded. In some embodiments, one or more of the plurality of oligonucleotide branches are single stranded. In further embodiments, all of the plurality of oligonucleotide branches are single stranded. In some embodiments, the oligonucleotide stem is double stranded. In some embodiments, the oligonucleotide stem is single stranded. In various embodiments, the oligonucleotide dendron comprises 1, 2, 3, 4, 5, 6, 7, 8, 9, 10, 11, 12, 13, 14, 15, 16, 17, 18, 19, 20, 21, 22, 23, 24, 25, 26, 27 or more additional agents. In various embodiments, the additional agent is a protein, a peptide, or a combination thereof. In some embodiments, the additional agent is an antigen. In further embodiments, the antigen is a viral antigen, a cancer-related antigen, or a combination thereof. In some embodiments, the oligonucleotide stem comprises an inhibitory oligonucleotide. In further embodiments, one or more of the plurality of oligonucleotide branches comprises an inhibitory oligonucleotide. In some embodiments, all of the plurality of oligonucleotide branches comprises an inhibitory oligonucleotide. In various embodiments, the inhibitory oligonucleotide is antisense DNA, small interfering RNA (siRNA), an aptamer, a short hairpin RNA (shRNA), a DNAzyme, or an aptazyme. In some embodiments, the doubler moiety comprises a structure prior to incorporation into the oligonucleotide dendron that is:

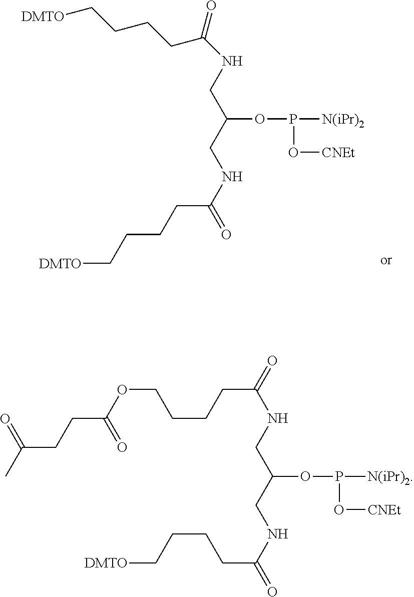

DMT=4,4′-dimethoxytriryl; O=oxygen; C=carbon; N=nitrogen; Et=ethyl; iPr=isopropyl. In some embodiments, the trebler moiety comprises a structure prior to incorporation into the oligonucleotide dendron that is:

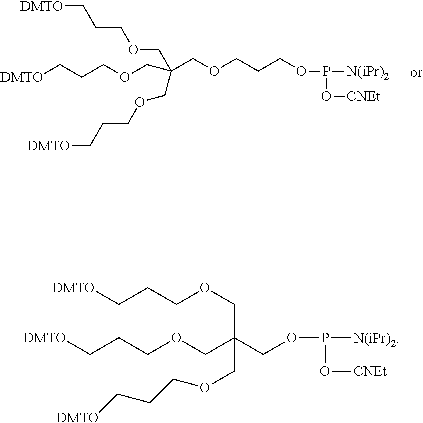

DMT=4,4′-dimethoxytriryl; O=oxygen; C=carbon; N=nitrogen; Et=ethyl; iPr=isopropyl. In some aspects, the disclosure provides an oligonucleotide dendron comprising an oligonucleotide stem linked to a plurality of oligonucleotide branches through one or more of a doubler moiety, a trebler moiety, or a combination thereof, wherein: (A) the oligonucleotide stem comprises an inhibitory oligonucleotide and is single-stranded, and (B) one or more of the plurality of oligonucleotide branches are single stranded. In some embodiments, all of the plurality of oligonucleotide branches are single stranded. In various embodiments, the inhibitory oligonucleotide is antisense DNA, small interfering RNA (siRNA), an aptamer, a short hairpin RNA (shRNA), a DNAzyme, or an aptazyme. In some embodiments, one or more or all of the plurality of oligonucleotide branches comprises a homopolymeric nucleotide sequence. In further embodiments, all of the plurality of oligonucleotide branches comprises a homopolymeric nucleotide sequence. In various embodiments, the oligonucleotide dendron is a DNA dendron, a RNA dendron, a modified oligonucleotide dendron, or a combination thereof. In some embodiments, the oligonucleotide dendron comprises about 2 to about 27 oligonucleotide branches. In some embodiments, the oligonucleotide dendron comprises 6 oligonucleotide branches. In some embodiments, the oligonucleotide dendron comprises 9 oligonucleotide branches. In some embodiments, the doubler moiety comprises a structure prior to incorporation into the oligonucleotide dendron that is:

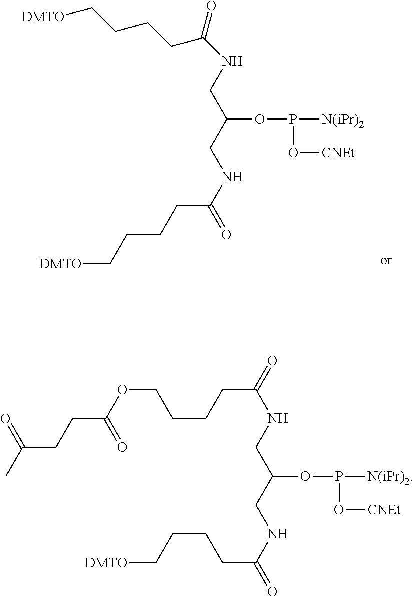

DMT=4,4′-dimethoxytriryl; O=oxygen; C=carbon; N=nitrogen; Et=ethyl; iPr=isopropyl. In some embodiments, the trebler moiety comprises a structure prior to incorporation into the oligonucleotide dendron that is:

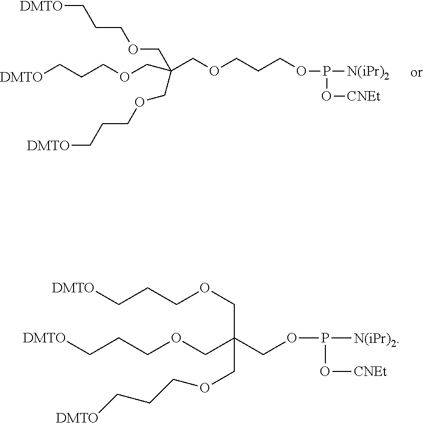

DMT=4,4′-dimethoxytriryl; O=oxygen; C=carbon; N=nitrogen; Et=ethyl; iPr=isopropyl. In further aspects, the disclosure provides a pharmaceutical formulation comprising a plurality of the oligonucleotide dendrons of the disclosure and a pharmaceutically acceptable carrier or diluent. In some aspects, the disclosure provides a vaccine comprising the oligonucleotide dendron or pharmaceutical formulation of the disclosure. In some embodiments, a vaccine of the disclosure further comprises an adjuvant. In some aspects, the disclosure provides an antigenic composition comprising an oligonucleotide dendron of the disclosure in a pharmaceutically acceptable carrier, diluent, stabilizer, preservative, or adjuvant, a pharmaceutical formulation or vaccine of the disclosure, wherein the antigenic composition is capable of generating an immune response including antibody generation, an antitumor response, an antiviral response, and/or a protective immune response in a mammalian subject. In some embodiments, the antibody response is a neutralizing antibody response or a protective antibody response. In some aspects, the disclosure provides a method of producing an immune response in a subject in need thereof, comprising administering to the subject an effective amount of an oligonucleotide dendron, a pharmaceutical formulation, a vaccine, or an antigenic composition of the disclosure, thereby producing the immune response in the subject. In some embodiments, the subject has cancer or is at risk of developing cancer, or the subject has a viral infection or is at risk of developing a viral infection, or a combination thereof. In further embodiments, the cancer is breast cancer, peritoneum cancer, cervical cancer, colon cancer, rectal cancer, esophageal cancer, eye cancer, liver cancer, pancreatic cancer, larynx cancer, lung cancer, skin cancer, ovarian cancer, prostate cancer, stomach cancer, testicular cancer, thyroid cancer, brain cancer, or a combination thereof. In still further embodiments, the viral infection is due to a Coronaviridae virus, an Arteriviridae virus, a Roniviridae virus, a Picornaviridae virus, or a combination thereof. In various embodiments, the viral infection is due to Coronavirus, MERS, alphacoronavirus HCoV-NL63, betacoronaviruses HCoV-OC43, H1N1 influenza A, influenza BSARS, or a combination thereof. In some embodiments, the Coronavirus is SARS-COV-2 and/or a variant thereof. In some aspects, the disclosure provides a method of treating a cancer in a subject in need thereof, comprising administering to the subject an effective amount of an oligonucleotide dendron, a pharmaceutical formulation, a vaccine, or an antigenic composition of the disclosure, thereby treating the cancer in the subject. In various embodiments, the cancer is breast cancer, peritoneum cancer, cervical cancer, colon cancer, rectal cancer, esophageal cancer, eye cancer, liver cancer, pancreatic cancer, larynx cancer, lung cancer, skin cancer, ovarian cancer, prostate cancer, stomach cancer, testicular cancer, thyroid cancer, brain cancer, or a combination thereof. In further aspects, the disclosure provides a method of treating a viral infection in a subject in need thereof, comprising administering to the subject an effective amount of an oligonucleotide dendron, a pharmaceutical formulation, a vaccine, or an antigenic composition of the disclosure, thereby treating the viral infection in the subject. In various embodiments, the viral infection is due to a Coronaviridae virus, an Arteriviridae virus, a Roniviridae virus, a Picornaviridae virus, or a combination thereof. In further embodiments, the viral infection is due to Coronavirus, MERS, alphacoronavirus HCoV-NL63, betacoronaviruses HCoV-OC43, H1N1 influenza A, influenza BSARS, or a combination thereof. In still further embodiments, the Coronavirus is SARS-COV-2 and/or a variant thereof. In some embodiments, the administering is subcutaneous. In some embodiments, the administering is intravenous, intraperitoneal, intranasal, or intramuscular. In some aspects, the disclosure provides a composition comprising a plurality of the oligonucleotide dendrons of the disclosure. In further aspects, the disclosure provides a method of inhibiting expression of a gene comprising the step of hybridizing a polynucleotide encoding the gene product with an oligonucleotide dendron or a composition of the disclosure, wherein hybridizing between the polynucleotide and the oligonucleotide stem and/or one or more or all of the plurality of oligonucleotide branches of the oligonucleotide dendron occurs over a length of the polynucleotide with a degree of complementarity sufficient to inhibit expression of the gene product. In some embodiments, hybridizing between the polynucleotide and the oligonucleotide stem and/or all of the plurality of oligonucleotide branches of the oligonucleotide dendron occurs over a length of the polynucleotide with a degree of complementarity sufficient to inhibit expression of the gene product. In further embodiments, the hybridizing between the polynucleotide and the oligonucleotide stem and/or one or more of the plurality of oligonucleotide branches of the oligonucleotide dendron occurs in the absence of a transfection reagent. In further embodiments, the hybridizing between the polynucleotide and the oligonucleotide stem and/or all of the plurality of oligonucleotide branches of the oligonucleotide dendron occurs in the absence of a transfection reagent. In some aspects, the disclosure provides a method for up-regulating activity of a toll-like receptor (TLR), comprising contacting a cell having the toll-like receptor with an oligonucleotide dendron or a composition of the disclosure. In further aspects, the disclosure provides a method for down-regulating activity of a toll-like receptor (TLR), comprising contacting a cell having the toll-like receptor with an oligonucleotide dendron or a composition of the disclosure.

### BRIEF DESCRIPTION OF THE DRAWINGS

FIG. 1 shows cellular uptake of immune stimulating DNA dendrons. (A) Four different DNA dendron designs were investigated: 1) 6 CpG sequences as the branches and a T10 stem (Dn6a); 2) 6 CpG sequences hybridized to the branches and a T10 stem (Dn6aH); 3) T10 branches and a CpG stem (Dn1a); 4) T10 branches and a CpG sequence hybridized to the stem (Dn1aH). All DNA dendrons have a cyanine 3 fluorescent dye in the middle of the molecule. T10 sequences were chosen due to synthetic ease and lack of secondary structure formation. (B) Cellular uptake after 1 h incubation at 250 nM shows the unhybridized structures have preferential access to cells. (C) Cellular uptake after 15 h incubation at 1 μM shows this trend holds, with hybridized branches having lower cellular uptake. The MFI+/−standard deviation (SD) is shown for n=3. ns=not significant; ***=p<0.001; ****=p<0.0001. Not all significances are shown for clarity.

FIG. 2 shows hybridized dendron designs, Dn1aH and Dn6aH, were formed by combining the DNA dendrons with the CpG strands at stoichiometric amounts and annealing from 90-20° C. over 1 h. By tuning the relative amount of CpG added, we determined that a 1:1 ratio of CpG complement to CpG strand can be used to form the full hybridized structures Dn6aH (left) and Dn1aH (right).

FIG. 3 shows dendron uptake with increasing branch length. Cells were treated with DNA dendrons that contained either T10 branches or T14 branches. Only at early timepoints (1 h) was it observed that longer branches resulted in increased cellular uptake at treatment concentrations of 50 and 250 nM.

FIG. 4 shows immune activation after treatment of murine bone marrow-derived dendritic cells with immune stimulating DNA dendrons. (A) Median fluorescence intensity (MFI) of CD86 (left) and CD80 (right) expression when the total dendron concentration was held constant. A similar trend was observed as that in the uptake experiment, whereby the unhybridized structures perform better than the hybridized constructs with no significant differences between Dn6a, Dn1a, and Dn1aH despite the 6-fold increase in CpG delivered by the Dn6a dendron. CpG 1 and CpG 6 represent CpG concentrations that correspond to Dn1a and Dn6a, respectively. (B) MFI of CD86 (left) and CD80 (right) expression when the total CpG concentration was held constant. Results indicated that the Dn1a dendron was the most efficient design to deliver functional DNA adjuvant. The MFI+/−standard deviation (SD) is shown for n=3. ns=not significant; ***=p<0.001; ****=p<0.0001. Not all significances are shown for clarity.

FIG. 5 shows the frequency of cells expressing CD86 and CD80 in response to treatment with the different dendron designs. These trends matched what was observed for the amount of CD86 and CD80 expression, with the greatest differences arising when CpG concentration was held constant.

FIG. 6 shows results of experiments in which dendritic cells were treated with either a CpG linear control, Dn1a with T10 branches, Dna with G-rich branches ((GGT) 4), or Dn1a with T14 branches, which matched the G-rich sequence in terms of the number of bases. After 15 h, it was observed that the G-rich branches had comparable uptake to the T10 branches, but resulted in a significant increase in DC activation (CD80 and CD86 expression).

FIG. 7 shows antigenic peptide conjugation and its impact on cellular uptake and immune activation. (A-C) MALDI-TOF MS of dendron-peptide conjugates, post purification, reveal expected mass shifts for each of the synthesized conjugates: Dn1E, Dn6Ee, and Dn6Em. (D) Cellular uptake of the dendron-peptide conjugates after 1 h at 250 nM. An increase in cellular uptake for the conjugates that contain a greater number of hydrophobic antigenic peptides is observed. (E) Immune activation of dendritic cells after 15 h treatment with dendron-peptide conjugates at 1 μM. The trends observed are similar to that seen in the cellular uptake experiments; the Dn6E structures produced a stronger immune response than did the Dn1E structure. The MFI+/−standard deviation (SD) is shown for n=3. ***=p<0.001; ****=p<0.0001. Not all significances are shown for clarity.

FIG. 8 shows results of experiments investigating Dn1, Dn6m, and Dn6e which are the dendrons that contain primary amines before peptide conjugation. It appeared that these amines impacted the cellular uptake of the dendron. Dn6m and Dn6e both have 6 primary amines and significantly higher cellular uptake than Dn1 that only has 1 primary amine. Furthermore, Dn6e, which has all of the amines on the 5′ terminal end of the dendron was taken up significantly more than the Dn6m, which had amines “buried” in the middle of the branches.

FIG. 9 shows that cells treated with the peptide conjugates showed increases in CD80 expression frequency and amount. These trends matched what was observed for the CD86 costimulatory marker in FIG. 7E.

FIG. 10 shows cells were treated with peptide-dendron conjugates after pretreatment with either fucoidan (scavenger receptor A inhibitor) or methyl-beta-cyclodextran (depletes lipid raft and cholesterol) or after incubation at 4° C. (active transport inhibition). Dendrons were significantly taken up by active transport, which is mostly facilitated by scavenger receptor A and partly by hydrophobic-mediated processes.

FIG. 11 shows ex vivo vaccine efficacy and in vivo vaccine uptake. (A) Murine PBMCs were treated with the dendron vaccines, raised T cells were then isolated and incubated in the presence of target TC-1 cancer cells at a ratio of 5:1, and cell death was quantified in terms of expression of both necrosis and early apoptosis markers (double positive, left) or early apoptosis markers (double and single positive, right). (B) Dendron vaccines were injected subcutaneously into female C57BL/6 mice (n=3) and fluorescence intensity of the lymph nodes was measured after 4 h, using IVIS. Results indicate that the Dn6Em structure was taken up significantly more than the Dn1E structure in vivo. (C) Representative scatter plots that correspond to the data reported in A. **=p<0.01; ***=p<0.001; ****=p<0.0001. Not all significances are shown for clarity.

FIG. 12 shows results of experiments in which female C57BL/6 mice (8-12 weeks old) were administered a single subcutaneous injection into the abdomen. The treatment dose was maintained at 6 nmol by Cy3 and dendron concentration. After 4 h, mice were euthanized, and the skin containing the lymph nodes was resected. Fluorescence was assessed using an IVIS 200 Spectrum (PerkinElmer) in vivo imaging system with a narrow band excitation of 535 nm and emission of 580 nm. Quantitative analysis was performed using Living Image software. The raw data presented here show that the Dn6Em vaccine was taken up by the lymph nodes more than the Dn1E vaccine.

FIG. 13 depicts treating tumor-bearing mice that were inoculated with cervical cancer cells. (A) Mice were treated with 2×10⁵ cancer cells on day 0 and received treatment once/week for four weeks, starting on day 7. Tumor measurements were taken every 2-3 days. (B) Tumor volumes at day 28, which demonstrate the significant tumor growth inhibition that results from Dn6Em treatment over the other groups. (C) Plots of the average tumor volumes show that the Dn6Em structure is the most effective vaccine for inhibiting tumor growth. (D) This inhibition in tumor growth led to a 100% survival rate for Dn6Em throughout the study (44 days). ns=not significant; *=p<0.05. Not all significances are shown for clarity. (E) Mice with no measurable tumors at the end of the study were re-challenged with TC-1 cancer cells. Mice that received the Dn6Em vaccine were protected against tumor growth.

FIG. 14 shows results of experiments in which female C57BL/6 mice aged 8-12 weeks (Jackson Laboratory) were inoculated with 2×10⁵ TC-1 tumor cells subcutaneously into the right flank, and they were allowed to grow to approximately 50 mm³ (7 days) prior to treatment. Treatments were administered at a dose of 60 UM in 100 μL volume by subcutaneous injection into the abdomen once per week, following the schedule provided. Tumor growth was measured every 2-3 days, and the volume was calculated using the following equation: tumor volume=length×width2×0.5. Animals were euthanized when tumor volumes reached 1,500 mm³ or when animal health necessitated sacrifice due to humane reasons. The spider plots of the individual mice in each group are presented.

FIG. 15 shows a schematic of oligonucleotide dendrons as molecularly precise therapeutics.

FIG. 16 shows a schematic of parameters that are used to elucidate structure-function relationships of molecular vaccines.

FIG. 17 shows that dendron structure directly affects vaccine efficacy in vivo.

FIG. 18 shows results of experiments demonstrating that oligonucleotide dendrons are agents for intracellular delivery.

FIG. 19 shows results of experiments in which the effect of E6 peptide conjugation on the DNA dendron's protein corona was studied by incubating Dn1a, Dn1E, and Dn6Em at 18.45 UM in either FBS (PBS, 10% FBS) or pure PBS at 37° C. for one hour with shaking. At the end of the one-hour incubation, sample corresponding to 0.03 OD (approximately 0.112 nmole) from each reaction was loaded on a 10% native PAGE gel and run at a constant voltage of 100 volts for one hour. Upon completion, images were obtained using the ChemiDoc Gel Scanner (BioRad) with the cyanine 3 filter.

FIG. 20 depicts the same data shown in FIG. 14 and provides further data from additional mice that were tested.

FIG. 21 depicts the gating strategy for FIG. 1B and FIG. 1C.

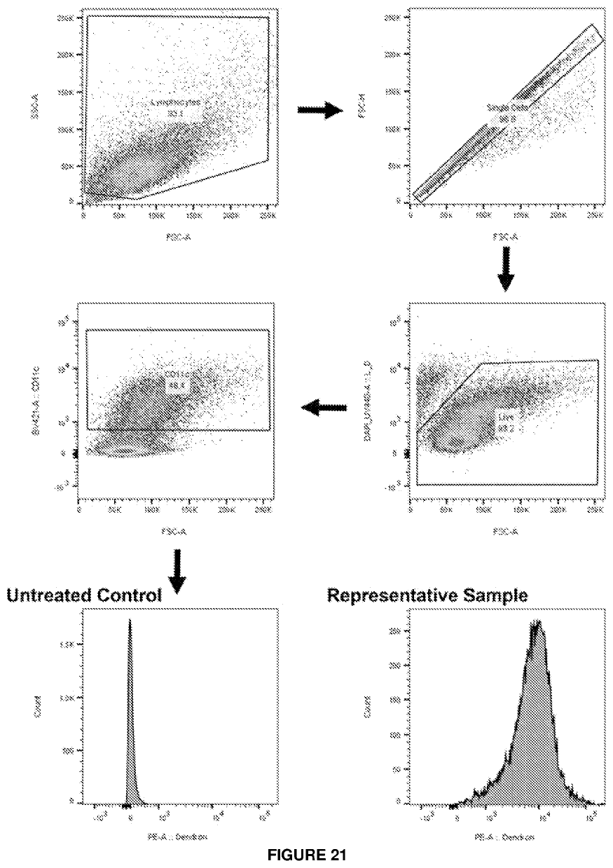

FIG. 22 depicts the gating strategy for FIG. 4A, FIG. 4B, FIG. 7D, and FIG. 7E.

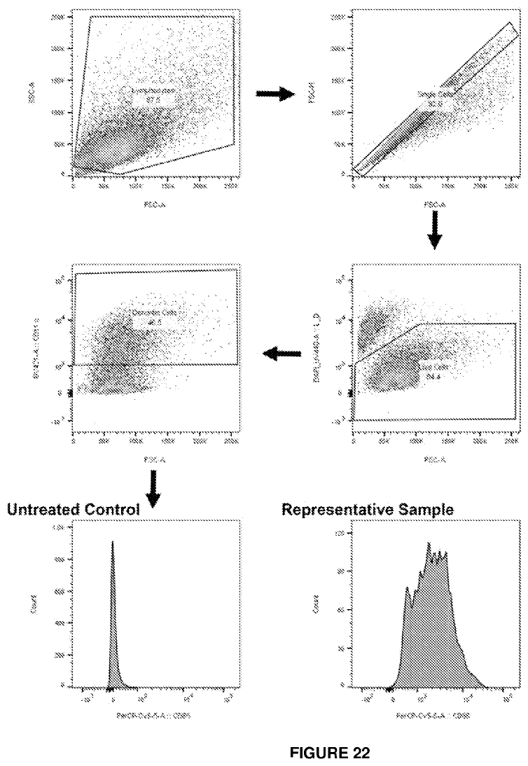

FIG. 23 depicts the gating strategy for FIG. 11A.

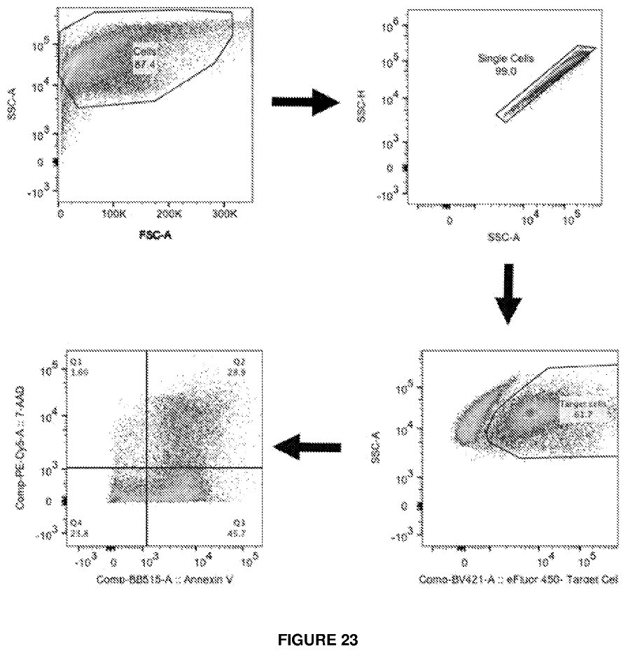

FIG. 24 shows a disulfide attachment that was incorporated using a heterobifunctional linker. Db=doubler, Tr=Trebler, Sp18=Spacer 18

FIG. 25 shows western blot results demonstrating that ASO dendrons with disulfide attachment chemistry enhance downregulation of STAT3.

FIG. 26 shows that ASO dendrons exhibited enhanced downregulation of STAT3.

### DETAILED DESCRIPTION

Vaccine structure plays a critical role in determining therapeutic efficacy, but in order to establish fundamental, effective, and translatable vaccine design parameters, a highly modular and well-defined platform is required. Dendritic oligonucleotide molecules, oligonucleotide strands that branch into a multivalent structure, are capable of highly efficient cellular uptake, resistance to degradation, and enhanced therapeutic efficacy. The oligonucleotide sequences that comprise the dendron can be designed to have specific functions (e.g., immunomodulatory or gene-regulatory) and specific valencies and lengths, which directly impact cellular uptake efficiencies. Furthermore, as described herein reactive chemical functional groups can be added to the oligonucleotide design enabling facile attachment of other therapeutic molecules (e.g., small molecules, RNA, peptides, and proteins). The conjugated cargo also is capable of enhanced cellular uptake and resistance to degradation, leading to a more potent therapeutic effect. The hydrophilicity of the oligonucleotide molecule improves the solubility of any conjugated cargo, therefore hydrophobic and hydrophilic cargo can be delivered using this approach. Taken together, the present disclosure provides routes to develop and deliver therapeutics with molecular precision. Herein, an oligonucleotide dendron vaccine that is capable of dendritic cell uptake, immune activation, and potent cancer killing is provided.

As used in this specification and the appended claims, the singular forms “a,” “an,” and “the” include plural reference unless the context clearly dictates otherwise.

The terms “polynucleotide” and “oligonucleotide” are interchangeable as used herein.

A “dendron” as used herein refers to an individual oligonucleotide molecule comprising an oligonucleotide stem linked to a plurality of oligonucleotide branches through one or more of a doubler moiety, a trebler moiety, or a combination thereof.

As used herein, an “oligonucleotide stem” is an oligonucleotide that is attached on one end to a doubler moiety, a trebler moiety, or a combination thereof, while the other end of the oligonucleotide stem is either free from attachment (i.e., a free end) or is attached to an additional agent as described herein. The oligonucleotide stem may be single stranded or double stranded. In some embodiments, the oligonucleotide stem of an oligonucleotide dendron is hybridized to an oligonucleotide, such as an oligonucleotide stem or oligonucleotide branch that is part of another oligonucleotide dendron.

As used herein, an “oligonucleotide branch” is an oligonucleotide that is connected on one end to an oligonucleotide stem through one or more doubler moieties, trebler moieties, or a combination thereof, while the other end of the oligonucleotide branch is either free from attachment (i.e., a free end) or is attached to an additional agent as described herein. An oligonucleotide branch may be single stranded or double stranded. An oligonucleotide branch may be attached to an additional agent as described herein. In some embodiments, one or more oligonucleotide branches of an oligonucleotide dendron is hybridized to an oligonucleotide such as an oligonucleotide stem or oligonucleotide branch that is part of another oligonucleotide dendron.

As used herein, the term “about,” when used to modify a particular value or range, generally means within 20 percent, e.g., within 10 percent, 5 percent, 4 percent, 3 percent, 2 percent, or 1 percent of the stated value or range.

Unless otherwise stated, all ranges contemplated herein include both endpoints and all numbers between the endpoints. The use of “about” or “approximately” in connection with a range applies to both ends of the range. Thus, “about 20 to 30” is intended to cover “about 20 to about 30”, inclusive of at least the specified endpoints.

A “subject” is a vertebrate organism. The subject can be a non-human mammal (e.g., a mouse, a rat, or a non-human primate), or the subject can be a human subject.

The terms “administering”, “administer”, “administration”, and the like, as used herein, refer to any mode of transferring, delivering, introducing, or transporting an oligonucleotide dendron to a subject in need of treatment with such an agent. Such modes include, but are not limited to, oral, topical, intravenous, intraarterial, intraperitoneal, intramuscular, intratumoral, intradermal, intranasal, and subcutaneous administration.

The term “vaccine” as used herein relates to an oligonucleotide dendron or a composition comprising an oligonucleotide dendron as described herein that upon administration induces an immune response, for example an antitumor response and/or a cellular immune response, which recognizes and attacks an antigen such as a cancer-related antigen. A vaccine may be used for the prevention, amelioration, or treatment of a disease (e.g., cancer, a viral infection).

As used herein, “treating” and “treatment” refers to any reduction in the severity and/or onset of symptoms associated with a disease or disorder (e.g., cancer, a viral infection). Accordingly, “treating” and “treatment” includes therapeutic and prophylactic measures. One of ordinary skill in the art will appreciate that any degree of protection from, or amelioration of, the disease (e.g., cancer, a viral infection) is beneficial to a subject, such as a human patient. The quality of life of a patient is improved by reducing to any degree the severity of symptoms in a subject and/or delaying the appearance of symptoms.

As used herein, an “immunostimulatory oligonucleotide” is an oligonucleotide that can stimulate (e.g., induce or enhance) an immune response. Typical examples of immunostimulatory oligonucleotides are CpG-motif containing oligonucleotides, single-stranded RNA oligonucleotides, double-stranded RNA oligonucleotides, and double-stranded DNA oligonucleotides. A “CpG-motif” is a cytosine-guanine dinucleotide sequence. In any of the aspects or embodiments of the disclosure, the immunostimulatory oligonucleotide is a toll-like receptor (TLR) agonist (e.g., a toll-like receptor 9 (TLR9) agonist).

As used herein, an “immunosuppressive oligonucleotide” is an oligonucleotide that can suppress (e.g., reduce or inhibit) an immune response. Typical examples of immunosuppressive oligonucleotides are TLR antagonists.

The term “inhibitory oligonucleotide” refers to an oligonucleotide that reduces the production or expression of proteins, such as by interfering with translating mRNA into proteins in a ribosome or that are sufficiently complementary to either a gene or an mRNA encoding one or more of targeted proteins, that specifically bind to (hybridize with) the one or more targeted genes or mRNA thereby reducing expression or biological activity of the target protein. Inhibitory oligonucleotides include, without limitation, isolated or synthetic short hairpin RNA (shRNA or DNA), an antisense oligonucleotide (e.g., antisense RNA or DNA, chimeric antisense DNA or RNA), miRNA and miRNA mimics, small interfering RNA (siRNA), DNA or RNA inhibitors of innate immune receptors, an aptamer, a DNAzyme, or an aptazyme.

An “effective amount” or a “sufficient amount” of a substance is that amount necessary to effect beneficial or desired results, including clinical results, and, as such, an “effective amount” depends upon the context in which it is being applied. In the context of administering an oligonucleotide dendron of the disclosure, for example, an effective amount contains sufficient antigen to elicit an immune response. In some embodiments, an effective amount of an oligonucleotide dendron is an amount sufficient to inhibit gene expression. An effective amount can be administered in one or more doses as described further herein. Efficacy can be shown in an experimental or clinical trial, for example, by comparing results achieved with a substance of interest compared to an experimental control.

The term “dose” as used herein in reference to an antigenic composition refers to a measured portion of the antigenic composition taken by (administered to or received by) a subject at any one time.

The term “vaccination” as used herein refers to the introduction of vaccine into a body of an organism.

An “antigenic composition” is a composition of matter suitable for administration to a human or animal subject (e.g., in an experimental or clinical setting) that is capable of eliciting a specific immune response, e.g., against an antigen, such as a cancer related antigen or a viral antigen. As such, an antigenic composition includes one or more antigens (for example, cancer-related antigens and/or viral antigens) or antigenic epitopes. An antigenic composition can also include one or more additional components capable of eliciting or enhancing an immune response, such as an excipient, carrier, and/or adjuvant. In certain instances, antigenic compositions are administered to elicit an immune response that protects the subject against symptoms or conditions induced by an antigen. In some cases, symptoms or disease caused by an antigen is prevented (or reduced or ameliorated) by inhibiting expansion of cells associated with, e.g., a tumor. In the context of this disclosure, the term antigenic composition will be understood to encompass compositions that are intended for administration to a subject or population of subjects for the purpose of eliciting a protective or palliative immune response against an antigen (for example, cancer-related antigens and/or viral antigens).

“Adjuvant” refers to a substance which, when added to a composition comprising an antigen, nonspecifically enhances or potentiates an immune response to the antigen in the recipient upon exposure. In any of the aspects or embodiments of the disclosure, the oligonucleotide dendrons provided herein comprise immunostimulatory oligonucleotides (for example and without limitation, a CpG oligonucleotide such as CpG-1826) as adjuvants and comprise one or more antigens (e.g., one or more cancer-related antigens, one or more viral antigens, or a combination thereof). Other common adjuvants that may be used in the compositions of the disclosure include suspensions of minerals (alum, aluminum hydroxide, aluminum phosphate) onto which an antigen is adsorbed; emulsions, including water-in-oil, and oil-in-water (and variants thereof, including double emulsions and reversible emulsions), liposaccharides, lipopolysaccharides, Pattern Recognition Receptor (PRR) agonists (e.g., NALP3. RIG-I-like receptors (RIG-I and MDA5), and various combinations of such components.

An “immune response” is a response of a cell of the immune system, such as a B cell, T cell, or monocyte, to a stimulus, such as an antigen (e.g., formulated as an antigenic composition or a vaccine). An immune response can be a B cell response, which results in the production of specific antibodies, such as antigen specific neutralizing antibodies. An immune response can also be a T cell response, such as a CD4+ response or a CD8+ response. B cell and T cell responses are aspects of a “cellular” immune response. An immune response can also be a “humoral” immune response, which is mediated by antibodies. In some cases, the response is specific for a particular antigen (that is, an “antigen-specific response”). A “protective immune response” is an immune response that inhibits a detrimental function or activity of an antigen, or decreases symptoms (including death) that result from the antigen. A protective immune response can be measured, for example, by immune assays using a serum sample from an immunized subject for testing the ability of serum antibodies for inhibition of tumor cell expansion, such as: ELISA-neutralization assay, antibody dependent cell-mediated cytotoxicity assay (ADCC), complement-dependent cytotoxicity (CDC), antibody dependent cell-mediated phagocytosis (ADCP), enzyme-linked immunospot (ELISpot). In addition, vaccine efficacy can be tested by measuring the T cell response CD4+ and CD8+ after immunization, using flow cytometry (FACS) analysis or ELISpot assay. The protective immune response can be tested by measuring resistance to antigen challenge in vivo in an animal model. In humans, a protective immune response can be demonstrated in a population study, comparing measurements of symptoms, morbidity, mortality, etc. in treated subjects compared to untreated controls. Exposure of a subject to an immunogenic stimulus, such as an antigen (e.g., formulated as an antigenic composition or vaccine), elicits a primary immune response specific for the stimulus, that is, the exposure “primes” the immune response. A subsequent exposure, e.g., by immunization, to the stimulus can increase or “boost” the magnitude (or duration, or both) of the specific immune response. Thus, “boosting” a preexisting immune response by administering an antigenic composition increases the magnitude of an antigen-specific response, (e.g., by increasing antibody titer and/or affinity, by increasing the frequency of antigen specific B or T cells, by inducing maturation effector function, or a combination thereof).

### Oligonucleotide Dendrons

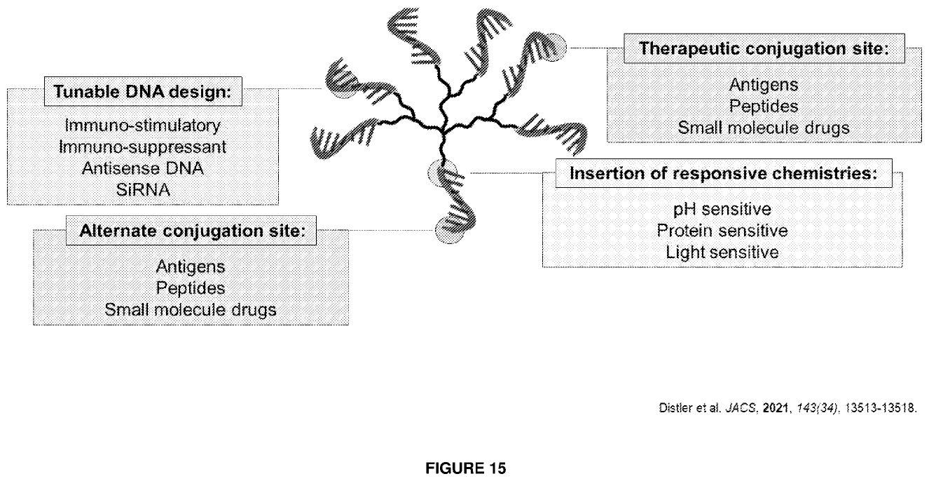

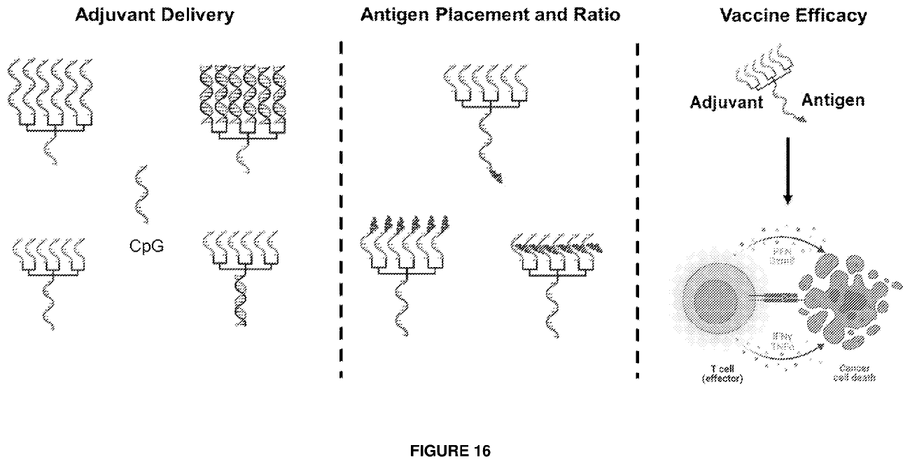

The present disclosure provides oligonucleotide dendrons as molecularly precise therapeutics (e.g., vaccines). The oligonucleotide dendrons disclosed herein are versatile, in that they may in various embodiments comprise one or more conjugation sites to which additional agents (e.g., antigens, peptides) are attached. The oligonucleotide dendrons of the disclosure also comprise a tunable oligonucleotide (e.g., DNA) design. See, for example, FIG. 15. As depicted in FIG. 16, the design parameters of the oligonucleotide dendron can be systematically studied to yield potent molecular vaccines (e.g., cancer vaccines). Parameters such as, but not limited to, the design of adjuvant delivery and antigen placement and ratio factor into ultimate vaccine efficacy. In any of the aspects or embodiments of the disclosure, a molecular nanostructure is provided comprising an adjuvant oligonucleotide (e.g., DNA) strand that splits into multiple oligonucleotide (e.g., DNA) branches with varied number of conjugated additional agents (e.g., antigens, peptides). In any of the aspects or embodiments of the disclosure, an oligonucleotide dendron is not attached to a nanoparticle.

In any of the aspects or embodiments herein, the present disclosure provides oligonucleotide dendrons. In some aspects, the disclosure provides an oligonucleotide dendron comprising an oligonucleotide stem linked to a plurality of oligonucleotide branches through one or more of a doubler moiety, a trebler moiety, or a combination thereof. In some aspects, the disclosure provides an oligonucleotide dendron comprising an oligonucleotide stem linked to a plurality of oligonucleotide branches through one or more of a doubler moiety, a trebler moiety, or a combination thereof, wherein one or more of the plurality of oligonucleotide branches comprises an additional agent, and wherein the additional agent is conjugated at an internal location that is anywhere along the length of the one or more of the plurality of oligonucleotide branches that is not an end of the one or more of the plurality of oligonucleotide branches. In further aspects, the disclosure provides an oligonucleotide dendron comprising an oligonucleotide stem linked to a plurality of oligonucleotide branches through one or more of a doubler moiety, a trebler moiety, or a combination thereof, wherein a free end of the one or more of the plurality of oligonucleotide branches is conjugated to an additional agent. In some aspects, the disclosure provides an oligonucleotide dendron comprising an oligonucleotide stem linked to a plurality of oligonucleotide branches through one or more of a doubler moiety, a trebler moiety, or a combination thereof, wherein one or more of the plurality of oligonucleotide branches comprises an additional agent, and wherein (A) the additional agent is conjugated at an internal location that is anywhere along the length of the one or more of the plurality of oligonucleotide branches that is not an end of the one or more of the plurality of oligonucleotide branches, (B) a free end of the one or more of the plurality of oligonucleotide branches is conjugated to the additional agent, or a combination thereof. In some embodiments, the additional agent is conjugated at an internal location that is anywhere along the length of the one or more of the plurality of oligonucleotide branches that is not an end of the one or more of the plurality of oligonucleotide branches. In further embodiments, a free end of the one or more of the plurality of oligonucleotide branches is conjugated to the additional agent. In some embodiments, all of the plurality of oligonucleotide branches comprises an additional agent. In various embodiments, the oligonucleotide dendron is a DNA dendron, a RNA dendron, a modified oligonucleotide dendron, or a combination thereof. In general, and by way of example, oligonucleotide dendrons of the disclosure may be synthesized using an automated oligonucleotide synthesizer on controlled pore glass (CPG) beads, commonly used in solid-phase oligonucleotide synthesis. Oligonucleotide (e.g., DNA) synthesis involves a series of coupling steps performed on each nucleotide base added to the structure. This comprises (1) a coupling step which attaches a new base to the previous one, (2) a capping step which deactivates any unreacted material, (3) an oxidation step which forms the characteristic phosphate backbone of DNA, and (4) a detritylation step which prepares the newly added base for the next addition. To produce oligonucleotide dendrons, branching units (e.g., doubler moieties, trebler moieties, or a combination thereof), are added into this sequence of nucleotide bases at the desired location. As a result, a stem region is initially synthesized as desired, then the branching units are used to create a plurality of oligonucleotides, and finally the branches are synthesized following the same oligonucleotide (e.g., DNA) synthesis cycle. By changing the type and number of branching units used, an oligonucleotide dendron, with any number of branches, can be synthesized. In various embodiments, conjugation sites are added to the 3′ end (stem) or the 5′ end (branches) of the oligonucleotide dendron such that the oligonucleotide stem and/or the oligonucleotide branches may be conjugated to an additional agent, as described herein. In various embodiments, an additional agent is conjugated either directly to the conjugation site or through a cross linker that connects the conjugation site on the dendron to the conjugation site on the agent. In some embodiments, an oligonucleotide stem is conjugated to the oligonucleotide dendron as an additional agent. In further embodiments, one or more oligonucleotide branches are conjugated to the oligonucleotide dendron as an additional agent(s). In further embodiments, an oligonucleotide stem and one or more oligonucleotide branches are conjugated to the oligonucleotide dendron as additional agents. An oligonucleotide dendron of the disclosure comprises or consists of, in various embodiments, about, at least about, or less than about 1, 2, 3, 4, 5, 6, 7, 8, 9, 10, 11, 12, 13, 14, 15, 16, 17, 18, 19, 20, 21, 22, 23, 24, 25, 26, 27 or more additional agents (e.g., antigens). Alternatively, the number of additional agents (e.g., antigens) associated with an oligonucleotide dendron of the disclosure is expressed as a ratio of adjuvant:antigen. Accordingly, it is contemplated that in various embodiments an oligonucleotide dendron of the disclosure comprises an adjuvant: antigen ratio that is about 30:1, 27:1, 20:1, 15:1, 10:1, 9:1, 8:1, 6:1, 2:1, 1:1, 1:2, 1:6, 1:8, 1:9, 1:10, 1:15, 1:20, 1:27, or 1:30. In further embodiments, the adjuvant: antigen ratio is about 10:1 to about 1:10, or about 6:1 to about 1:6, or about 5:1 to about 1:5, or about 20:1 to about 1:20, or about 8:1 to about 1:8.

Additional description of oligonucleotide dendron synthesis is provided in Example 1, below. Examples of doubler moieties that may be used in the synthesis of an oligonucleotide dendron are shown below and are available from Glen Research, Sterling, VA. Note that the structures below represent the doubler moiety prior to incorporation into the oligonucleotide dendron.

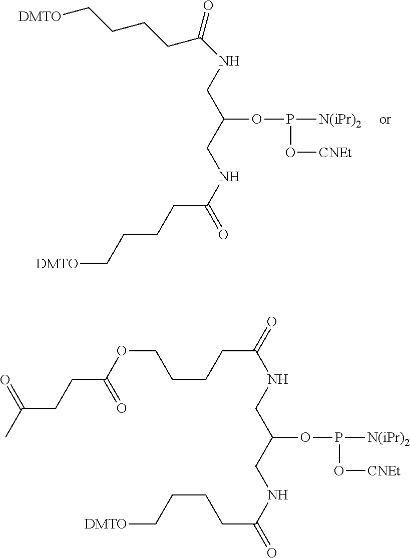

DMT=4,4′-dimethoxytriryl; O=oxygen; C=carbon; N=nitrogen; Et=ethyl; iPr=isopropyl

More generally, it is contemplated that in any of the aspects or embodiments of the disclosure a doubler moiety comprises the following structure:

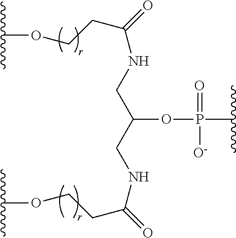

where each r can be 0, 1, 2, 3, 4, 5 or 6. P can be conjugated to an oligonucleotide portion of a dendron of the disclosure (e.g., oligonucleotide stem and/or oligonucleotide branch) or to an oxygen end group of another doubler moiety or trebler moiety and each oxygen end group of the oligonucleotide branches can be connected to a dendron or P of a further doubler or trebler. In some embodiments, each r=3.

Examples of trebler moieties that may be used in the synthesis of an oligonucleotide dendron are shown below and are available from Glen Research, Sterling, VA. Note that the structures below represent the trebler moiety prior to incorporation into the oligonucleotide dendron.

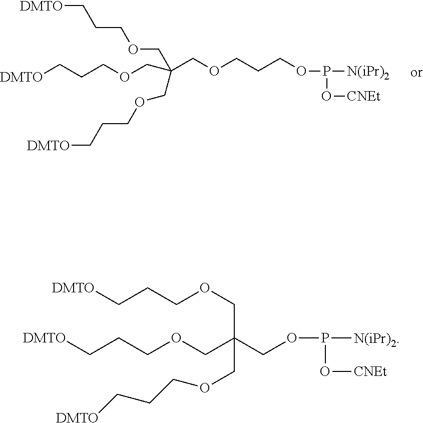

DMT=4,4′-dimethoxytriryl; O=oxygen; C=carbon; N=nitrogen; Et=ethyl; iPr=isopropyl

More generally, it is contemplated that in any of the aspects or embodiments of the disclosure a trebler moiety comprises the following structure:

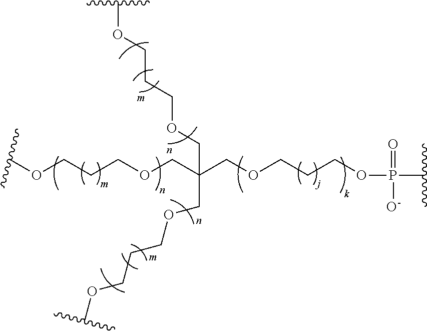

where each m can be 0, 1, 2, or 3; each n can be 1, 2, 3, or 4; j can be 0, 1, 2, or 3; and k can be 1, 2, 3, or 4. In some embodiments, j=1, k=1, each m=1 and each n=1.

In any of the aspects or embodiments of the disclosure, an oligonucleotide dendron comprises one or more additional agents (e.g., one or more antigens) as described herein. In some embodiments, the additional agent is conjugated to an end (5′ end or 3′ end) of the oligonucleotide stem and/or one or more or all of the oligonucleotide branches of the oligonucleotide dendron. In further embodiments, the additional agent is conjugated to a midpoint of the oligonucleotide stem and/or one or more or all of the oligonucleotide branches of the oligonucleotide. Combinations of the foregoing are also contemplated. By “midpoint” is meant that the additional agent is conjugated at an internal location that is anywhere along the length of an oligonucleotide (e.g., oligonucleotide stem, oligonucleotide branch) of the dendron that is not an end (terminus) of the oligonucleotide.

The antigen, in various embodiments, is a cancer related antigen, a viral antigen, a bacterial antigen, or a combination thereof. In some embodiments, the viral antigen is a coronavirus antigen, an influenza virus, a herpes virus (e.g., herpes zoster), a human papilloma virus (HPV), a human immunodeficiency virus (HIV), measles, mumps, and Rubella (MMR), a variant of any of the foregoing, or a combination thereof. In further embodiments, the coronavirus is SARS-COV-2 or a variant thereof. In related embodiments, the antigen is derived from a SARS-COV-2 spike receptor binding domain or any variant thereof. In still further embodiments, the viral antigen is or is derived from SARS-COV-2 envelope protein, SARS-COV-2 nucleocapsid protein, SARS-COV-2 membrane protein, a variant or fragment of any of the foregoing, or a combination thereof. As used herein, a “variant” refers to a genetic variant that comprises one or more mutations relative to a wild type amino acid sequence. Thus, in any of the aspects or embodiments of the disclosure, a viral antigen comprises or consists of a nucleotide or an amino acid sequence that is at least 80%, at least 85%, at least 90%, at least 95%, at least 99%, or 100% identical to a reference or wild type sequence.

In various embodiments, the cancer related antigen is a melanoma related antigen, a HPV related antigen, a colon cancer antigen, a lymphoma antigen, a prostate cancer related antigen (e.g., prostate-specific membrane antigen), a glioblastoma antigen, an ovarian cancer related antigen, a breast cancer related antigen, a hepatocellular carcinoma related antigen, a lung cancer related antigen, a bowel cancer related antigen, or human papillomavirus (HPV) E6 or E7 nuclear protein.

In various embodiments, the antigen is conjugated to the oligonucleotide stem of the oligonucleotide dendron. In some embodiments, the antigen is conjugated to one or more or all of the oligonucleotide branches of the oligonucleotide dendron. In some embodiments, the antigen is conjugated to one or more or all of the oligonucleotide branches and the oligonucleotide stem of the oligonucleotide dendron.

### Compositions

The disclosure also provides compositions that comprise an oligonucleotide dendron of the disclosure, or a plurality thereof. In some embodiments, the composition further comprises a pharmaceutically acceptable carrier. The term “carrier” refers to a vehicle within which the oligonucleotide dendron as described herein is administered to a subject. Any conventional media or agent that is compatible with the oligonucleotide dendrons according to the disclosure can be used. The term carrier encompasses diluents, excipients, adjuvants and a combination thereof.

### Additional Agents

The oligonucleotide dendrons provided herein optionally include an additional agent. The additional agent is, in various embodiments, simply associated with the oligonucleotide stem of an oligonucleotide dendron and/or one or more or all of the oligonucleotide branches of an oligonucleotide dendron. In some embodiments, the additional agent is associated with the end of an oligonucleotide branch that is not connected to an oligonucleotide stem. In some embodiments, the additional agent is covalently associated with the oligonucleotide stem. In some embodiments, the additional agent is non-covalently associated with the oligonucleotide stem. It is contemplated that this additional agent is in one aspect covalently associated with the one or more or all of the plurality of oligonucleotide branches, or in the alternative, non-covalently associated with the one or more of the plurality of oligonucleotide branches. However, it is understood that the disclosure provides oligonucleotide dendrons wherein one or more additional agents are both covalently and non-covalently associated with the oligonucleotide stem and/or the one or more or all of the plurality of oligonucleotide branches. It will also be understood that non-covalent associations include hybridization, protein binding, and/or hydrophobic interactions.

Functional groups, such as primary amines, thiols, NHS esters, and DBCO, can be placed on the oligonucleotide dendron structure for facile functionalization with other therapeutic agents. For example and without limitation, peptides can be conjugated to the dendron by reacting the chemical functional group with a cystine or lysine on the peptide. Conjugates may be purified by PAGE and characterized by PAGE and HPLC.

Additional agents contemplated by the disclosure include without limitation a protein (e.g., an antigen, a therapeutic protein), a small molecule, a peptide, an oligonucleotide (e.g., an immunostimulatory oligonucleotide, an inhibitory oligonucleotide), or a combination thereof. In some embodiments, an additional agent is an oligonucleotide stem and/or an oligonucleotide branch. These additional agents are described herein.

The term “small molecule,” as used herein, refers to a chemical compound or a drug, or any other low molecular weight organic compound, either natural or synthetic. By “low molecular weight” is meant compounds having a molecular weight of less than 1500 Daltons, typically between 100 and 700 Daltons.

### Oligonucleotides

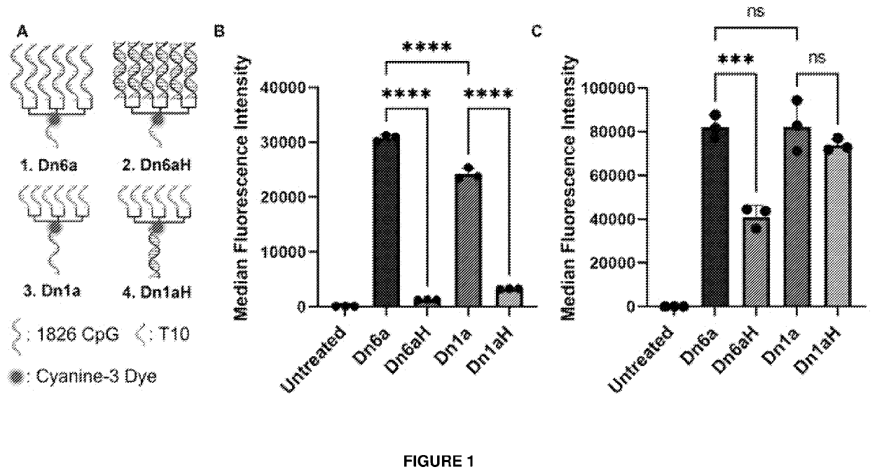

Oligonucleotide dendrons of the disclosure are nucleic acid structures comprising an oligonucleotide stem to which a plurality of oligonucleotide branches is linked via one or more doubler moieties, trebler moieties, or a combination thereof. See, e.g., FIG. 1A. Thus, an oligonucleotide dendron of the disclosure is a single oligonucleotide molecule having a dendritic architecture. In various embodiments, an oligonucleotide dendron further comprises an additional agent (e.g., an antigen). It will be understood that all features of oligonucleotides described herein (e.g., type (DNA/RNA), single/double stranded, length, sequence, modified forms) apply to all oligonucleotides described herein, including oligonucleotide dendrons, oligonucleotide stems, and oligonucleotide branches. In some embodiments, the oligonucleotide stem and/or one or more or all of the oligonucleotide branches of an oligonucleotide dendron comprises a (GGX), nucleotide sequence, wherein n is 2-20 and X is a nucleobase (A, C, T, G, or U). In some embodiments, the (GGX)ₙ nucleotide sequence is on the 5′ end of the oligonucleotide stem and/or one or more or all of the oligonucleotide branches of an oligonucleotide dendron. In some embodiments, the (GGX), nucleotide sequence is on the 3′ end of the oligonucleotide stem and/or one or more or all of the oligonucleotide branches of an oligonucleotide dendron. In some embodiments, the oligonucleotide stem and/or one or more or all of the oligonucleotide branches of an oligonucleotide dendron comprises a (GGT)ₙ nucleotide sequence, wherein n is 2-20. In some embodiments, the (GGT)ₙ nucleotide sequence is on the 5′ end of the oligonucleotide stem and/or one or more or all of the oligonucleotide branches of an oligonucleotide dendron. In some embodiments, the (GGT)ₙ nucleotide sequence is on the 3′ end of the oligonucleotide stem and/or one or more or all of the oligonucleotide branches of an oligonucleotide dendron. In further embodiments, the oligonucleotide stem of an oligonucleotide dendron comprises or consists of a homopolymeric nucleotide sequence. In further embodiments, one or more or all of the oligonucleotide branches of an oligonucleotide dendron comprises or consists of a homopolymeric nucleotide sequence. In some aspects, the homopolymeric sequence comprises a sequence of thymidine residues (polyT), adenine residues (polyA), guanine residues (polyG), cytosine residues (polyC), or uridine residues (polyU). In further aspects, the homopolymeric sequence comprises or consists of two nucleotide residues. In various aspects, the homopolymeric sequence comprises or consists of about, at least about, or less than about 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotide residues. In some embodiments, the homopolymeric sequence is about 3 to about 20, or about 3 to about 15, or about 3 to about 10, or about 5 to about 20, or about 5 to about 15, or about 5 to about 10 nucleotide residues. In some embodiments, the oligonucleotide stem of an oligonucleotide dendron is an immunostimulatory oligonucleotide (e.g., an oligonucleotide comprising a CpG nucleotide sequence) and one or more or all of the oligonucleotide branches of the oligonucleotide dendron comprises a homopolymeric (e.g., polyT) nucleotide sequence. In further embodiments, the oligonucleotide stem and/or one or more or all of the oligonucleotide branches comprises an additional agent (e.g., an antigen) conjugated thereto. In some embodiments, the oligonucleotide stem and each oligonucleotide branch of an oligonucleotide dendron comprises a functional nucleotide sequence. A “functional” nucleotide sequence is a sequence that provides a biological function such as immune regulation or gene regulation.

In various embodiments, an oligonucleotide dendron comprises about 2 to about 27 oligonucleotide branches. In further embodiments, an oligonucleotide dendron comprises about 2 to about 25, or about 2 to about 23, or about 2 to about 20, or about 2 to about 18, or about 2 to about 16, or about 2 to about 15, or about 2 to about 13, or about 2 to about 10, or about 2 to about 8, or about 2 to about 7, or about 2 to about 5, or about 2 to about 4, or about 2 to about 3 oligonucleotide branches. In further embodiments, an oligonucleotide dendron comprises at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, or at least 27 oligonucleotide branches. In further embodiments, an oligonucleotide dendron comprises less than 27, less than 26, less than 25, less than 24, less than 23, less than 22, less than 21, less than 20, less than 19, less than 18, less than 17, less than 16, less than 15, less than 14, less than 13, less than 12, less than 11, less than 10, less than 9, less than 8, less than 7, less than 6, less than 5, less than 4, or less than 3 oligonucleotide branches. In some embodiments, an oligonucleotide dendron comprises or consists of 2, 3, 4, 5, 6, 7, 8, 9, 10, 11, 12, 13, 14, 15, 16, 17, 18, 19, 20, 21, 22, 23, 24, 25, 26, or 27 oligonucleotide branches. In further embodiments, an oligonucleotide dendron comprises 2, 3, 4, 6, 8, 9, 12, 18, or 27 oligonucleotide branches. In still further embodiments, an oligonucleotide dendron consists of 2, 3, 4, 6, 8, 9, 12, 18, or 27 oligonucleotide branches. In some embodiments, an oligonucleotide dendron consists of 6 branches. In some embodiments, an oligonucleotide dendron consists of 9 branches.

Oligonucleotides (e.g., an oligonucleotide dendron, an oligonucleotide stem, or an oligonucleotide branch) contemplated for use according to the disclosure include, in various embodiments, DNA oligonucleotides, RNA oligonucleotides, modified forms thereof, or a combination thereof. Thus, in some embodiments, the oligonucleotide dendron is a DNA dendron, a RNA dendron, a modified oligonucleotide dendron, or a combination thereof. In some embodiments, the oligonucleotide stem is RNA and each oligonucleotide branch that is attached to the oligonucleotide stem through a doubler moiety, a trebler moiety, or a combination thereof is DNA. In some embodiments, the oligonucleotide stem is DNA and each oligonucleotide branch that is attached to the oligonucleotide stem through a doubler moiety, a trebler moiety, or a combination thereof is RNA. Thus, in any of the aspects or embodiments of the disclosure, the oligonucleotide stem portion of an oligonucleotide dendron may be a different nucleic acid class than the oligonucleotide branches that are attached to the oligonucleotide stem through a doubler moiety, a trebler moiety, or a combination thereof, but each oligonucleotide branch in the oligonucleotide dendron is the same nucleic acid class (e.g., the oligonucleotide stem can be DNA while each oligonucleotide branch is RNA).

In any aspects or embodiments described herein, an oligonucleotide is single-stranded, double-stranded, or partially double-stranded. Thus, in various embodiments, oligonucleotide stems and oligonucleotide branches can be single, double, or partially double stranded. In some embodiments, one or more or all of the oligonucleotide branches of an oligonucleotide dendron is single stranded and the oligonucleotide stem of the oligonucleotide dendron is also single stranded. In some embodiments, one or more or all of the oligonucleotide branches of an oligonucleotide dendron is double stranded while the oligonucleotide stem of the oligonucleotide dendron is single stranded. In some embodiments, one or more or all of the oligonucleotide branches of an oligonucleotide dendron is double stranded while the oligonucleotide stem of the oligonucleotide dendron is double stranded. In some embodiments, one or more or all of the oligonucleotide branches of an oligonucleotide dendron is single stranded while the oligonucleotide stem of the oligonucleotide dendron is double stranded. Modified forms of oligonucleotides are also contemplated which include those having at least one modified internucleotide linkage. In some embodiments, the oligonucleotide is all or in part a peptide nucleic acid. Other modified internucleoside linkages include at least one phosphorothioate linkage. Still other modified oligonucleotides include those comprising one or more universal bases. “Universal base” refers to molecules capable of substituting for binding to any one of A, C, G, T and U in nucleic acids by forming hydrogen bonds without significant structure destabilization. The oligonucleotide incorporated with the universal base analogues is able to function, e.g., as a probe in hybridization. Examples of universal bases include but are not limited to 5′-nitroindole-2′-deoxyriboside, 3-nitropyrrole, inosine and hypoxanthine.

The term “nucleotide” or its plural as used herein is interchangeable with modified forms as discussed herein and otherwise known in the art. The term “nucleobase” or its plural as used herein is interchangeable with modified forms as discussed herein and otherwise known in the art. Nucleotides or nucleobases comprise the naturally occurring nucleobases A, G, C, T, and U. Non-naturally occurring nucleobases include, for example and without limitations, xanthine, diaminopurine, 8-oxo-N6-methyladenine, 7-deazaxanthine, 7-deazaguanine, N4,N4-ethanocytosin, N′,N′-ethano-2,6-diaminopurine, 5-methylcytosine (mC), 5-(C3-C6)-alkynyl-cytosine, 5-fluorouracil, 5-bromouracil, pseudoisocytosine, 2-hydroxy-5-methyl-4-tr-iazolopyridin, isocytosine, isoguanine, inosine and the “non-naturally occurring” nucleobases described in Benner et al., U.S. Pat. No. 5,432,272 and Susan M. Freier and Karl-Heinz Altmann, 1997, Nucleic Acids Research, vol. 25: pp 4429-4443. The term “nucleobase” also includes not only the known purine and pyrimidine heterocycles, but also heterocyclic analogues and tautomers thereof. Further naturally and non-naturally occurring nucleobases include those disclosed in U.S. Pat. No. 3,687,808 (Merigan, et al.), in Chapter 15 by Sanghvi, in Antisense Research and Application, Ed. S. T. Crooke and B. Lebleu, CRC Press, 1993, in Englisch et al., 1991, Angewandte Chemie, International Edition, 30:613-722 (see especially pages 622 and 623, and in the Concise Encyclopedia of Polymer Science and Engineering, J. I. Kroschwitz Ed., John Wiley & Sons, 1990, pages 858-859, Cook, Anti-Cancer Drug Design 1991, 6, 585-607, each of which are hereby incorporated by reference in their entirety). In various aspects, oligonucleotides also include one or more “nucleosidic bases” or “base units” which are a category of non-naturally-occurring nucleotides that include compounds such as heterocyclic compounds that can serve like nucleobases, including certain “universal bases” that are not nucleosidic bases in the most classical sense but serve as nucleosidic bases. Universal bases include 3-nitropyrrole, optionally substituted indoles (e.g., 5-nitroindole), and optionally substituted hypoxanthine. Other desirable universal bases include, pyrrole, diazole or triazole derivatives, including those universal bases known in the art.

Examples of oligonucleotides include those containing modified backbones or non-natural internucleoside linkages. Oligonucleotides having modified backbones include those that retain a phosphorus atom in the backbone and those that do not have a phosphorus atom in the backbone. Modified oligonucleotides that do not have a phosphorus atom in their internucleoside backbone are considered to be within the meaning of “oligonucleotide”.

Modified oligonucleotide backbones containing a phosphorus atom include, for example, phosphorothioates, chiral phosphorothioates, phosphorodithioates, phosphotriesters, aminoalkylphosphotriesters, methyl and other alkyl phosphonates including 3′-alkylene phosphonates, 5′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, selenophosphates and boranophosphates having normal 3′-5′ linkages, 2′-5′ linked analogs of these, and those having inverted polarity wherein one or more internucleotide linkages is a 3′ to 3′, 5′ to 5′ or 2′ to 2′ linkage. Also contemplated are oligonucleotides having inverted polarity comprising a single 3′ to 3′ linkage at the 3′-most internucleotide linkage, i.e. a single inverted nucleoside residue which may be abasic (the nucleotide is missing or has a hydroxyl group in place thereof). Salts, mixed salts and free acid forms are also contemplated. Representative United States patents that teach the preparation of the above phosphorus-containing linkages include, U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550, 111; 5,563,253; 5,571,799; 5,587,361; 5,194,599; 5,565,555; 5,527,899; 5,721,218; 5,672,697 and 5,625,050, the disclosures of which are incorporated by reference herein.

Modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages; siloxane backbones; sulfide, sulfoxide and sulfone backbones; formacetyl and thioformacetyl backbones; methylene formacetyl and thioformacetyl backbones; riboacetyl backbones; alkene containing backbones; sulfamate backbones; methyleneimino and methylenehydrazino backbones; sulfonate and sulfonamide backbones; amide backbones; and others having mixed N, O, S and CH₂ component parts. See, for example, U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; 5,792,608; 5,646,269 and 5,677,439, the disclosures of which are incorporated herein by reference in their entireties.

In still further embodiments, oligonucleotide mimetics wherein both one or more sugar and/or one or more internucleotide linkage of the nucleotide units are replaced with “non-naturally occurring” groups. The bases of the oligonucleotide are maintained for hybridization. In some aspects, this embodiment contemplates a peptide nucleic acid (PNA). In PNA compounds, the sugar-backbone of an oligonucleotide is replaced with an amide containing backbone. See, for example U.S. Pat. Nos. 5,539,082; 5,714,331; and 5,719,262, and Nielsen et al., Science, 1991, 254, 1497-1500, the disclosures of which are herein incorporated by reference.

In still further embodiments, oligonucleotides are provided with phosphorothioate backbones and oligonucleosides with heteroatom backbones, and including —CH₂—NH—O—CH₂—, —CH₂—N(CH₃)—O—CH₂—, —CH₂—O—N(CH₃)—CH₂—, —CH₂—N(CH₃)—N(CH₃)—CH₂— and —O—N(CH₃)—CH₂—CH₂— described in U.S. Pat. Nos. 5,489,677, and 5,602,240. Also contemplated are oligonucleotides with morpholino backbone structures described in U.S. Pat. No. 5,034,506.

In various forms, the linkage between two successive monomers in the oligonucleotide consists of 2 to 4, desirably 3, groups/atoms selected from-CH₂—, —O—, —S—, —NR^{H}—, >C═O, >C═NR^{H}, >C═S, —Si(R″)₂—, —SO—, —S(O)₂—, —P(O)₂—, —PO(BH₃)—, —P(O,S)—, —P(S)₂—, —PO(R″)—, —PO(OCH₃)—, and —PO(NHR^{H})—, where R^{H} is selected from hydrogen and C₁₋₄-alkyl, and R″ is selected from C₁₋₆-alkyl and phenyl. Illustrative examples of such linkages are —CH₂—CH₂—CH₂—, —CH₂—CO—CH₂—, —CH₂—CHOH—CH₂—, —O—CH₂—O—, —O—CH₂—CH₂—, —O—CH₂—CH═ (including R⁵ when used as a linkage to a succeeding monomer), —CH₂—CH₂—O—, —NR^{H}—CH₂—CH₂, —CH₂—CH₂—NR^{H}—, —CH₂—NR^{H}—CH₂-—, —O—CH₂—CH₂—NR^{H}—, —NR^{H}—CO—O—, —NR^{H}—CO—NR^{H}—, —NR^{H}—CS—NR^{H}—, —NR^{H}—C(═NR^{H})—NR^{H}—, —NR^{H}—CO—CH₂—NR^{H}—O—CO—O—, —O—CO—CH₂—O—, —O—CH₂—CO—O—, —CH₂—CO—NR^{H}—, —O—CO—NR^{H}—, —NR^{H}—CO—CH₂—, —O—CH₂—CO—NR^{H}—, —O—CH₂—CH₂—NR^{H}—, —CH═N—O—, —CH₂—NR^{H}—O—, —CH₂—O—N═ (including R⁵ when used as a linkage to a succeeding monomer), —CH₂—O—NR^{H}—, —CO—NR^{H}—CH₂—, —CH₂—NR^{H}—O—, —CH₂—NR^{H}—CO—, —O—NR^{H}—CH₂—, —O—NR^{H}, —O—CH₂—S—, —S—CH₂—O—, —CH₂—CH₂—S—, —O—CH₂—CH₂—S—, —S—CH₂—CH═ (including R⁵ when used as a linkage to a succeeding monomer), —S—CH₂—CH₂—, —S—CH₂—CH₂—, —O—, —S—CH₂—CH₂—S—, —CH₂—S—CH₂—, —CH₂—SO—CH₂—, —CH₂—SO₂—CH₂—, —O—SO—O—, —O—S(O)₂—O—, —O—S(O)₂—CH₂—, —O—S(O)₂—NR^{H}—, —NR^{H}—S(O)₂—CH₂—; —O—S(O)₂—CH₂—, —O—P(O)₂—O—, —O—P(O,S)—O—, —O—P(S)₂—O—, —S—P(O)₂—O—, —S—P(O,S)—O—, —S—P(S)₂—O—, —O—P(O)₂—S—, —O—P(O,S)—S—, —O—P(S)₂—S—, —S—P(O)₂—S—, —S—P(O,S)—S—, —S—P(S)₂—S—, —O—PO(R″)—O—, —O—PO(OCH₃)—O—, —O—PO(O CH₂CH₃)—O—, —O—PO(OCH₂CH₂S—R)—O—, —O—PO(BH₃)—O—, —O—PO(NHR^{N})—O—, —O—P(O)₂—NR^{H} H—, —NR^{H}—P(O)₂—O—, —O—P(O,NR^{H})—O—, —

CH₂—P(O)₂—O—, —O—P(O)₂—CH₂—, and —O—Si(R″)₂—O—; among which —CH₂—CO—NR^{H}—, —CH₂—NR^{H}—O—, —S—CH₂—O—, —O—P(O)₂—O—O—P(—O,S)—O—, —O—P(S)₂—O—, —NR^{H} P(O)₂—O—, —O—P(O,NR^{H})—O—, —O—PO(R″)—O—, —O—PO(CH₃)—O—, and —O—PO(NHR^{N})—O—, where R^{H} is selected form hydrogen and C₁₋₄-alkyl, and R″ is selected from C₁₋₆-alkyl and phenyl, are contemplated. Further illustrative examples are given in Mesmaeker et. al., Current Opinion in Structural Biology 1995, 5, 343-355 and Susan M. Freier and Karl-Heinz Altmann, Nucleic Acids Research, 1997, vol 25, pp 4429-4443.

Still other modified forms of oligonucleotides are described in detail in U.S. patent application No. 20040219565, the disclosure of which is incorporated by reference herein in its entirety.

Modified oligonucleotides may also contain one or more substituted sugar moieties. In certain aspects, oligonucleotides comprise one of the following at the 2′ position: OH; F; O-, S-, or N-alkyl; O-, S-, or N-alkenyl; O-, S- or N-alkynyl; or O-alkyl-O-alkyl, wherein the alkyl, alkenyl and alkynyl may be substituted or unsubstituted C₁ to C₁₀ alkyl or C₂ to C₁₀ alkenyl and alkynyl. Other embodiments include O[(CH₂)ₙO]ₘCH₃, O(CH₂)ₙOCH₃, O(CH₂)ₙNH₂, O(CH₂)ₙCH₃, O(CH₂)ₙONH₂, and O(CH₂)ₙON[(CH₂)ₙCH₃]₂, where n and m are from 1 to about 10. Other oligonucleotides comprise one of the following at the 2′ position: C1 to C10 lower alkyl, substituted lower alkyl, alkenyl, alkynyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH2, heterocycloalkyl, heterocycloalkaryl, aminoalkylamino, polyalkylamino, substituted silyl, or an RNA cleaving group. In one aspect, a modification includes 2′-methoxyethoxy (2′-O-CH₂CH₂OCH₃, also known as 2′-O-(2-methoxyethyl) or 2′-MOE) (Martin et al., Helv. Chim. Acta, 1995, 78, 486-504) i.e., an alkoxyalkoxy group. Other modifications include 2′-dimethylaminooxyethoxy, i.e., a O(CH₂)₂ON(CH₃)₂ group, also known as 2′-DMAOE, and 2′-dimethylaminoethoxyethoxy (also known in the art as 2′-O-dimethyl-amino-ethoxy-ethyl or 2′-DMAEOE), i.e., 2′-O—CH₂—O—CH₂—N(CH₃)₂.

Still other modifications include 2′-methoxy (2′-O—CH₃), 2′-aminopropoxy (2′-OCH₂CH₂CH₂NH₂), 2′-allyl(2′-CH₂—CH═CH₂), 2′-O-allyl(2′-O—CH₂—CH═CH₂) and 2′-fluoro (2′-F). The 2′-modification may be in the arabino (up) position or ribo (down) position. In one aspect, a 2′-arabino modification is 2′-F. Similar modifications may also be made at other positions on the oligonucleotide, for example, at the 3′ position of the sugar on the 3′ terminal nucleotide or in 2′-5′ linked oligonucleotides and the 5′ position of 5′ terminal nucleotide. Oligonucleotides may also have sugar mimetics such as cyclobutyl moieties in place of the pentofuranosyl sugar. See, for example, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; 5,792,747; and 5,700,920, the disclosures of which are incorporated by reference in their entireties herein.

In some aspects, a modification of the sugar includes Locked Nucleic Acids (LNAs) in which the 2′-hydroxyl group is linked to the 3′ or 4′ carbon atom of the sugar ring, thereby forming a bicyclic sugar moiety. The linkage is in certain aspects is a methylene (—CH₂—)n group bridging the 2′ oxygen atom and the 4′ carbon atom wherein n is 1 or 2. LNAs and preparation thereof are described in WO 98/39352 and WO 99/14226.

Modified nucleotides are described in EP 1 072 679 and WO 97/12896, the disclosures of which are incorporated herein by reference. Modified nucleobases include without limitation, 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine and 3-deazaguanine and 3-deazaadenine. Further modified bases include tricyclic pyrimidines such as phenoxazine cytidine (1H-pyrimido[5,4-b][1,4]benzoxazin-2 (3H)-one), phenothiazine cytidine (1H-pyrimido[5,4-b][1,4]benzothiazin-2 (3H)-one), G-clamps such as a substituted phenoxazine cytidine (e.g. 9-(2-aminoethoxy)-H-pyrimido[5,4-b][1,4]benzox-azin-2 (3H)-one), carbazole cytidine (2H-pyrimido[4,5-b]indol-2-one), pyridoindole cytidine (H-pyrido[3′,2′:4,5]pyrrolo[2,3-d]pyrimidin-2-one). Modified bases may also include those in which the purine or pyrimidine base is replaced with other heterocycles, for example 7-deaza-adenine, 7-deazaguanosine, 2-aminopyridine and 2-pyridone. Additional nucleobases include those disclosed in U.S. Pat. No. 3,687,808, those disclosed in The Concise Encyclopedia Of Polymer Science And Engineering, pages 858-859, Kroschwitz, J. I., ed. John Wiley & Sons, 1990, those disclosed by Englisch et al., 1991, Angewandte Chemie, International Edition, 30:613, and those disclosed by Sanghvi, Y. S., Chapter 15, Antisense Research and Applications, pages 289-302, Crooke, S. T. and Lebleu, B., ed., CRC Press, 1993. Certain of these bases are useful for increasing binding affinity and include 5-substituted pyrimidines, 6-azapyrimidines and N-2, N-6 and O-6 substituted purines, including 2-aminopropyladenine, 5-propynyluracil and 5-propynylcytosine. 5-methylcytosine substitutions have been shown to increase nucleic acid duplex stability by 0.6-1.2° C. and are, in certain aspects combined with 2′-O-methoxyethyl sugar modifications. See, U.S. Pat. Nos. 3,687,808, 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,594, 121, 5,596,091; 5,614,617; 5,645,985; 5,830,653; 5,763,588; 6,005,096; 5,750,692 and 5,681,941, the disclosures of which are incorporated herein by reference.

Methods of making polynucleotides of a predetermined sequence are well-known. See, e.g., Sambrook et al., Molecular Cloning: A Laboratory Manual (2nd ed. 1989) and F. Eckstein (ed.) Oligonucleotides and Analogues, 1st Ed. (Oxford University Press, New York, 1991). Solid-phase synthesis methods are preferred for both polyribonucleotides and polydeoxyribonucleotides (the well-known methods of synthesizing DNA are also useful for synthesizing RNA). Polyribonucleotides can also be prepared enzymatically. Non-naturally occurring nucleobases can be incorporated into the polynucleotide, as well. See, e.g., U.S. Pat. No. 7,223,833; Katz, J. Am. Chem. Soc., 74:2238 (1951); Yamane, et al., J. Am. Chem. Soc., 83:2599 (1961); Kosturko, et al., Biochemistry, 13:3949 (1974); Thomas, J. Am. Chem. Soc., 76:6032 (1954); Zhang, et al., J. Am. Chem. Soc., 127:74-75 (2005); and Zimmermann, et al., J. Am. Chem. Soc., 124:13684-13685 (2002).

In various aspects, an oligonucleotide of the disclosure (e.g., an oligonucleotide stem, an oligonucleotide branch), or a modified form thereof, is generally about 10 nucleotides to about 100 nucleotides in length. More specifically, an oligonucleotide of the disclosure is about 10 to about 90 nucleotides in length, about 10 to about 80 nucleotides in length, about 10 to about 70 nucleotides in length, about 10 to about 60 nucleotides in length, about 10 to about 50 nucleotides in length about 10 to about 45 nucleotides in length, about 10 to about 40 nucleotides in length, about 10 to about 35 nucleotides in length, about 10 to about 30 nucleotides in length, about 10 to about 25 nucleotides in length, about 10 to about 20 nucleotides in length, about 10 to about 15 nucleotides in length, and all oligonucleotides intermediate in length of the sizes specifically disclosed to the extent that the oligonucleotide is able to achieve the desired result. In further embodiments, an oligonucleotide of the disclosure is about 5 nucleotides to about 1000 nucleotides in length. In further embodiments, an oligonucleotide of the disclosure is about 5 to about 900 nucleotides in length, about 5 to about 800 nucleotides in length, about 5 to about 700 nucleotides in length, about 5 to about 600 nucleotides in length, about 5 to about 500 nucleotides in length about 5 to about 450 nucleotides in length, about 5 to about 400 nucleotides in length, about 5 to about 350 nucleotides in length, about 5 to about 300 nucleotides in length, about 5 to about 250 nucleotides in length, about 5 to about 200 nucleotides in length, about 5 to about 150 nucleotides in length, about 5 to about 100 nucleotides in length, about 5 to about 90 nucleotides in length, about 5 to about 80 nucleotides in length, about 5 to about 70 nucleotides in length, about 5 to about 60 nucleotides in length, about 5 to about 50 nucleotides in length, about 5 to about 40 nucleotides in length, about 5 to about 30 nucleotides in length, about 5 to about 20 nucleotides in length, about 5 to about 10 nucleotides in length, and all oligonucleotides intermediate in length of the sizes specifically disclosed to the extent that the oligonucleotide is able to achieve the desired result. Accordingly, in various embodiments, an oligonucleotide of the disclosure is or is at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 200, 300, 400, 500, 600, 700, 800, 900, 1000, or more nucleotides in length. In further embodiments, an oligonucleotide of the disclosure is less than 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 200, 300, 400, 500, 600, 700, 800, 900, 1000, or more nucleotides in length. In further embodiments, an oligonucleotide stem of the disclosure is about 1-50 nucleotides, about 1-40 nucleotides, about 1-30 nucleotides, about 1-20 nucleotides, about 1-10 nucleotides, about 5-50 nucleotides, about 5-40 nucleotides, about 5-35 nucleotides, about 5-30 nucleotides, about 5-25 nucleotides, about 5-20 nucleotides, about 5-10 nucleotides, about 10-15 nucleotides, about 10-20 nucleotides, about 10-25 nucleotides, or about 10-30 nucleotides in length. In some embodiments, an oligonucleotide stem of the disclosure is or is about 15 nucleotides in length. In further embodiments, an oligonucleotide branch of the disclosure is about 1-30 nucleotides, about 1-25, about 1-20 nucleotides, about 1-15 nucleotides, about 1-10 nucleotides, about 1-5 nucleotides, about 5-10 nucleotides, about 5-15 nucleotides, about 5-20 nucleotides, about 5-25 nucleotides, about 5-30 nucleotides, about 10-15 nucleotides, about 10-20 nucleotides, about 10-25 nucleotides, or about 10-30 nucleotides in length. In some embodiments, an oligonucleotide branch of the disclosure is or is about 10 nucleotides in length.

### Spacers.

In some aspects, an oligonucleotide (e.g., an oligonucleotide stem, an oligonucleotide branch) comprises a spacer. In some embodiments, an oligonucleotide branch is attached to a doubler and/or a trebler moiety through a spacer. In some aspects, the spacer when present is an organic moiety. In some aspects, the spacer is a polymer, including but not limited to a water-soluble polymer, a nucleic acid, a polypeptide, an oligosaccharide, a carbohydrate, a lipid, an ethylglycol, or a combination thereof. In any of the aspects or embodiments of the disclosure, the spacer is an oligo (ethylene glycol)-based spacer. In various embodiments, an oligonucleotide comprises 1, 2, 3, 4, 5, or more spacer (e.g., Spacer-18 (hexaethyleneglycol)) moieties. In further embodiments, the spacer is an alkane-based spacer (e.g., C12). In some embodiments, the spacer is an oligonucleotide spacer (e.g., T5). An oligonucleotide spacer may have any sequence that does not interfere with the ability of the oligonucleotide to perform an intended function (e.g., inhibit gene expression). In certain aspects, the bases of the oligonucleotide spacer are all adenylic acids, all thymidylic acids, all cytidylic acids, all guanylic acids, all uridylic acids, or all some other modified base. In various embodiments, the length of the spacer is or is equivalent to at least about 2 nucleotides, at least about 3 nucleotides, at least about 4 nucleotides, at least about 5 nucleotides, 5-10 nucleotides, 10 nucleotides, 20 nucleotides, 10-30 nucleotides, or even greater than 30 nucleotides.

### Oligonucleotide Features.

The disclosure provides oligonucleotide dendrons that comprise an oligonucleotide stem linked to a plurality of oligonucleotide branches through a doubler moiety, a trebler moiety, or a combination thereof. Thus, in some embodiments, each oligonucleotide dendron has the ability to bind to a plurality of target polynucleotides having a sequence sufficiently complementary to the target polynucleotide to hybridize under the conditions being used. For example, if a specific polynucleotide is targeted, a single oligonucleotide dendron has the ability to bind to multiple copies of the same molecule. In some embodiments, methods are provided wherein the oligonucleotide dendron comprises identical oligonucleotide branches, i.e., each oligonucleotide branch has the same length and the same sequence. In other aspects, the oligonucleotide dendron comprises oligonucleotide branches that are not identical, i.e., at least one of the oligonucleotide branches of an oligonucleotide dendron differs from at least one other oligonucleotide branch of the oligonucleotide dendron in that it has a different length and/or a different sequence. Accordingly, in various aspects, a single oligonucleotide dendron may be used in a method to inhibit expression of more than one gene product. In some embodiments, one or more oligonucleotide branches of the oligonucleotide dendron is an inhibitory oligonucleotide as described herein. In some embodiments, the oligonucleotide stem and one or more oligonucleotide branches of the oligonucleotide dendron are immunostimulatory oligonucleotides as described herein. Thus, in various aspects, the disclosure provides methods of immune regulation.

In some embodiments, one or more oligonucleotide branches of the oligonucleotide dendron is an immunostimulatory oligonucleotide as described herein. In some embodiments, the oligonucleotide stem of the oligonucleotide dendron is an immunostimulatory oligonucleotide as described herein. In some embodiments, the oligonucleotide stem and one or more or all oligonucleotide branches of the oligonucleotide dendron is an immunostimulatory oligonucleotide as described herein.

### Methods

In various aspects and embodiments, oligonucleotide dendrons of the disclosure may be used in methods of inducing an immune response, immune regulation, and gene regulation.

Thus, the disclosure includes methods for eliciting an immune response in a subject in need thereof, comprising administering to the subject an effective amount of an antigenic composition comprising one or more of oligonucleotide dendrons as described herein. The immune response raised by the methods of the present disclosure generally includes an antibody response, preferably a neutralizing antibody response, antibody dependent cell-mediated cytotoxicity (ADCC), antibody cell-mediated phagocytosis (ADCP), complement dependent cytotoxicity (CDC), and T cell-mediated response such as CD4+, CD8+. The immune response generated by the oligonucleotide dendrons as disclosed herein generates an immune response that recognizes, and preferably ameliorates and/or neutralizes, a disorder (e.g., cancer, a viral infection) as described herein. Methods for assessing antibody responses after administration of an antigenic composition (immunization or vaccination) are known in the art and/or described herein. In some embodiments, the immune response comprises a T cell-mediated response (e.g., peptide-specific response such as a proliferative response or a cytokine response). In some embodiments, the immune response comprises both a B cell and a T cell response. Antigenic compositions can be administered in a number of suitable ways, such as intramuscular injection, subcutaneous injection, intradermal administration and mucosal administration such as oral or intranasal. Additional modes of administration include but are not limited to intravenous, intraperitoneal, intranasal administration, intra-vaginal, intra-rectal, and oral administration. A combination of different routes of administration in the immunized subject, for example intramuscular and intranasal administration at the same time, is also contemplated by the disclosure.

Administration can involve a single dose or a multiple dose schedule. Multiple doses may be used in a primary immunization schedule and/or in a booster immunization schedule. In general, the amount of oligonucleotide dendron in each dose of the antigenic composition is selected as an amount effective to induce an immune response in the subject, without causing significant, adverse side effects in the subject. Preferably the immune response elicited includes: neutralizing antibody response; antibody dependent cell-mediated cytotoxicity (ADCC); antibody cell-mediated phagocytosis (ADCP); complement dependent cytotoxicity (CDC); T cell-mediated response such as CD4+, CD8+, or a protective antibody response. Protective in this context does not necessarily require that the subject is completely protected against infection. A protective response is achieved when the subject is protected from developing symptoms of disease.

In any of the aspects or embodiments of the disclosure, an oligonucleotide dendron comprises an immunomodulatory (e.g., immunostimulatory, immunosuppressive) oligonucleotide. Toll-like receptors (TLRs) are a class of proteins, expressed in sentinel cells, that play a key role in regulation of innate immune system. The mammalian immune system uses two general strategies to combat infectious diseases. Pathogen exposure rapidly triggers an innate immune response that is characterized by the production of immunostimulatory cytokines, chemokines and polyreactive IgM antibodies. The innate immune system is activated by exposure to Pathogen Associated Molecular Patterns (PAMPs) that are expressed by a diverse group of infectious microorganisms. The recognition of PAMPs is mediated by members of the Toll-like family of receptors. TLR receptors, such as TLR 4, TLR 8 and TLR 9 that respond to specific oligonucleotide are located inside special intracellular compartments, called endosomes. The mechanism of modulation of, for example and without limitation, TLR 4, TLR 8 and TLR 9 receptors, is based on DNA-protein interactions.

Synthetic immunostimulatory oligonucleotides that contain CpG motifs that are similar to those found in bacterial DNA stimulate a similar response of the TLR receptors. Therefore, immunomodulatory oligonucleotides have various potential therapeutic uses, including treatment of immune deficiency and cancer. Thus, in some embodiments, an oligonucleotide dendron of the disclosure comprises one or more or all oligonucleotide branches that is an immunostimulatory oligonucleotide (e.g., a TLR agonist). In some embodiments, an oligonucleotide dendron of the disclosure comprises an oligonucleotide stem that is an immunostimulatory oligonucleotide (e.g., a TLR agonist). In some embodiments, an oligonucleotide dendron of the disclosure comprises an oligonucleotide stem and one or more or all oligonucleotide branches that are immunostimulatory oligonucleotides (e.g., TLR agonists). In some embodiments, an oligonucleotide dendron of the disclosure comprises one or more or all oligonucleotide branches that is an immunosuppressive oligonucleotide (e.g., a TLR antagonist). In some embodiments, an oligonucleotide dendron of the disclosure comprises an oligonucleotide stem that is an immunosuppressive oligonucleotide (e.g., a TLR antagonist). In some embodiments, an oligonucleotide dendron of the disclosure comprises an oligonucleotide dendron comprising an oligonucleotide stem and one or more or all oligonucleotide branches that are immunosuppressive oligonucleotides (e.g., TLR antagonists). In some embodiments, the immunostimulatory oligonucleotide is a double-stranded DNA (dsDNA).

In further embodiments, down regulation of the immune system involves knocking down the gene responsible for the expression of the Toll-like receptor. This antisense approach involves use of an oligonucleotide dendron of the disclosure to knock down the expression of any toll-like protein.

Accordingly, in some embodiments, methods of utilizing oligonucleotide dendrons as described herein for modulating toll-like receptors are disclosed. The method either up-regulates or down-regulates the Toll-like-receptor activity through the use of a TLR agonist or a TLR antagonist, respectively. The method comprises contacting a cell having a toll-like receptor with one or a plurality of oligonucleotide dendrons of the disclosure, thereby modulating the activity and/or the expression of the toll-like receptor. The toll-like receptors modulated include one or more of toll-like receptor 1, toll-like receptor 2, toll-like receptor 3, toll-like receptor 4, toll-like receptor 5, toll-like receptor 6, toll-like receptor 7, toll-like receptor 8, toll-like receptor 9, toll-like receptor 10, toll-like receptor 11, toll-like receptor 12, and/or toll-like receptor 13.

Thus, it is contemplated that in any of the aspects or embodiments of the disclosure, one or a plurality of oligonucleotide dendrons as disclosed herein possesses the ability to regulate gene expression. Thus, in some embodiments, an oligonucleotide dendron of the disclosure comprises an oligonucleotide (e.g., oligonucleotide stem and/or one or more oligonucleotide branches) having gene regulatory activity (e.g., inhibition of target gene expression or target cell recognition). In some aspects, the disclosure provides a method of inhibiting expression of a gene comprising the step of hybridizing a polynucleotide encoding the gene product with an oligonucleotide dendron or composition of the disclosure, wherein hybridizing between the polynucleotide and the oligonucleotide stem and/or one or more or all of the plurality of oligonucleotide branches of the oligonucleotide dendron occurs over a length of the polynucleotide with a degree of complementarity sufficient to inhibit expression of the gene product. In some embodiments, the hybridizing between the polynucleotide and the oligonucleotide stem and/or one or more or all of the plurality of oligonucleotide branches of the oligonucleotide dendron occurs in the absence of a transfection reagent (e.g., lipofectamine (Thermo Fisher)). In some embodiments, an oligonucleotide dendron of the disclosure comprises an oligonucleotide stem that is an inhibitory oligonucleotide as described herein. In some embodiments, an oligonucleotide dendron of the disclosure comprises one or more oligonucleotide branches that is an inhibitory oligonucleotide as described herein. In some embodiments, an oligonucleotide dendron of the disclosure comprises an oligonucleotide stem and one or more oligonucleotide branches that are inhibitory oligonucleotides. Accordingly, in some embodiments the disclosure provides methods for inhibiting gene product expression, and such methods include those wherein expression of a target gene product is inhibited by about or at least about 5%, about or at least about 10%, about or at least about 15%, about or at least about 20%, about or at least about 25%, about or at least about 30%, about or at least about 35%, about or at least about 40%, about or at least about 45%, about or at least about 50%, about or at least about 55%, about or at least about 60%, about or at least about 65%, about or at least about 70%, about or at least about 75%, about or at least about 80%, about or at least about 85%, about or at least about 90%, about or at least about 95%, about or at least about 96%, about or at least about 97%, about or at least about 98%, about or at least about 99%, or 100% compared to gene product expression in the absence of the oligonucleotide dendron. In other words, methods provided embrace those which results in essentially any degree of inhibition of expression of a target gene product. The disclosure contemplates inhibition of any gene product. In some embodiments, the target gene product is signal transducer and activator of transcription (STAT) 3 (STAT3). In further examples, the target gene product is a tumor-associated antigen. In some embodiments, the target gene product is human epidermal growth factor receptor 2 (HER2). In some examples, the target gene product is an enzyme (e.g., luciferase).

The degree of inhibition is determined in vivo from a body fluid sample or from a biopsy sample or by imaging techniques well known in the art. Alternatively, the degree of inhibition is determined in a cell culture assay, generally as a predictable measure of a degree of inhibition that can be expected in vivo resulting from use of a specific type of oligonucleotide dendron and a specific oligonucleotide. In various aspects, the methods include use of an oligonucleotide branch sufficiently complementary to a target polynucleotide as described herein. Accordingly, methods of utilizing an oligonucleotide dendron of the disclosure in gene regulation therapy are provided. This method comprises the step of hybridizing a polynucleotide encoding the gene with one or more oligonucleotides (e.g., an oligonucleotide stem and/or one or more or all of the oligonucleotide branches) complementary to all or a portion of the polynucleotide, wherein hybridizing between the polynucleotide and the oligonucleotide occurs over a length of the polynucleotide with a degree of complementarity sufficient to inhibit expression of the gene product. The inhibition of gene expression may occur in vivo or in vitro.

The inhibitory oligonucleotide utilized in the methods of the disclosure is either RNA, DNA, or a modified form thereof. In various embodiments, the inhibitory oligonucleotide is antisense DNA, small interfering RNA (siRNA), an aptamer, a short hairpin RNA (shRNA), a DNAzyme, or an aptazyme.

The following examples are given merely to illustrate the present disclosure and not in any way to limit its scope.

All references, patents, and patent applications disclosed herein are incorporated by reference with respect to the subject matter for which each is cited, which in some cases may encompass the entirety of the document.

### EXAMPLES

In the following Examples, the chemical modularity of the oligonucleotide dendron was leveraged to study structure-function relationships that dictate molecular vaccine efficacy, particularly regarding the delivery of immune-activating oligonucleotide sequences and antigenic peptides on a single chemical construct. How adjuvant and antigen placement and number impact dendron cellular uptake and immune activation was investigated. These parameters also played a significant role in raising a potent and specific immune response against target cancer cells, both in vitro and in vivo. Specifically, it was investigated how adjuvant placement and attachment chemistry impact dendron uptake and adjuvant potency. By conjugating an antigenic peptide, derived from cervical cancer due to human papillomavirus (HPV), to different positions on the dendron architecture, this system was used to investigate how antigen placement impacts vaccine cellular uptake and immune activation. Finally, these structures were utilized to train immune cells to target and kill cancer cells, both in vitro and in vivo, demonstrating that vaccine efficacy is highly structure-dependent. Taken together, the data establish the oligonucleotide (e.g., DNA) dendron as a powerful tool to study the fundamental structure-function relationships that govern cancer vaccine efficacy. Furthermore, the observation that DNA dendrons themselves can behave as potent vaccines has important implications in the development of new classes of cancer immunotherapeutics.

By gaining this structural understanding of molecular vaccines, oligonucleotide dendrons successfully treated a mouse cervical human papillomavirus TC-1 cancer model, in vivo, where the vaccine structure defined its efficacy; the top performing design effectively reduced tumor burden (<100 mm³ through day 30) and maintained 100% survival throughout the duration of the study (44 days after tumor inoculation).

### Example 1

### Materials and Methods

### Materials:

TC-1 cells were kindly provided by Dr. Bin Zhang. All animals were used in accordance with approved protocols of the Institutional Animal Care and Use Committee of Northwestern University. Animals (female C57BL/6, 8-12 weeks old) were obtained from Jackson Laboratories.

### Synthesis of Oligonucleotides:

Oligonucleotide synthesis, purification, and characterization was conducted as reported previously.²⁷ Briefly, reagents and solid-phase supports were purchased from Glen Research. Linear oligonucleotides were synthesized using a MerMade 12 synthesizer (Bio Automation) on controlled pore glass (CPG) beads (Universal UnyLinker Support (1000 Å)), using conditions recommended by the manufacturer. DNA dendrons were synthesized using a modified coupling protocol reported previously.27 Specifically, they were synthesized using an ABI synthesizer on a dT CPG (2000 Å) with 2× phosphoramidite concentration for bases on the branches of the dendron. Linear oligonucleotides were purified using reverse-phase high-performance liquid chromatography (RP-HPLC; Agilent), while the DNA dendrons were purified using denaturing polyacrylamide gel electrophoresis (PAGE). The samples were characterized using matrix-assisted laser desorption/ionization-time of flight (MALDI-TOF; AutoFlex-III, Bruker) mass spectrometry (matrix: dihydroxyacetone phosphate). A complete list of synthesized oligonucleotides is shown in Tables 1-2. The concentrations of DNA dendrons and DNA-containing templates were determined by measuring the solution absorbance at A=260 nm (Cary 5000 UV-vis spectrophotometer, Varian) and using the extinction coefficients calculated by the OligoAnalyzer tool (Integrated DNA Technologies).

TABLE 1
DNA Design

| | | Calc'd | Exp't | SEQ |
|---|---|---|---|---|
| Name | DNA Sequence (5′-3′) | MW | MW | ID NO: |
| 1826 CpG | TCC ATG ACG TTC CTG ACG TT | 6059 | 6057 | 1 |
| Dn1a | TTT TTT TTT T D Tr Sp Cy3 TCC ATG ACG TTC CTG ACG TT | 26643.94 | 26701 | 2 |
| Dn1aH | TTT TTT TTT T D Tr Sp Cy3 AAC GTC AGG AAC GTC ATG GA | 26760.04 | 26767 | 3 |
| Dn6a | TCC ATG ACG TTC CTG ACG TT D Tr Sp Cy3 TTT TTT TTT T | 42038.84 | 42015 | 4 |
| Dn6aH | AAC GTC AGG AAC GTC ATG GA D Tr Sp Cy3 TTT TTT TTT T | 42735.44 | 42775 | 5 |
| Dn1 | TTT TTT TTT T D Tr Sp Cy3 TCC ATG ACG TTC CTG ACG T AmdT | 26798.14 | 26803 | 6 |
| Dn6m | TTT TT AmdT TTT T D Tr Sp Cy3 TCC ATG ACG TTC CTG ACG TT | 27569.24 | 27588 | 7 |
| Dn6e | AmdT TTT TTT TTT D Tr Sp Cy3 TCC ATG ACG TTC CTG ACG TT | 27569.24 | 27553 | 8 |

Acronyms for phosphoramidites:
Sp: 18-O-Dimethoxytritylhexaethyleneglycol, 1-[(2-cyanoethyl)-(N,N-diisopropyl)]-phosphoramidite (Spacer phosphoramidite 18)
D: 1,3-bis-[5-(4,4′-dimethoxytrityloxy)pentylamido]propyl-2-[(2-cyanoethyl)-(N,N-diisopropyl)]phosphoramidite (Symmetric doubler phosphoramidite)
Tr: Tris-2,2,2-[3-(4,4′-dimethoxytrityloxy) propyloxymethyl]methyleneoxypropyl-[(2-cyanoethyl)-(N,Ndiisopropyl)]-phosphoramidite (Long trebler phosphoramidite)
Cy3: 1-[3-(4-monomethoxytrityloxy) propyl]-1′-[3-[(2-cyanoethyl)-(N,N-diisopropyl) phosphoramidityl] propyl]-3,3,3′,3′-tetramethylindocarbocyanine chloride (Cyanine 3 phosphoramidite)
AmdT: 5′-Dimethoxytrityl-5-[N-(trifluoroacetylaminohexyl)-3-acrylimido]-2′-deoxyUridine,3′-[(2-cyanoethyl)-(N,N-diisopropyl)]-phosphoramidite (Amino Modifier C6 dT)

TABLE 2
Peptide Sequence Information

| Name | Amino Acid Sequence (N—C) | MW |
|---|---|---|
| E6⁴⁹⁻⁵⁸ (V10C) | VYDFAFRDLC (SEQ ID NO: 9) | 1248 |

Peptides were obtained through GenScript at >95% purity.

### Cellular Uptake and Immune Activation of DNA Dendrons and Conjugates:

Bone marrow-derived dendritic cells (BMDCs) were harvested from C57BL/6 mice. Cells were flushed from inside the bone, and then collected by centrifugation (1200 rpm, 5 min). Cells were lysed using 2 mL of ACK lysing buffer for 4 min at room temperature to selectively lyse red blood cells. The remaining cells were washed once with Phosphate Buffered Saline (PBS) and cultured by incubating in dishes with Gibco Roswell Park Memorial Institute 1640 Medium (RPMI) containing 10% heat inactivated Fetal Bovine Serum (HI-FBS), 1% penicillin/streptomycin (P/S), and 40 ng/ml Granulocyte-macrophage colony-stimulating factor (GM-CSF) for 5 days. Media was added after three days to maintain appropriate nutrients. Cells were collected from the plate and transferred to microtiter tubes prior to addition of treatment.

Cells described above were treated with DNA for 1 h at 50 nM or 250 nM, or for 15 h at 1000 nM (by DNA) and stored in a 37° C./5% CO₂ incubator for the specified amount of time. At the completion of the timepoint, cells were washed with PBS and spun at 1200 rpm for 5 min to remove supernatant, and incubated with fluorophore-conjugated antibodies (Fixable Live/Dead-DAPI, Invitrogen #L34969; CD11c—Alexa Fluor 647, Biolegend #117312 and BV421, Biolegend #117330; CD80—FITC, Biolegend #104706; CD86—PerCP cy5.5, Biolegend #105028) for 15 min at 4° C. Then, cells were washed with PBS, resuspended in fixation buffer (Biolegend #420801), and incubated at 4° C. for at least 15 min or until analyzed by flow cytometry. All experiments have been conducted in triplicate and with at least three experimental replicates.

### DNA Dendron Uptake Mechanism Experiments:

Dendron uptake mechanisms were tested by repeating the previously described uptake experiment in dendritic cells with minor modifications. The cells were pretreated with 50 μg/mL of fucoidan from fucus vesiculous (Sigma F8190), methyl-beta-cyclodextran (12.5 mg/mL), or at 4° C. for 30 min prior to DNA treatment. DNA was added to the cells at concentrations of either 50 nM and 250 nM (by DNA) and incubated for 1 h and 6 h at either 37° C./5% CO₂ or 4° C. At the completion of the timepoints, cells were washed with PBS and spun at 1200 rpm for 5 min to remove the supernatant, and incubated with fluorophore-conjugated antibodies (Fixable Live/Dead—Ultra-Violet, Invitrogen #L34961 and CD11c—Alexa Fluor 647, Biolegend #117312) for 15 min at 4° C. Then, cells were washed with PBS, resuspended in fixation buffer (Biolegend #420801), and incubated at 4° C. for at least 15 min or until analyzed by flow cytometry (up to 3 days after collection).

### Lymph Node Uptake:

Female C57BL/6 mice (8-12 weeks old) were administered a single subcutaneous injection into the abdomen. Treatment dose was maintained at 6 nmol. After 4 h, mice were euthanized and the skin containing the lymph nodes was resected. Fluorescence was assessed using an IVIS 200 Spectrum (PerkinElmer) in vivo imaging system with a narrow band excitation of 535 and emission of 580. Quantitative analysis was performed using Living Image software.

### Ex Vivo T Cell Killing:

Murine PBMCs (BioIVT) were thawed from storage in liquid nitrogen and 1×10⁴ cells were added to a 96-well round bottom plate in a 450 μl volume. Treatment was added to each well in a 450 μl volume, and cells were left in a 37° C. in a 5% CO₂ incubator for 48 h. The following procedures were modified from a previously established protocol²³. Briefly, CD8+ T cells were magnetically isolated using a murine CD8a Positive Selection Kit II (StemCell Technologies). Concurrently, TC-1 cells were collected from a passage and were stained with efluor450 (eBioscience) following the manufacturer's protocol. TC-1 cells were counted after staining and were plated in a new 96-well round bottom plate with 5000 cells per well in a volume of 100 μL. Cells recovered while T cells were isolated and counted. T cell concentration was adjusted through resuspension in RPMI media containing 10% HI-FBS and 1% P/S. T cells were added to be at a final ratio of 5:1 with TC-1 target cells in the 96-well plate. Cells were co-cultured together for approximately 24 h in a 37° C./5% CO₂ incubator. After the incubation time, all cells were collected into microtiter tubes using trypsin to detach adherent cells and media to neutralize the trypsin. Samples were washed once with PBS, centrifuged at 1200 rpm for 5 min, aspirated, and resuspended in a solution of 100 μL Annexin V binding buffer (BioLegend) containing 0.5 μL each of 7-AAD (Fisher, 50169259) and Annexin V (BioLegend, 640906). This staining solution was left with cells for 15 min at room temperature in the dark prior to flow cytometry.

### In Vivo Therapeutic Efficacy:

Female C57BL/6 mice aged 8-12 weeks (Jackson Laboratory) were inoculated with 2×10⁵ TC-1 tumor cells subcutaneously into the right flank and were allowed to grow to approximately 50 mm³ (7 days) prior to treatment. Treatments were administered at a dose of 6 nmol (60 μM in 100 μL volume) by subcutaneous injection into the abdomen once per week, following the schedule provided. Tumor growth was measured every 2-3 days and volume was calculated using the following equation: tumor volume=length×width²×0.5. Animals were euthanized when tumor volumes reached 1,500 mm³ or when animal health necessitated them to be sacrificed for humane reasons.

### Results

### Design, Uptake, and Efficacy of Immune-Stimulating DNA Dendrons.

The adjuvant is a key component of a vaccine because it leads to immune activation and cellular processing of the antigen target.²⁹ Previous work has shown that increasing the valency of DNA dendrons (and thereby the number of adjuvant sequences) leads to increased immune activation;³⁰ however, important structural questions, such as the effects of steric hinderance and DNA hybridization are yet to be explored and can have a significant impact on adjuvant function (vide infra). When considering multivalent adjuvant delivery and efficacy, steric hinderance can play a crucial role in determining adjuvant potency.

To that end, four different six-branched DNA dendrons were designed that contain adjuvant CpG (cytosine-phosphate-guanine) DNA sequences that behave as potent toll-like receptor 9 (TLR-9) agonists: 1) a T10 sequence stem that branches into six CpG sequences (termed Dn6a, for dendron with 6 adjuvant strands); 2) a T10 sequence stem that branches into six CpG complementary sequences that hybridize the CpG sequences (termed Dn6aH, for dendron with 6 adjuvant strands hybridized); 3) a CpG sequence stem that branches into six T10 sequences (termed Dn1a, for dendron with 1 adjuvant strand); and 4) a CpG complement strand that can hybridize the CpG strand and branches into six T10 sequences (termed Dn1aH, for dendron with 1 adjuvant strand hybridized) (FIG. 1A). T10 sequences were chosen due to synthetic ease and lack of secondary structure formation³⁹⁻⁴¹. These designs allowed for the probing of steric effects in detail. By comparing data from Dn6a and Dn1a, we can deduce whether multivalency improves or impairs adjuvant function as a result of steric hindrance from dense DNA packing on the branches. Comparing hybridized (Dn6aH and Dn1aH) and unhybridized structures (Dn6a and Dn1a) reveals whether the dendron molecule itself sterically inhibits proper TLR-9 binding and processing and if a supramolecular dendron design is therefore necessary for potent activation.

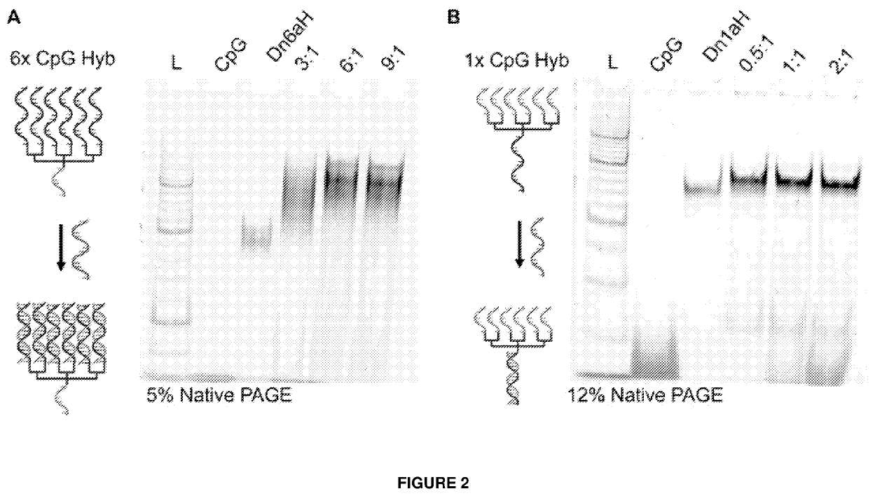

For in vitro measurements via flow cytometry, all DNA dendrons were synthesized with a cyanine 3 (Cy3) fluorescent dye in the middle of the structure, which not only allowed for a quantitative assessment of dendron uptake, but also allowed for the facile tracking of the dendron throughout its synthesis and purification. The DNA dendrons were synthesized through solid-phase automated synthesis, purified by polyacrylamide gel electrophoresis (PAGE), and characterized by PAGE and Matrix Assisted Laser Desorption/Ionization Time of Flight Mass Spectrometry (MALDI-TOF MS) (Table 1). To form the hybridized structures, CpG strands were mixed with Dn6aH and Dn1aH at stoichiometric amounts, annealed from 90 to 20° C., over 1 h, and characterized by native PAGE (FIG. 2).

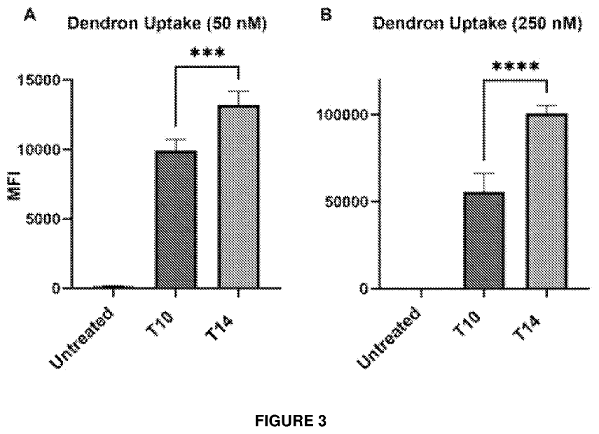

First, it was determined how the structure of each dendron impacted the cellular uptake efficiency. To test this, murine bone-marrow-derived dendritic cells (BMDCs) were treated with 250 nM fluorophore-labeled DNA dendrons (1:1 ratio of dendron molecule to fluorescent tag) for 1 h in serum-containing media. Cellular uptake was assessed by measuring the median fluorescence intensity (MFI) of the Cy3 fluorophore, in treated cells, via flow cytometry. After 1 h, it was observed that Dn6aH and Dn1aH had significantly lower cellular uptake efficiencies compared to Dn6a and Dn1a (FIG. 1B), presumably due to a decrease in DNA-scavenger receptor A recognition. The non-hybridized structures had MFIs over two orders of magnitude greater than those of the hybridized structures, indicating that dendron uptake is maximized when the branches and stems are single-stranded DNA. The Dn6a-treated cells also had 1.2-fold greater MFI than those treated with the Dn1a. This observation can be attributed to the fact that longer DNA sequences can lead to increased uptake efficiencies (FIG. 3).²⁷ This experiment was repeated with 1 uM of fluorescently labeled DNA dendron and an incubation time of 15 h. Even at this longer timepoint, it was observed that the non-hybridized structures enter cells in greater quantity than the hybridized forms. Specifically, the Dn6aH structure suffers from low uptake indicating that the double-stranded dendron branches significantly inhibit cellular uptake (FIG. 1C).

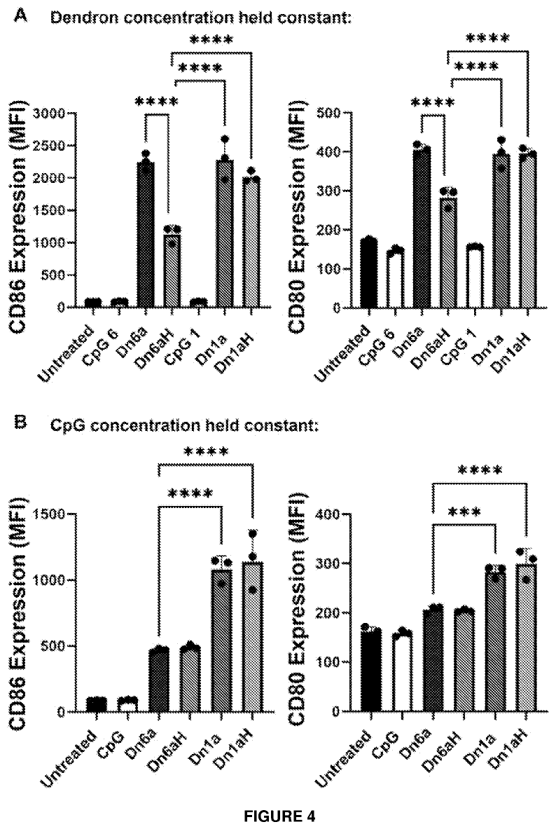

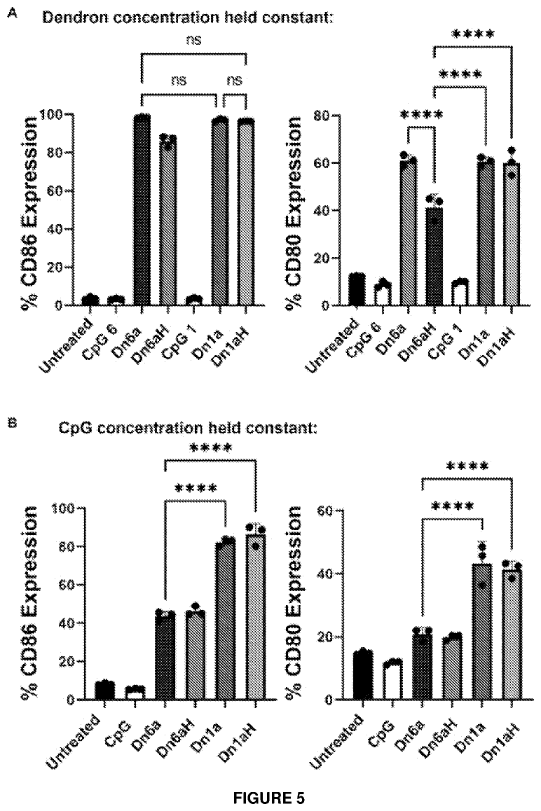

Next, BMDCs were treated with either linear CpG controls or one of the four dendron designs and immune activation was measured after a 15 h incubation time. Upon TLR-9 activation by CpG adjuvant DNA, immune co-stimulatory markers, such as cluster of differentiation (CD) 86 and CD80, became upregulated and presented on the dendritic cell surface.³¹ Which dendrons elicit the greatest immune response was assessed by measuring the amount of CD80 and CD86 expressed. To address the fact that the Dn6a and Dn6aH structures deliver 6-fold more CpG per dendron than the Dn1a and Dn1aH structures, this experiment was conducted in two different ways. First, cells were treated such that the total dendron concentration was held constant, meaning that the Dn6a- and Dn6aH-treated cells received 6-fold more CpG than the Dn1a- and Dn1aH-treated cells but that the amount of dendron delivered was the same for each group (FIG. 4B). Due to the two different CpG concentrations between the Dn1a and Dn6a structures, two linear CpG controls were used to match those concentrations (CpG 1, to match Dn1a and Dn1aH, and CpG 6, to match Dn6a and Dn6aH). Expectedly, the linear CpG controls at either concentration had negligible cellular uptake and immune activation compared to the dendron treatment groups due to the rapid degradation and transport of linear DNA sequences. For cells treated with DNA dendrons, trends were observed in CD86 and CD80 expression that matched the uptake profile of the different groups—the Dn6a, Dn1a, and Dn1aH had no significant differences in CD86 and CD80 expression, while the Dn6aH structure produced significantly less CD86 and CD80 expression amongst murine CD11c+ dendritic cells. This trend held when the percent of cells positive for CD86 and CD80 expression was measured. Specifically, Dn6a, Dna, and Dn1aH induced CD86 expression in nearly 100% of cells, while the Dn6aH structure induced expression in only 80% of cells (FIG. 5). Expression of CD80 followed a similar trend. These observations can be attributed to the lower cellular uptake of the Dn6aH, as discussed above. Furthermore, these results indicated that although the Dn6a dendron delivered 6-fold more CpG than the Dn1a and Dn1aH dendrons, the total immune activation was nearly identical.

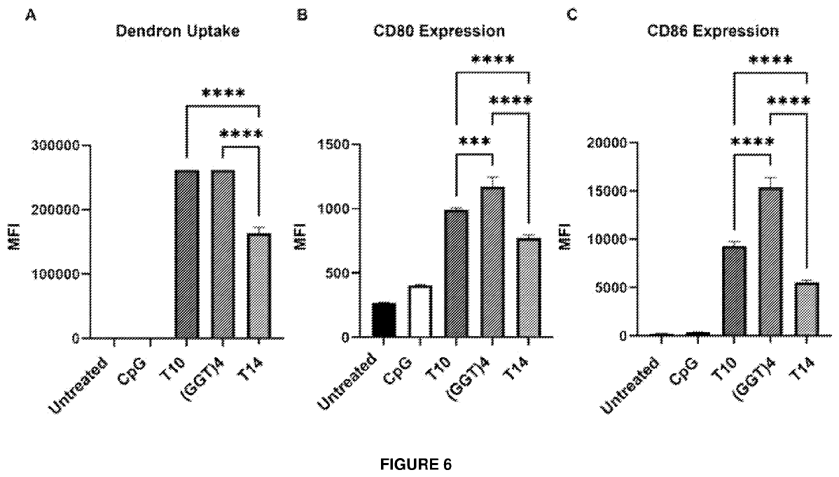

To further investigate this observation, cells were treated such that the CpG concentration was held constant across treatment groups. Therefore, cells treated with Dn6a and Dn6aH received ⅙th the amount of dendron molecule as those treated with Dn1a and Dn1aH (FIG. 4B, FIG. 5B). Interestingly, when the CpG concentration was held constant, a different trend in immune activation emerged. The Dn6a and Dn6aH structures resulted in significantly lower immune activation, whereby CD86 and CD80 levels were reduced by approximately 50% and 33% compared to the Dn1a and Dna1H responses, respectively (FIG. 4B). These data indicated that although the Dn6a dendron presented CpG multivalently, this multivalency did not lead to increased CpG processing and cell activation. This suggested that dendron valency does not impart the proper spatial requirements necessary for multivalent interactions with TLR-9.³² Taken together, these data revealed that the Dn1a and Dn1aH designs are the most effective for adjuvant delivery. Due to synthetic ease and molecular precision, the non-hybridized Dn1a structure was used in subsequent experiments. Moreover, since the branches of the DNA dendron are no longer used as the CpG sequence, they can be leveraged as orthogonal handles to improve cellular uptake and immune activation (i.e., G-rich sequences) (FIG. 6) or as secondary functional sequences (e.g., alternative TLR agonist sequences).

### Antigen Conjugation and its Effects on Immune Activation.

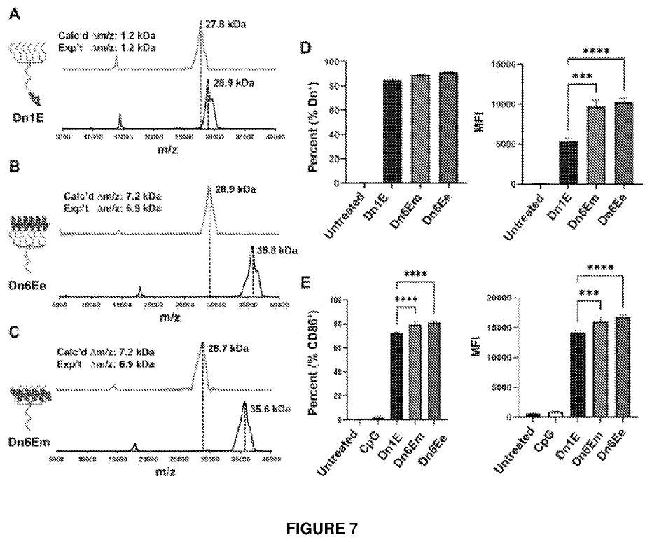

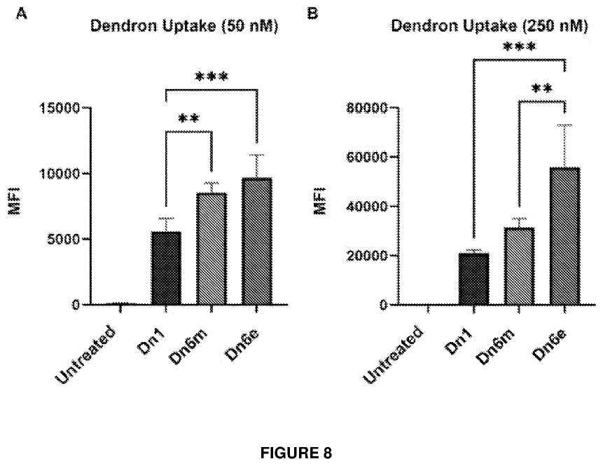

Having established design rules for dendron-mediated adjuvant delivery, it was next sought to understand how the placement of an antigenic peptide affects dendron vaccine efficacy. It was hypothesized that antigen placement on the dendron would significantly impact dendron uptake and overall vaccine efficacy due to the hydrophobic nature of many peptide antigens.³³ It was further hypothesized that the antigen/adjuvant ratio would significantly affect vaccine efficacy.²⁵ To begin exploring the impact of antigen placement, three dendrons were designed based on the Dn1a architecture. The first contained a primary amine on the 3′ end of the dendron stem such that a single antigen could be added to each structure (Dn1E, dendron with 1 epitope, FIG. 7A). The second design included primary amines on the 5′ end of the dendron, thereby producing dendrons with 6 peptides on the ends of the branches (1 peptide/branch) (Dn6Ee, dendron with 6 epitopes on the end, FIG. 7B. To test whether peptide conjugation to the 5′ end impacted dendron uptake (due to a potential loss of scavenger receptor A binding interactions), the third design had primary amines located in the middle of each branch, enabling the conjugation of six antigen molecules without blocking the multivalent DNA branches (Dn6Em, dendron with 6 epitopes in the middle, FIG. 7C. Interestingly, these dendrons on their own, without conjugated antigens, had distinct uptake properties. Specifically, the dendrons with more primary amine functional groups achieved nearly two-fold increases in cellular uptake (FIG. 8). This result was likely due to the positive charge of the primary amine under biological conditions, which can facilitate cellular uptake.³⁴ Nevertheless, upon peptide conjugation the amine is converted to an amide and the positive charge is negated.

The peptide antigen E649-58 (V10C, sequence: VYDFAFRDLC (SEQ ID NO: 9), mw=1.25 kDa, Table 2 derived from the E6 protein, which is found in cervical cancer cells caused by human papillomavirus (HPV) was utilized. The peptide contains a single terminal cysteine amino acid which was conjugated to the dendron through a reducible disulfide bond. Specifically, the heterobifunctional crosslinker, succinimidyl 3-(2-pyridyldithio) propionate (SPDP), was used, which contains an activated ester on one end, employed for primary amine functionalization, and a pyridyldithiol group on the other end, used for sulfhydryl functionalization. First, the activated ester was reacted with the primary amines on the DNA dendrons, producing pyridyldithiol functionalized dendrons. Second, the E6 peptide was reacted with the pyridyldithiol to form peptide conjugates. All conjugates were purified by PAGE. MALDI-TOF MS results indicate that for each of the dendron designs, the expected product was synthesized and purified successfully because the expected mass shifts between unconjugated and conjugated DNA dendrons were observed (FIG. 7A-C) top and bottom, respectively). Specifically, the Dn1E structure showed a mass shift of 1.2 kDa, while the Dn6E structures show a mass shift of 6.9 kDa, closely matching the expected changes in mass for dendrons conjugated with one or six peptides, respectively.

After synthesizing the dendron peptide conjugates, how peptide placement affects cellular uptake was investigated. It was hypothesized that the increased hydrophobicity of the conjugated peptides would facilitate uptake and lead to increased uptake frequency and fluorescence intensity. Using flow cytometry, it was found that a similar percentage of cells were positive for each of the structures (% Dn+) (FIG. 3D, left); however, cells treated with dendrons with a greater number of conjugated peptides (Dn6Em and Dn6Ee) exhibited a nearly 2-fold higher MFI compared to those treated with Dn1E (FIG. 7D, right), demonstrating that more Dn6Em and Dn6Ee entered each cell when compared to the mono-conjugated Dn1E dendron.

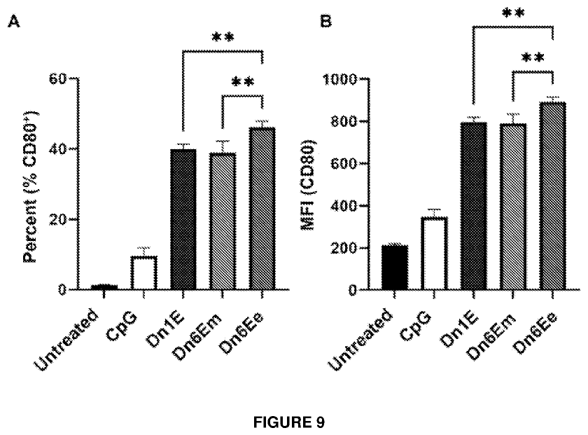

A similar trend was observed when measuring immune activation via CD86 expression (FIG. 7E). A statistically significant increase in the frequency of cells expressing CD86 (% CD86+) was observed when Dn6Em or Dn6Ee was used when compared to Dn1E (75% to 80%). When measuring the amount of CD86 expressed (MFI), a similar trend as seen in the uptake experiments was observed, wherein the Dn6E structures led to the expression of more CD86 than the Dn1E dendron (FIG. 7E, right). A similar trend was also observed when measuring the CD80 costimulatory marker (FIG. 9). These results confirmed the hypothesis that increasing the number of peptides attached to the dendron leads to increased cellular uptake and immune activation. Due to the similarity in uptake and activity for the Dn6Em and Dn6Ee constructs, it was chosen to continue experimenting with the Dn6Em structure for synthetic reasons, finding that Dn6Em peptide conjugation consistently had a 30% yield while the Dn6Ee structure had a 15% yield.

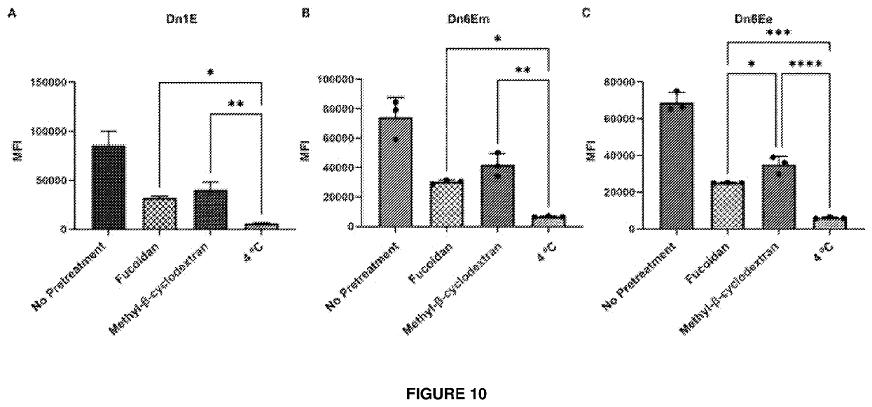

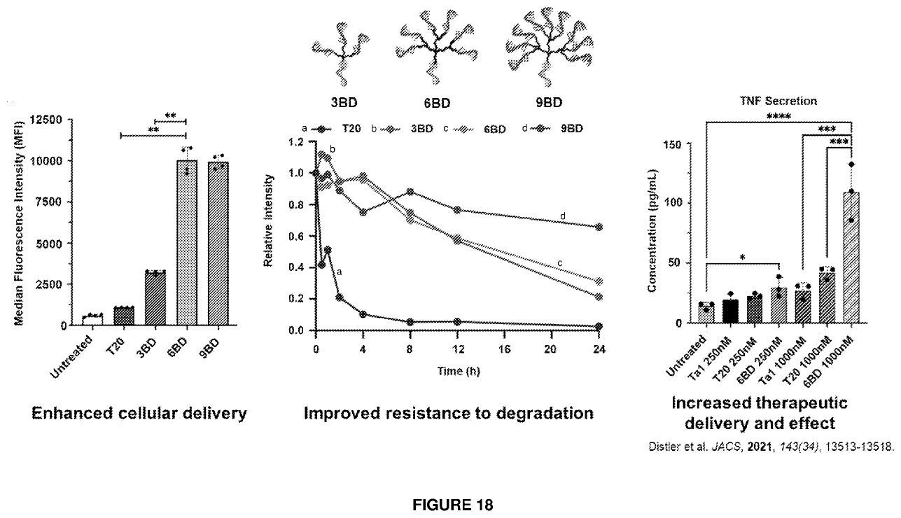

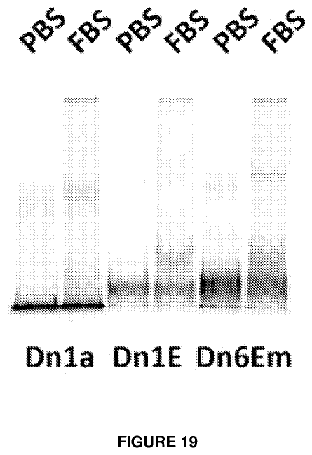

The uptake mechanism of these materials was explored by selectively inhibiting cellular uptake pathways (FIG. 10). Specifically, cells were pre-treated with either fucoidan (a scavenger receptor A inhibitor), methyl-β-cyclodextrin (cholesterol/lipid raft depletion), or incubated at 4° C. (which inhibits all active transport mechanisms). It was observed that inhibiting active transport through incubation at 4° C. resulted in the greatest decrease in uptake for each of the peptide conjugates, indicating that these peptides were not facilitating significant passive transport across the cell membranes. After treatment with methyl-β-cyclodextrin, which abstracts hydrophobic cholesterol and lipid rafts from the cell surface, observed a decrease in cellular uptake was observed, which is likely due to decreased interactions between hydrophobic peptides and hydrophobic components of the cell membrane. Finally, we observed that by inhibiting scavenger receptor A mediated endocytosis using fucoidan, the most significant drop (at least 50% in each treatment group) in uptake was observed, indicating that the primary pathway of dendron uptake was through scavenger receptor A, regardless of where the antigenic peptide was located on the dendron. It was also investigated whether peptide conjugation impacted the complexation of serum proteins to the dendron, which may explain the observed differences in cellular uptake efficiencies. To test this assertion, Dn1a, Dn1E, and Dn6Em were incubated in fetal bovine serum and subsequently analyzed by gel electrophoresis. Based on shifts in electrophoretic mobility, the results showed that there were mild differences in serum protein complexation to the dendrons depending on the amount of conjugated peptide (FIG. 19). These differences in protein complex formation may have contributed to the observed uptake properties. FIG. 18 shows that design parameters of oligonucleotide dendrons can be adjusted depending on a desired use such that the oligonucleotide dendron exhibits enhanced cellular delivery, improved resistance to degradation, and increased therapeutic delivery and effect.

### Investigating Vaccine Efficacy In Vitro and In Vivo.

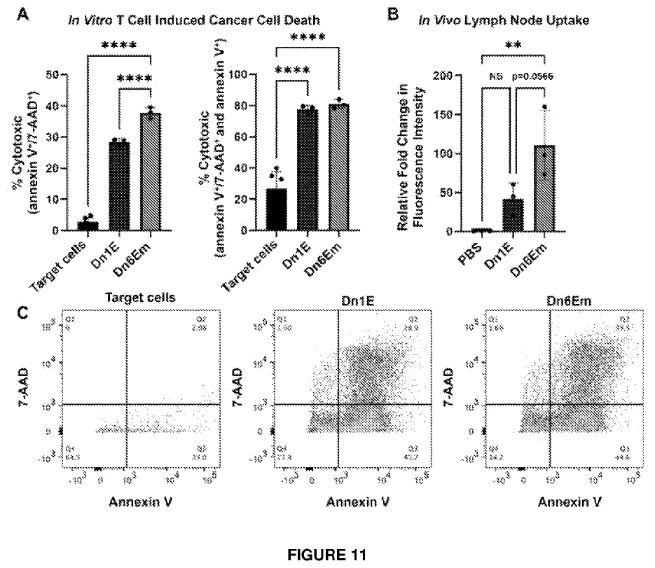

Before studying vaccine efficacy in vivo, it was first confirmed that the DNA dendron vaccines elicited a potent and specific immune response against cervical cancer cells. Mouse peripheral blood mononuclear cells (PBMCs) were treated with the different vaccines. The resulting raised T cells were isolated from the PBMCs and co-cultured separately with TC-1 mouse cervical cancer cells at a 10 to 1 ratio of T-cells to cancer cells. After incubation, cancer cell death was quantified by measuring 7-AAD (necrosis) and Annexin V (early apoptosis). It was observed that all tested DNA dendrons produced a potent immune response against the target cancer cells. Specifically, approximately 30% of the cells treated with Dn1E and approximately 40% of the cells treated with Dn6Em induced TC-1 expression of markers for both apoptosis and necrosis (FIG. 11A), left). However, if the target cells that were expressing only apoptotic markers were included (indicating that they are undergoing early apoptosis but not yet full necrosis), it was found that both vaccines killed approximately 80% of the target cells (FIG. 11A), right). Since early apoptosis occurs prior to necrosis, these results suggested that the vaccines produce an immune response at different rates. Indeed, the Dn6Em vaccine seemed to result in a potent immune response earlier than the Dn1E vaccine, leading to a greater number of necrotized target cells. This observation was likely due to the increase in antigen loading on the Dn6Em structure, as well as its increased cellular uptake. Representative scatter plots are provided in FIG. 11C.

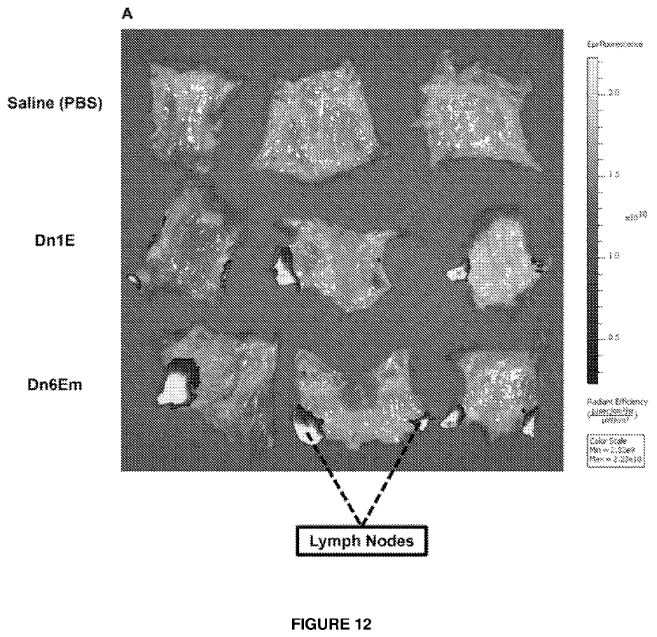

To further assess the potential in vivo efficacy of the dendron vaccines, how the dendron architecture facilitates effective uptake into the lymph nodes, the biological hub for immune cells responsible for producing immune responses, was studied.³⁵ Female C57BL/6 mice (n=3 per group) received either a saline control (PBS), Dn1E (6 nmol) or Dn6Em (6 nmol), subcutaneously. After 4 h, the mice were sacrificed, the lymph nodes were collected, and Cy3 fluorescence was quantified using an in vivo Imaging System (IVIS). It was observed that both vaccines localized to one or both of the proximal lymph nodes (FIG. 12) and that the Dn6Em structure was taken up by the lymph nodes more than the Dn1E vaccine (p=0.0566) (FIG. 11B). Negligible fluorescence was observed in the lymph nodes of the PBS treated mice, but importantly there was no statistically significant difference between the fluorescence intensities of PBS and Dn1E treated mice. This indicated that peptide location and amount on the dendron structure can significantly impact vaccine accumulation at the desired immune centers when delivered in vivo. Previous research has shown that the hydrophobicity of the therapeutic can significantly enhance lymph node uptake.³⁶⁻³⁸ Since the Dn6Em structure has 6-fold more peptides per dendron, those hydrophobic moieties may play an important role in mediating vaccine trafficking and uptake.

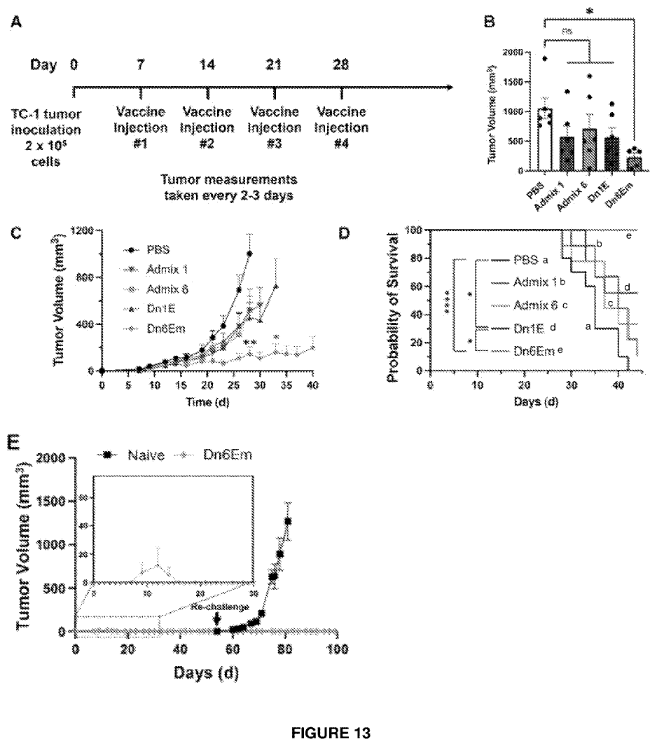

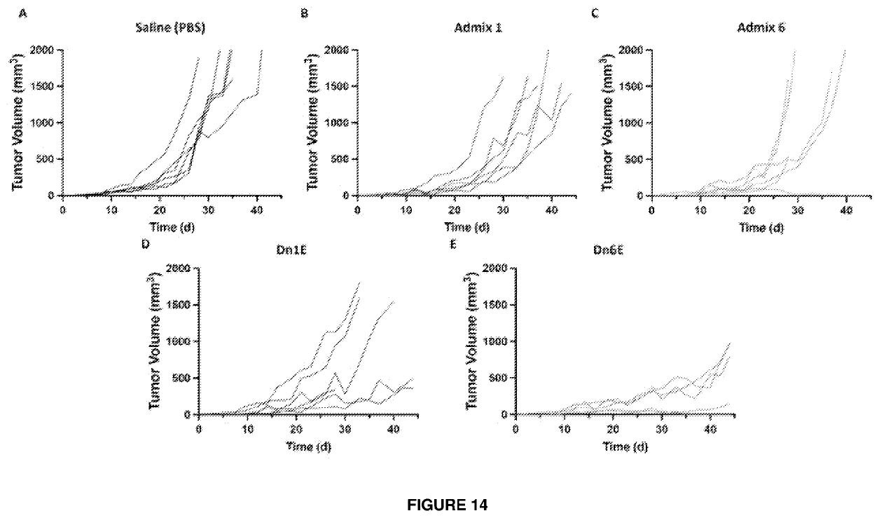

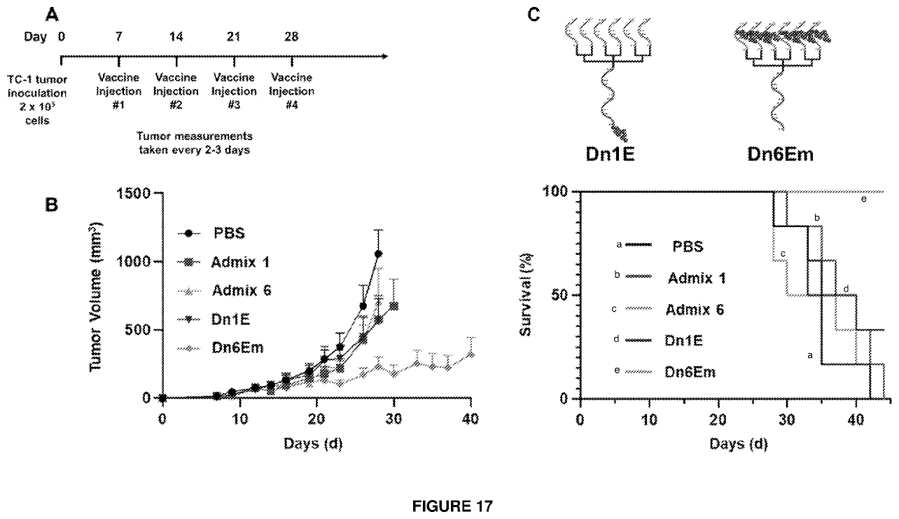

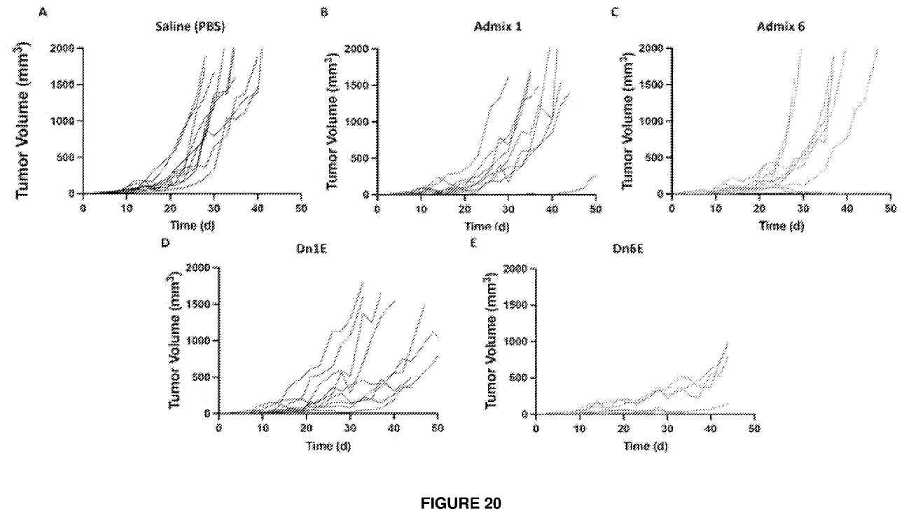

To evaluate the in vivo therapeutic efficacy, TC-1 tumor-bearing female mice (n=8-10) were treated with the different dendron architectures and tumor growth and animal survival was evaluated. TC-1 tumor cells (2×10⁵) were inoculated subcutaneously into the right flank of C57BL/6 mice and allowed to grow to approximately 50 mm³ before the first of four treatments (schedule provided in FIGS. 13A and 17A). Specifically, animals were treated once per week with Dn1E, Admix 1 (simple mixture corresponding to Dn1E adjuvant/antigen ratio), Dn6Em, Admix 6 (simple mixture corresponding to Dn6Em adjuvant/antigen ratio), or saline (PBS). Tumor growth was measured every 2-3 days while survival was quantified; animals were sacrificed when tumor burden reached 1500 mm³. Mice that were treated with Dn6Em exhibited potent suppression of tumor growth (FIGS. 13B, 17B, 13C, and 14). In fact, tumor growth inhibition was observed in 100% of animals treated with Dn6Em (on average <150 mm³ through day 30) and they were protected from death through the 44 day study as their tumors did not reach the 1500 mm³ cut-off during this timeframe (FIGS. 13D and 17C). In contrast, mice treated with the Dn1E dendron failed to exhibit a significant improvement in tumor burden, and only 56% survived to day 44. As expected, animals treated with saline all perished by the conclusion of the study (median survival—35 days), and animals treated with either of the admix controls were unable to mount a sufficient Immune response to fight off tumor burden effectively. Spider plots for all groups are provided in FIG. 14 and FIG. 20. This study illustrated the impact of precise structural changes to the dendron architecture on its ability to dramatically impact animal survival and tumor burden in vivo. At the conclusion of the study (after 44 days), mice with no measurable tumors were re-challenged with TC-1 tumor cells (FIG. 13E). Mouse health and tumor growth were monitored and measured for an additional 60 days. Naïve control mice experienced rapid increases in tumor size and required euthanasia by day 80 (36 days post challenge). Nevertheless, the two Dn6Em re-challenged mice were protected from the cancer cells, showing no signs of tumor growth through the conclusion of the study (Day 100). These results demonstrated that the oligonucleotide (e.g., DNA) dendron vaccines imparted an adaptive immune response capable of recognizing targets for several weeks post-immunization.

### Discussion

By leveraging the chemical addressability and structural modularity of the DNA dendron, this example explored concepts of rational vaccinology with molecular precision that underscored the importance of a well-defined vaccine architecture to elicit a potent immune response. Importantly, while these studies only explored a subset of all possible dendron valencies and adjuvant: antigen ratios, this investigation yielded several fundamental observations that are broadly applicable to any oligonucleotide (e.g., DNA) dendron-based vaccine. First, it was shown that the placement and attachment chemistry of the adjuvant CpG sequence within the dendron was critical for eliciting efficient cellular uptake and a potent immune response. Specifically, the DNA dendron that contained a single CpG sequence as the stem proved to be the most effective design for the delivery of functional adjuvant to cells, while the small size of the DNA dendron prevented any benefit from adjuvant multivalency and adjuvant hybridization significantly inhibited cellular uptake. Next, it was discovered that the number and placement of antigenic peptides along the dendron architecture affects dendron uptake, activation, and vaccine efficacy in vitro. Specifically, by increasing the amount of antigen conjugated to the dendron, cellular uptake and immune activation increased. It was also determined that the location of peptide placement was less important than the overall number in affecting vaccine properties. It was observed that the number of peptide antigens conjugated to the dendron impacted the rate at which dendron vaccines prompted an immune response against TC-1 ovarian cancer cells. Indeed, Dn6Em resulted in an approximately 30% increase in the amount of tumor cells that were both apoptotic and necrotic, likely a result of the dendrons increased uptake, immune activation, and antigen presentation. Translation of these vaccine designs in vivo was supported by the observed trend that increased peptide conjugation results in Dn6Em preferentially draining to murine lymph nodes to a greater extent than the mono-functionalized Dn1E architecture. Finally, it was observed that vaccine structure affects the overall immunogenicity of a cancer vaccine in vivo. Indeed, while highly immunogenic Dn6Em effectively reduced tumor burden (<100 mm³ through day 30), an additive mixture of its component adjuvant and antigen parts, as well as the Dn1E vaccine, failed to provide any significant benefit. It is expected that the conclusions drawn from this work are broadly applicable to oligonucleotide (e.g., DNA) dendron-based molecular vaccines. By utilizing the DNA dendron as a platform to study such concepts, structure-function relationships can be determined with molecular precision. Furthermore, although dendritic DNA architectures have been employed in the past for the delivery of therapeutic agents, many of these approaches are heterogenous and lack structural modularity. The DNA dendron-based vaccine disclosed herein comprises a single molecule, made using common oligonucleotide synthesis methods, with an easily modifiable structure. Thus, this work established the DNA dendron as a powerful tool for studying the fundamental structure-function relationships that govern cancer vaccine efficacy while simultaneously developing the DNA dendron as a potent platform for immunotherapy. DNA dendrons can be readily synthesized to have specific DNA sequences, lengths, and valences. By leveraging this high level of chemical control, DNA dendrons containing immune-stimulating CpG sequences were designed and synthesized. It was investigated how DNA dendron design impacts the effective delivery of functional CpG sequences and how to maximize cellular uptake and the resultant immune response in vitro. It was learned that having the CpG sequence as the dendron stem and poly-T sequences as the branches led to the most effective delivery of functional adjuvant. Nevertheless, the branch sequences have the potential to be further tuned to improve dendron uptake and function (e.g., G-rich sequences). By conjugating an antigenic peptide derived from HPV related cervical cancer to the dendron, a molecularly defined cancer vaccine was synthesized. It was discovered that peptide conjugation to different parts of the dendrons impacts dendron uptake and immune activation. Furthermore, these vaccines produced a robust and specific immune response against the cancer target, in vitro. Finally, it was uncovered that DNA dendron molecular vaccines elicit a robust and powerful anti-tumor response in vivo. Taken together, this work explored the structure-function relationships of DNA-dendron based vaccines in a molecularly defined manner. Due to the modularity of this molecular vaccine, these design rules can be applied to develop vaccines that treat a variety of cancers. These structure-function relationships can also be applied more broadly to develop related nanotherapeutic systems comprised of nucleic acids and other biomolecules.

### Example 2

### Materials.

Reagents and solid-phase supports were purchased from Glen Research. The A-431 (CRL-1555; human epidermoid carcinoma) cell line was purchased from American Type Culture Collection (ATCC).

### Synthesis of ASO Dendrons.

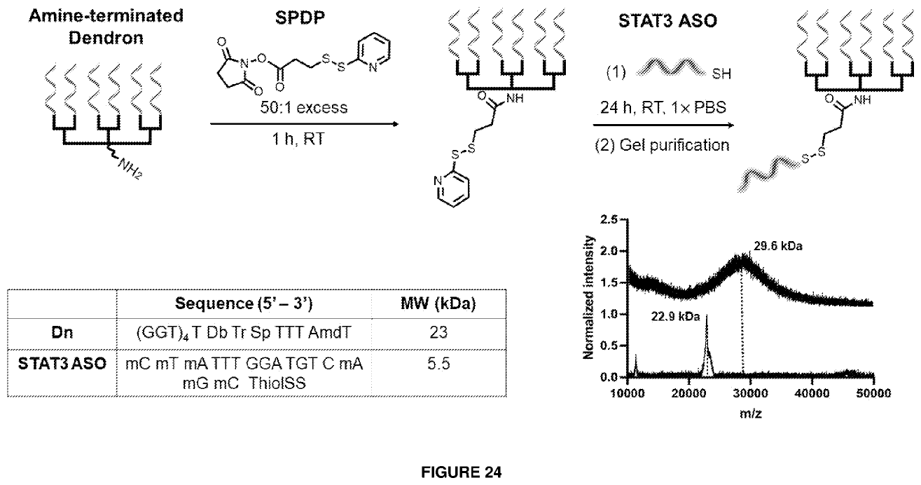

All sequences were synthesized on a MerMade 12 synthesizer (Bio Automation) using conditions recommended by the manufacturer. DNA dendrons containing a primary amine were synthesized on Universal UnyLinker Support CPG beads (2000 Å) with 2× phosphoramidite concentration for bases on the branches of the dendron. STAT3 ASO and control ASO sequences containing a thiol group were synthesized on 3′-Thiol-Modifier C3 S-S CPG beads (1-O-Dimethoxytrityl-propyl-disulfide, 1′-succinyl-Icaa-CPG (1000 Å)). The ASO sequences were purified using RP-HPLC (Agilent), while the DNA dendrons were purified by PAGE. The samples were characterized using MALDI-TOF (AutoFlex-III, Bruker) mass spectrometry (matrix: dihydroxyacetone phosphate). A complete list of synthesized oligonucleotides is shown in Table 3. The concentrations of DNA dendron and ASO were determined by measuring the solution absorbance at λ=260 nm (Cary 5000 UV-vis spectrophotometer, Varian) and using the extinction coefficient calculated by the OligoAnalyzer tool (Integrated DNA Technologies). The amine-containing DNA dendron was reacted with the thiolated ASO sequences via a heterobifunctional crosslinker, succinimidyl 3-(2-pyridyldithio) propionate (SPDP; Thermo Fisher). The conjugate product was purified out by PAGE and characterized by MALDI-TOF. See FIG. 24.

TABLE 3
DNA Design

| | | Calc’d | Exp't | SEQ |
|---|---|---|---|---|
| Name | DNA Sequence (5′-3′) | MW | MW | ID NO: |
| Dn | GGT GGT GGT GGT T D Tr Sp TTT AmdT | 28704.92 | 22947 | 10 |
| STAT3 | mC mT mA TTT GGA TGT C mA mG mC ThiolSS | 5860 | 5470 | 11 |
| ASO | | | | |
| Control | mG mG mC TAC TAC GCC G mT mC mA ThiolSS | 5815 | 5397 | 12 |
| ASO | | | | |

Acronyms for phosphoramidites:
Sp: 18-O-Dimethoxytritylhexaethyleneglycol, 1-[(2-cyanoethyl)-(N,N-diisopropyl)]-phosphoramidite (Spacer phosphoramidite 18)
D: 1,3-bis-[5-(4,4′-dimethoxytrityloxy)pentylamido] propyl-2-[(2-cyanoethyl)-(N,N-diisopropyl)]phosphoramidite (Symmetric doubler phosphoramidite)
Tr: Tris-2,2,2-[3-(4,4′-dimethoxytrityloxy) propyloxymethyl]methyleneoxypropyl-[(2-cyanoethyl)-(N,Ndiisopropyl)]-phosphoramidite (Long trebler phosphoramidite)
AmdT: 5′-Dimethoxytrityl-5-[N-(trifluoroacetylaminohexyl)-3-acrylimido]-2′-deoxyUridin,3′-[(2-cyanoethyl)-(N,N-diisopropyl)]-phosphoramidite (Amino Modifier C6 dT)
mC: 5′-O-(4,4′-Dimethoxytrityl)-2′-O-methoxyethyl-5-methyl-N4-benzoyl-cytidine-3′-O-[(2-cyanoethyl)-(N,N-diisopropyl)]-phosphoramidite (5-Me-C-2′-MOE-phosphoramidite)
mT: 5′-O-(4,4′-Dimethoxytrityl)-2′-O-methoxyethyl-5-methyl-uridine-3′-O-[(2-cyanoethyl)-(N,N-diisopropyl)]-phosphoramidite (5-Me-U-2′-MOE-phosphoramidite).
mA: 5′-O-(4,4′-Dimethoxytrityl)-2′-O-methoxyethyl-N6-benzoyl-adenosine-3′-O-[(2-cyanoethyl)-(N,N-diisopropyl)]-phosphoramidite (A-2′-MOE-phosphoramidite)
mG: 5′-O-(4,4′-Dimethoxytrityl)-2′-O-methoxyethyl-N2-isobutyryl-guanosine-3′-O-[(2-cyanoethyl)-(N,N-diisopropyl)]-phosphoramidite (G-2′-MOE phosphoramidite)
ThiolSS: 3′-Thiol-Modifier C3 S-S(1-O-Dimethoxytrityl-propyl-disulfide, 1′-succinyl-Icaa-CPG (deprotected, unreduced).

### Knockdown Efficiency of STAT3-Targeting ASO Dendrons.

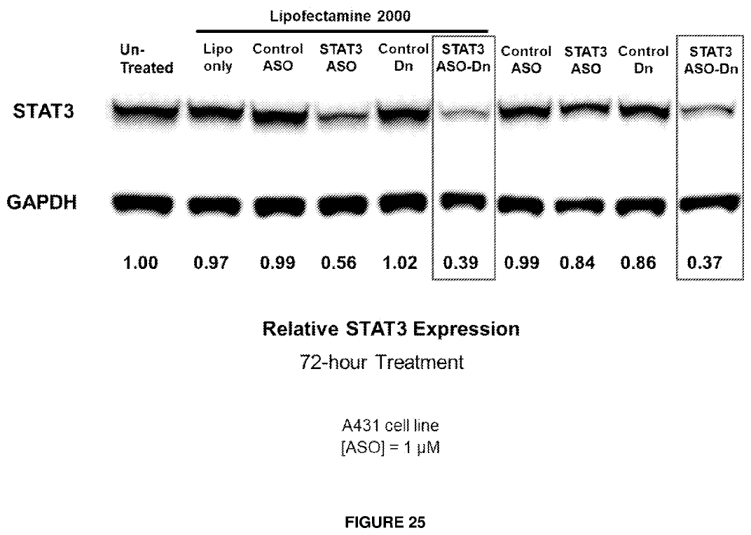

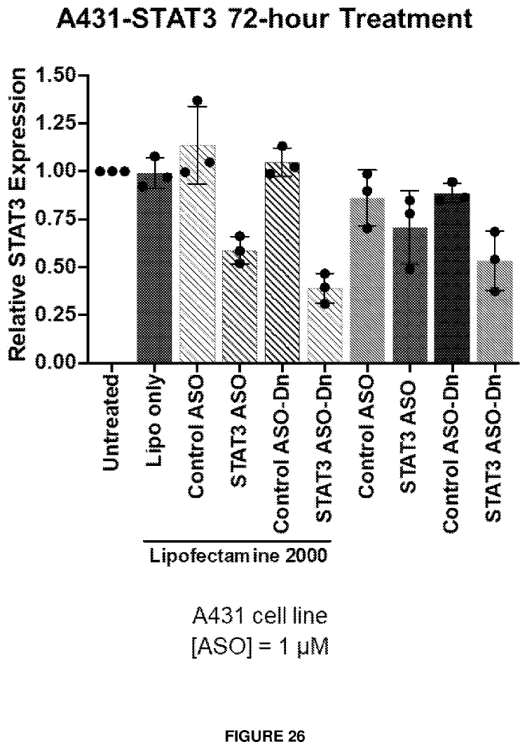

A-431 cells were seeded on collagen I-coated 6-well plates (Corning) at a density of 2× 105/well and incubated for 16 hours prior to treatment. ASO and ASO dendrons were prepared in water and administered to cells at 1 μM. To compare the knockdown efficacy with and without cellular uptake as a variable, treatments were performed both with and without transfection. For transfected groups, Lipofectamine 2000 (Thermo Fisher) was administered under conditions recommended by the manufacturer. After a 72 hour treatment, cell lysates were collected with RIPA Lysis Buffer (Thermo Fisher) containing a phosphatase and protease inhibitor cocktail. A Bradford assay was performed to determine protein concentration in triplicate. The samples were loaded for each treatment at 30 μg per well and separated on bis-Tris 4-12% gradient SDS-PAGE at 100V for 50 minutes. Knockdown of STAT3 protein was determined by Western Blot: The gel was transferred onto a nitrocellulose membrane and incubated with LI-COR blocking buffer for 1 hour at 22° C. The membrane was then incubated with primary antibodies against STAT3 (Cell Signaling #4904) and GAPDH (Cell Signaling #2118) at a 1:2000 dilution for 16 hours at 4° C. The membrane was rinsed three times for 5 min with 1×PBS containing 0.1% Tween 20. To detect primary antibodies, the membrane was incubated with secondary antibody for 1 hour at 22° C. and then rinsed three times for 5 min with 1×PBS containing 0.1% Tween 20. Immune-specific bands were visualized by the Bio-Rad Gel imager (instrument). The relative band signal intensities for STAT3 over GAPDH for each treatment condition were quantified using ImageJ software. See FIG. 25 and FIG. 26.

### REFERENCES

1. Ashley, C. E.; Carnes, E. C.; Phillips, G. K.; Padilla, D.; Durfee, P. N.; Brown, P. A.; Hanna, T. N.; Liu, J.; Phillips, B.; Carter, M. B., The targeted delivery of multicomponent cargos to cancer cells by nanoporous particle-supported lipid bilayers. Nature materials 2011, 10 (5), 389-397.
2. Coulie, P. G.; Van den Eynde, B. J.; Van Der Bruggen, P.; Boon, T., Tumour antigens recognized by T lymphocytes: at the core of cancer immunotherapy. Nature Reviews Cancer 2014, 14 (2), 135-146.
3. Irvine, D. J.; Swartz, M. A.; Szeto, G. L., Engineering synthetic vaccines using cues from natural immunity. Nature materials 2013, 12 (11), 978-990.
4. Khalil, D. N.; Smith, E. L.; Brentjens, R. J.; Wolchok, J. D., The future of cancer treatment: immunomodulation, CARs and combination immunotherapy. Nature reviews Clinical oncology 2016, 13 (5), 273-290.
5. Zhang, L.; Gu, F.; Chan, J.; Wang, A.; Langer, R.; Farokhzad, O., Nanoparticles in medicine: therapeutic applications and developments. Clin. Pharmacol. Ther. 2008, 83 (5), 761-769.
6. Zhang, Y.; Lin, S.; Wang, X.-Y.; Zhu, G., Nanovaccines for cancer immunotherapy. WIREs Nanomedicine and Nanobiotechnology 2019, 11 (5), e1559.
7. Zhu, G.; Zhang, F.; Ni, Q.; Niu, G.; Chen, X., Efficient Nanovaccine Delivery in Cancer Immunotherapy. ACS Nano 2017, 11 (3), 2387-2392.
8. Couvreur, P., Nanoparticles in drug delivery: past, present and future. Adv. Drug Del. Rev. 2013, 65 (1), 21-23.
9. Kemp, J. A.; Shim, M. S.; Heo, C. Y.; Kwon, Y. J., “Combo” nanomedicine: co-delivery of multi-modal therapeutics for efficient, targeted, and safe cancer therapy. Adv. Drug Del. Rev. 2016, 98, 3-18.
10. Wong, K. K.; Li, W. A.; Mooney, D. J.; Dranoff, G., Advances in therapeutic cancer vaccines. Adv. Immunol. 2016, 130, 191-249.
11. Anselmo, A. C.; Mitragotri, S., Nanoparticles in the clinic: An update post COVID-19 vaccines. Bioengineering & translational medicine 2021, 6 (3), e10246.
12. Sallam, M. A.; Prakash, S.; Kumbhojkar, N.; Shields IV, C. W.; Mitragotri, S., Formulation-based approaches for dermal delivery of vaccines and therapeutic nucleic acids: Recent advances and future perspectives. Bioengineering & Translational Medicine 2021, 6 (3), e10215.
13. Sahdev, P.; Ochyl, L. J.; Moon, J. J., Biomaterials for nanoparticle vaccine delivery systems. Pharm. Res. 2014, 31 (10), 2563-2582.
14. Gu, L.; Mooney, D. J., Biomaterials and emerging anticancer therapeutics: engineering the microenvironment. Nature Reviews Cancer 2016, 16 (1), 56-66.
15. Koshy, S. T.; Mooney, D. J., Biomaterials for enhancing anti-cancer immunity. Curr. Opin. Biotechnol. 2016, 40, 1-8.
16. Tzeng, A.; Kauke, M. J.; Zhu, E. F.; Moynihan, K. D.; Opel, C. F.; Yang, N. J.; Mehta, N.; Kelly, R. L.; Szeto, G. L.; Overwijk, W. W., Temporally programmed CD8+DC activation enhances combination cancer immunotherapy. Cell Rep. 2016, 17 (10), 2503-2511.
17. Rincon-Restrepo, M.; Mayer, A.; Hauert, S.; Bonner, D. K.; Phelps, E. A.; Hubbell, J. A.; Swartz, M. A.; Hirosue, S., Vaccine nanocarriers: Coupling intracellular pathways and cellular biodistribution to control CD4 vs CD8 T cell responses. Biomaterials 2017, 132, 48-58.
18. Irvine, D. J.; Aung, A.; Silva, M., Controlling timing and location in vaccines. Adv. Drug Del. Rev. 2020, 158, 91-115.
19. Mehta, N. K.; Pradhan, R. V.; Soleimany, A. P.; Moynihan, K. D.; Rothschilds, A. M.; Momin, N.; Rakhra, K.; Mata-Fink, J.; Bhatia, S. N.; Wittrup, K. D., Pharmacokinetic tuning of protein-antigen fusions enhances the immunogenicity of T-cell vaccines. Nature biomedical engineering 2020, 4 (6), 636-648.
20. Radovic-Moreno, A. F.; Chernyak, N.; Mader, C. C.; Nallagatla, S.; Kang, R. S.; Hao, L.; Walker, D. A.; Halo, T. L.; Merkel, T. J.; Rische, C. H., Immunomodulatory spherical nucleic acids. Proceedings of the National Academy of Sciences 2015, 112 (13), 3892-3897.
21. Skakuj, K.; Wang, S.; Qin, L.; Lee, A.; Zhang, B.; Mirkin, C. A., Conjugation chemistry-dependent T-cell activation with spherical nucleic acids. Journal of the American Chemical Society 2018, 140 (4), 1227-1230.
22. Meckes, B.; Banga, R. J.; Nguyen, S. T.; Mirkin, C. A., Enhancing the Stability and Immunomodulatory Activity of Liposomal Spherical Nucleic Acids through Lipid-Tail DNA Modifications. Small 2018, 14 (5), 1702909.
23. Teplensky, M. H.; Dittmar, J. W.; Qin, L.; Wang, S.; Evangelopoulos, M.; Zhang, B.; Mirkin, C. A., Spherical Nucleic Acid Vaccine Structure Markedly Influences Adaptive Immune Responses of Clinically Utilized Prostate Cancer Targets. Advanced Healthcare Materials 2021, 10 (22), 2101262.
24. Wang, S.; Qin, L.; Yamankurt, G.; Skakuj, K.; Huang, Z.; Chen, P.-C.; Dominguez, D.; Lee, A.; Zhang, B.; Mirkin, C. A., Rational vaccinology with spherical nucleic acids. Proceedings of the National Academy of Sciences 2019, 116 (21), 10473-10481.
25. Teplensky, M. H.; Distler, M. E.; Kusmierz, C. D.; Evangelopoulos, M.; Gula, H.; Elli, D.; Tomatsidou, A.; Nicolaescu, V.; Gelarden, I.; Yeldandi, A.; Batlle, D.; Missiakas, D.; Mirkin, C. A., Spherical nucleic acids as an infectious disease vaccine platform. Proceedings of the National Academy of Sciences 2022, 119 (14), e2119093119.
26. Liu, H.; Irvine, D. J., Guiding principles in the design of molecular bioconjugates for vaccine applications. Bioconjugate chemistry 2015, 26 (5), 791-801.
27. Distler, M. E.; Teplensky, M. H.; Bujold, K. E.; Kusmierz, C. D.; Evangelopoulos, M.; Mirkin, C. A., DNA Dendrons as Agents for Intracellular Delivery. Journal of the American Chemical Society 2021, 143 (34), 13513-13518.
28. Cheng, H. F.; Distler, M. E.; Lee, B.; Zhou, W.; Weigand, S.; Mirkin, C. A., Nanoparticle Superlattices through Template-Encoded DNA Dendrimers. Journal of the American Chemical Society 2021, 143 (41), 17170-17179.
29. Reed, S. G.; Bertholet, S.; Coler, R. N.; Friede, M., New horizons in adjuvants for vaccine development. Trends Immunol. 2009, 30 (1), 23-32.
30. Kawamoto, Y.; Liu, W.; Yum, J. H.; Park, S.; Sugiyama, H.; Takahashi, Y.; Takakura, Y., Enhanced Immunostimulatory Activity of Covalent DNA Dendrons. ChemBioChem 2022, 23 (4), e202100583.
31. Lanier, L. L.; O'Fallon, S.; Somoza, C.; Phillips, J. H.; Linsley, P. S.; Okumura, K.; Ito, D.; Azuma, M., CD80 (B7) and CD86 (B70) provide similar costimulatory signals for T cell proliferation, cytokine production, and generation of CTL. The Journal of Immunology 1995, 154 (1), 97-105.
32. Comberlato, A.; Koga, M. M.; Nussing, S.; Parish, I. A.; Bastings, M. M. C., Spatially Controlled Activation of Toll-like Receptor 9 with DNA-Based Nanomaterials. Nano Lett. 2022, 22 (6), 2506-2513.
33. Chowell, D.; Krishna, S.; Becker, P. D.; Cocita, C.; Shu, J.; Tan, X.; Greenberg, P. D.; Klavinskis, L. S.; Blattman, J. N.; Anderson, K. S., TCR contact residue hydrophobicity is a hallmark of immunogenic CD8+ T cell epitopes. Proceedings of the National Academy of Sciences 2015, 112 (14), E1754-E1762.
34. Fröhlich, E., The role of surface charge in cellular uptake and cytotoxicity of medical nanoparticles. International journal of nanomedicine 2012, 7, 5577.
35. Sainte-Marie, G., The Lymph Node Revisited: Development, Morphology, Functioning, and Role in Triggering Primary Immune Responses. The Anatomical Record 2010, 293 (2), 320-337.
36. Ali Khan, A.; Mudassir, J.; Mohtar, N.; Darwis, Y., Advanced drug delivery to the lymphatic system: lipid-based nanoformulations. Int J Nanomedicine 2013, 8, 2733-44.
37. Hawley, A. E.; Davis, S. S.; Illum, L., Targeting of colloids to lymph nodes: influence of lymphatic physiology and colloidal characteristics. Adv. Drug Del. Rev. 1995, 17 (1), 129-148.
38. Liu, J.; Wong, H.-L.; Moselhy, J.; Bowen, B.; Wu, X. Y.; Johnston, M. R., Targeting colloidal particulates to thoracic lymph nodes. Lung Cancer 2006, 51 (3), 377-386.
39. S. H. Kim, T.-H. Lee, Conformational Dynamics of Poly (T) Single-Stranded DNA at the Single-Molecule Level. The Journal of Physical Chemistry Letters 12, 4576-4584 (2021).
40. X. Liang, H. Kuhn, M. D. Frank-Kamenetskii, Monitoring single-stranded DNA secondary structure formation by determining the topological state of DNA catenanes. Biophysical journal 90, 2877-2889 (2006).
41. R. Bar-Ziv, A. Libchaber, Effects of DNA sequence and structure on binding of RecA to single-stranded DNA. Proceedings of the National Academy of Sciences 98, 9068-9073 (2001).

What is claimed is:

1. An oligonucleotide dendron comprising an oligonucleotide stem linked to a plurality of oligonucleotide branches through one or more of a doubler moiety, a trebler moiety, or a combination thereof, wherein one or more of the plurality of oligonucleotide branches comprises an additional agent, and wherein the additional agent is conjugated at an internal location that is anywhere along the length of the one or more of the plurality of oligonucleotide branches that is not an end of the one or more of the plurality of oligonucleotide branches.

2. An oligonucleotide dendron comprising an oligonucleotide stem linked to a plurality of oligonucleotide branches through one or more of a doubler moiety, a trebler moiety, or a combination thereof, wherein a free end of the one or more of the plurality of oligonucleotide branches is conjugated to an additional agent.

3. The oligonucleotide dendron of claim 1 or claim 2, wherein all of the plurality of oligonucleotide branches comprises an additional agent.

4. The oligonucleotide dendron of any one of claims 1-3, wherein the oligonucleotide dendron is a DNA dendron, a RNA dendron, a modified oligonucleotide dendron, or a combination thereof.

5. The oligonucleotide dendron of any one of claims 1-4, wherein the oligonucleotide dendron comprises about 2 to about 27 oligonucleotide branches.

6. The oligonucleotide dendron of any one of claims 1-5, wherein the oligonucleotide dendron comprises 6 oligonucleotide branches.

7. The oligonucleotide dendron of any one of claims 1-5, wherein the oligonucleotide dendron comprises 9 oligonucleotide branches.

8. The oligonucleotide dendron of any one of claims 1-7, wherein the oligonucleotide stem comprises a homopolymeric nucleotide sequence.

9. The oligonucleotide dendron of any one of claims 1-7, wherein the oligonucleotide stem comprises an immunostimulatory oligonucleotide.

10. The oligonucleotide dendron of any one of claims 1-9, wherein one or more of the plurality of oligonucleotide branches comprises an immunostimulatory oligonucleotide.

11. The oligonucleotide dendron of any one of claims 1-10, wherein all of the plurality of oligonucleotide branches comprises an immunostimulatory oligonucleotide.

12. The oligonucleotide dendron of any one of claims 1-11, wherein one or more of the plurality of oligonucleotide branches comprises a homopolymeric nucleotide sequence.

13. The oligonucleotide dendron of any one of claims 1-12, wherein all of the plurality of oligonucleotide branches comprises a homopolymeric nucleotide sequence.

14. The oligonucleotide dendron of any one of claims 9-13, wherein the immunostimulatory oligonucleotide is a toll-like receptor (TLR) agonist.

15. The oligonucleotide dendron of claim 14, wherein the TLR agonist is a toll-like receptor 1 (TLR-1) agonist, toll-like receptor 2 (TLR-2) agonist, toll-like receptor 3 (TLR-3) agonist, toll-like receptor 4 (TLR-4) agonist, toll-like receptor 5 (TLR-5) agonist, toll-like receptor 6 (TLR-6) agonist, toll-like receptor 7 (TLR-7) agonist, toll-like receptor 8 (TLR-8) agonist, toll-like receptor 9 (TLR-9) agonist, toll-like receptor 10 (TLR-10) agonist, toll-like receptor 11 (TLR-11) agonist, toll-like receptor 12 (TLR-12) agonist, toll-like receptor 13 (TLR-13) agonist, or a combination thereof.

16. The oligonucleotide dendron of any one of claims 1-15, wherein the oligonucleotide stem comprises a toll-like receptor (TLR) antagonist.

17. The oligonucleotide dendron of any one of claims 1-16, wherein one or more of the plurality of oligonucleotide branches comprises a toll-like receptor (TLR) antagonist.

18. The oligonucleotide dendron of any one of claims 1-17, wherein all of the plurality of oligonucleotide branches comprises a toll-like receptor (TLR) antagonist.

19. The oligonucleotide dendron of any one of claims 16-18, wherein the TLR-antagonist is a toll-like receptor 1 (TLR-1) antagonist, toll-like receptor 2 (TLR-2) antagonist, toll-like receptor 3 (TLR-3) antagonist, toll-like receptor 4 (TLR-4) antagonist, toll-like receptor 5 (TLR-5) antagonist, toll-like receptor 6 (TLR-6) antagonist, toll-like receptor 7 (TLR-7) antagonist, toll-like receptor 8 (TLR-8) antagonist, toll-like receptor 9 (TLR-9) antagonist, toll-like receptor 10 (TLR-10) antagonist, toll-like receptor 11 (TLR-11) antagonist, toll-like receptor 12 (TLR-12) antagonist, toll-like receptor 13 (TLR-13) antagonist, or a combination thereof.

20. The oligonucleotide dendron of any one of claims 1-19, wherein one or more of the plurality of oligonucleotide branches are double stranded.

21. The oligonucleotide dendron of any one of claims 1-20, wherein all of the plurality of oligonucleotide branches are double stranded.

22. The oligonucleotide dendron of any one of claims 1-21, wherein one or more of the plurality of oligonucleotide branches are single stranded.

23. The oligonucleotide dendron of any one of claims 1-22, wherein all of the plurality of oligonucleotide branches are single stranded.

24. The oligonucleotide dendron of any one of claims 1-23, wherein the oligonucleotide stem is double stranded.

25. The oligonucleotide dendron of any one of claims 1-24, wherein the oligonucleotide stem is single stranded.

26. The oligonucleotide dendron of any one of claims 1-25, wherein the oligonucleotide dendron comprises 1, 2, 3, 4, 5, 6, 7, 8, 9, 10, 11, 12, 13, 14, 15, 16, 17, 18, 19, 20, 21, 22, 23, 24, 25, 26, 27 or more additional agents.

27. The oligonucleotide dendron of any one of claims 1-26, wherein the additional agent is a protein, a peptide, or a combination thereof.

28. The oligonucleotide dendron of any one of claims 1-27, wherein the additional agent is an antigen.

29. The oligonucleotide dendron of claim 28, wherein the antigen is a viral antigen, a cancer-related antigen, or a combination thereof.

30. The oligonucleotide dendron of any one of claims 1-29, wherein the oligonucleotide stem comprises an inhibitory oligonucleotide.

31. The oligonucleotide dendron of any one of claims 1-30, wherein one or more of the plurality of oligonucleotide branches comprises an inhibitory oligonucleotide.

32. The oligonucleotide dendron of any one of claims 1-31, wherein all of the plurality of oligonucleotide branches comprises an inhibitory oligonucleotide.

33. The oligonucleotide dendron of any one of claims 30-32, wherein the inhibitory oligonucleotide is antisense DNA, small interfering RNA (siRNA), an aptamer, a short hairpin RNA (shRNA), a DNAzyme, or an aptazyme.

34. The oligonucleotide dendron of any one of claims 1-33, wherein the doubler moiety comprises a structure prior to incorporation into the oligonucleotide dendron that is: 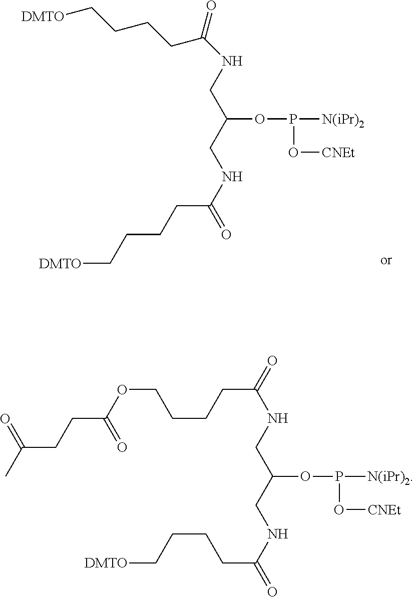
DMT=4,4′-dimethoxytriryl; O=oxygen; C═carbon; N=nitrogen; Et=ethyl; iPr=isopropyl.

35. The oligonucleotide dendron of any one of claims 1-34, wherein the trebler moiety comprises a structure prior to incorporation into the oligonucleotide dendron that is: 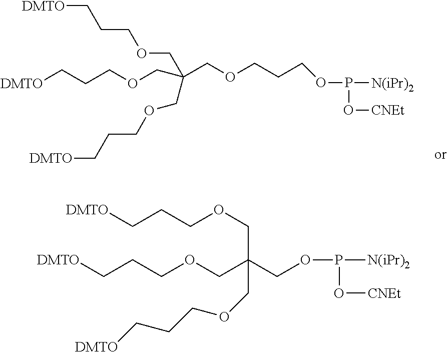
DMT=4,4′-dimethoxytriryl; O-oxygen; C═carbon; N=nitrogen; Et=ethyl; iPr=isopropyl

36. An oligonucleotide dendron comprising an oligonucleotide stem linked to a plurality of oligonucleotide branches through one or more of a doubler moiety, a trebler moiety, or a combination thereof, wherein:
(A) the oligonucleotide stem comprises an inhibitory oligonucleotide and is single-stranded, and
(B) one or more of the plurality of oligonucleotide branches are single stranded.

37. The oligonucleotide dendron of claim 36, wherein all of the plurality of oligonucleotide branches are single stranded.

38. The oligonucleotide dendron of claim 36 or claim 37, wherein the inhibitory oligonucleotide is antisense DNA, small interfering RNA (siRNA), an aptamer, a short hairpin RNA (shRNA), a DNAzyme, or an aptazyme.

39. The oligonucleotide dendron of any one of claims 36-38, wherein one or more or all of the plurality of oligonucleotide branches comprises a homopolymeric nucleotide sequence.

40. The oligonucleotide dendron of any one of claims 36-39, wherein all of the plurality of oligonucleotide branches comprises a homopolymeric nucleotide sequence.

41. The oligonucleotide dendron of any one of claims 36-40, wherein the oligonucleotide dendron is a DNA dendron, a RNA dendron, a modified oligonucleotide dendron, or a combination thereof.

42. The oligonucleotide dendron of any one of claims 36-41, wherein the oligonucleotide dendron comprises about 2 to about 27 oligonucleotide branches.

43. The oligonucleotide dendron of any one of claims 36-42, wherein the oligonucleotide dendron comprises 6 oligonucleotide branches.

44. The oligonucleotide dendron of any one of claims 36-42, wherein the oligonucleotide dendron comprises 9 oligonucleotide branches.

45. The oligonucleotide dendron of any one of claims 36-44, wherein the doubler moiety comprises a structure prior to incorporation into the oligonucleotide dendron that is: 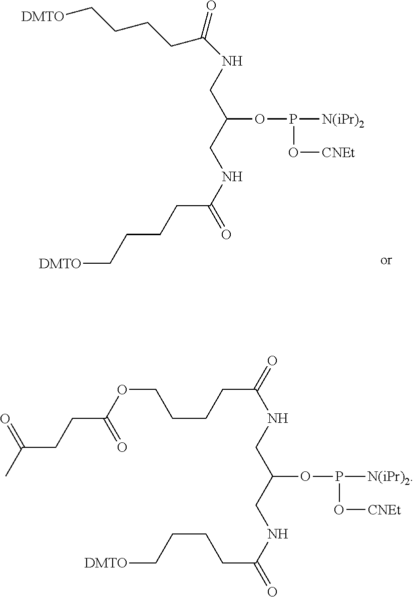
DMT=4,4′-dimethoxytriryl; O=oxygen; C═carbon; N=nitrogen; Et=ethyl; iPr=isopropyl.

46. The oligonucleotide dendron of any one of claims 36-45, wherein the trebler moiety comprises a structure prior to incorporation into the oligonucleotide dendron that is: 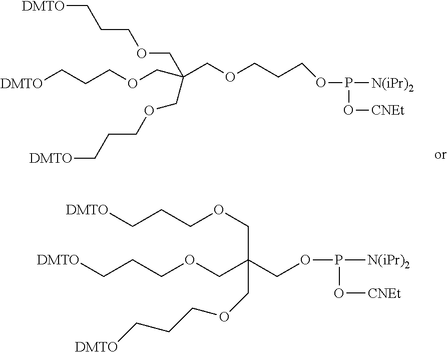
DMT=4,4′-dimethoxytriryl; O=oxygen; C═carbon; N=nitrogen; Et=ethyl; iPr=isopropyl

47. A pharmaceutical formulation comprising a plurality of the oligonucleotide dendrons of any one of claims 1-46 and a pharmaceutically acceptable carrier or diluent.

48. A vaccine comprising the oligonucleotide dendron of any one of claims 1-46 or the pharmaceutical formulation of claim 47.

49. The vaccine of claim 48, comprising an adjuvant.

50. An antigenic composition comprising the oligonucleotide dendron of any one of claims 1-46 in a pharmaceutically acceptable carrier, diluent, stabilizer, preservative, or adjuvant, the pharmaceutical formulation of claim 47, or the vaccine of claim 48 or 49, wherein the antigenic composition is capable of generating an immune response including antibody generation, an antitumor response, an antiviral response, and/or a protective immune response in a mammalian subject.

51. The antigenic composition of claim 50, wherein the antibody response is a neutralizing antibody response or a protective antibody response.

52. A method of producing an immune response in a subject in need thereof, comprising administering to the subject an effective amount of the oligonucleotide dendron of any one of claims 1-46, the pharmaceutical formulation of claim 47, the vaccine of claim 48 or claim 49, or the antigenic composition of claim 50 or claim 51, thereby producing the immune response in the subject.

53. The method of claim 52, wherein the subject has cancer or is at risk of developing cancer, or the subject has a viral infection or is at risk of developing a viral infection, or a combination thereof.

54. The method of claim 53, wherein the cancer is breast cancer, peritoneum cancer, cervical cancer, colon cancer, rectal cancer, esophageal cancer, eye cancer, liver cancer, pancreatic cancer, larynx cancer, lung cancer, skin cancer, ovarian cancer, prostate cancer, stomach cancer, testicular cancer, thyroid cancer, brain cancer, or a combination thereof.

55. The method of any one of claims 52-54, wherein the viral infection is due to a Coronaviridae virus, an Arteriviridae virus, a Roniviridae virus, a Picornaviridae virus, or a combination thereof.

56. The method of claim 55, wherein the viral infection is due to Coronavirus, MERS, alphacoronavirus HCoV-NL63, betacoronaviruses HCoV-OC43, H1N1 influenza A, influenza BSARS, or a combination thereof.

57. The method of claim 56, wherein the Coronavirus is SARS-COV-2 and/or a variant thereof.

58. A method of treating a cancer in a subject in need thereof, comprising administering to the subject an effective amount of the oligonucleotide dendron of any one of claims 1-46, the pharmaceutical formulation of claim 47, the vaccine of claim 48 or claim 49, or the antigenic composition of claim 50 or claim 51, thereby treating the cancer in the subject.

59. The method of claim 58, wherein the cancer is breast cancer, peritoneum cancer, cervical cancer, colon cancer, rectal cancer, esophageal cancer, eye cancer, liver cancer, pancreatic cancer, larynx cancer, lung cancer, skin cancer, ovarian cancer, prostate cancer, stomach cancer, testicular cancer, thyroid cancer, brain cancer, or a combination thereof.

60. A method of treating a viral infection in a subject in need thereof, comprising administering to the subject an effective amount of the oligonucleotide dendron of any one of claims 1-46, the pharmaceutical formulation of claim 47, the vaccine of claim 48 or claim 49, or the antigenic composition of claim 50 or claim 51, thereby treating the viral infection in the subject.

61. The method of claim 60, wherein the viral infection is due to a Coronaviridae virus, an Arteriviridae virus, a Roniviridae virus, a Picornaviridae virus, or a combination thereof.

62. The method of claim 61, wherein the viral infection is due to Coronavirus, MERS, alphacoronavirus HCoV-NL63, betacoronaviruses HCoV-OC43, H1N1 influenza A, influenza BSARS, or a combination thereof.

63. The method of claim 62, wherein the Coronavirus is SARS-COV-2 and/or a variant thereof.

64. The method of any one of claims 58-63, wherein the administering is subcutaneous.

65. The method of any one of claims 58-63, wherein the administering is intravenous, intraperitoneal, intranasal, or intramuscular.

66. A composition comprising a plurality of the oligonucleotide dendrons of any one of claims 1-46.

67. A method of inhibiting expression of a gene comprising the step of hybridizing a polynucleotide encoding the gene product with the oligonucleotide dendron of any one of claims 1-46, or the composition of claim 66, wherein hybridizing between the polynucleotide and the oligonucleotide stem and/or one or more or all of the plurality of oligonucleotide branches of the oligonucleotide dendron occurs over a length of the polynucleotide with a degree of complementarity sufficient to inhibit expression of the gene product.

68. The method of claim 67, wherein hybridizing between the polynucleotide and the oligonucleotide stem and/or all of the plurality of oligonucleotide branches of the oligonucleotide dendron occurs over a length of the polynucleotide with a degree of complementarity sufficient to inhibit expression of the gene product.

69. The method of claim 67 or claim 68, wherein the hybridizing between the polynucleotide and the oligonucleotide stem and/or one or more of the plurality of oligonucleotide branches of the oligonucleotide dendron occurs in the absence of a transfection reagent.

70. The method of any one of claims 67-69, wherein the hybridizing between the polynucleotide and the oligonucleotide stem and/or all of the plurality of oligonucleotide branches of the oligonucleotide dendron occurs in the absence of a transfection reagent.

71. A method for up-regulating activity of a toll-like receptor (TLR), comprising contacting a cell having the toll-like receptor with the oligonucleotide dendron of any one of claims 1-46 or the composition of claim 66.

72. A method for down-regulating activity of a toll-like receptor (TLR), comprising contacting a cell having the toll-like receptor with the oligonucleotide dendron of any one of claims 1-46 or the composition of claim 66.


SEQUENCE LISTING (12 sequences)

SEQ ID NO: 1 - DNA, 20 bp, synthetic construct
  source 1..20: mol_type = other DNA
tccatgacgttcctgacgtt

SEQ ID NO: 2 - DNA, 30 bp, synthetic construct
  source 1..30: mol_type = other DNA
tttttttttttccatgacgttcctgacgtt

SEQ ID NO: 3 - DNA, 30 bp, synthetic construct
  source 1..30: mol_type = other DNA
ttttttttttaacgtcaggaacgtcatgga

SEQ ID NO: 4 - DNA, 30 bp, synthetic construct
  source 1..30: mol_type = other DNA
tccatgacgttcctgacgtttttttttttt

SEQ ID NO: 5 - DNA, 30 bp, synthetic construct
  source 1..30: mol_type = other DNA
aacgtcaggaacgtcatggatttttttttt

SEQ ID NO: 6 - DNA, 30 bp, synthetic construct; identical to SEQ ID NO: 2
  source 1..30: mol_type = other DNA
  modified_base 30: mod_base = OTHER; note = 5'-Dimethoxytrityl-5-[N-(trifluoroacetylaminohexyl) -3-acrylimido]-2'-deoxyUridine,3'-[(2-cyanoethyl)-(N,N-dii sopropyl)]-phosphoramidite (Amino Modifier C6 dT)

SEQ ID NO: 7 - DNA, 30 bp, synthetic construct; identical to SEQ ID NO: 2
  source 1..30: mol_type = other DNA
  modified_base 6: mod_base = OTHER; note = 5'-Dimethoxytrityl-5-[N-(trifluoroacetylaminohexyl) -3-acrylimido]-2'-deoxyUridine,3'-[(2-cyanoethyl)-(N,N-dii sopropyl)]-phosphoramidite (Amino Modifier C6 dT)

SEQ ID NO: 8 - DNA, 30 bp, synthetic construct; identical to SEQ ID NO: 2
  source 1..30: mol_type = other DNA
  modified_base 1: mod_base = OTHER; note = 5'-Dimethoxytrityl-5-[N-(trifluoroacetylaminohexyl) -3-acrylimido]-2'-deoxyUridine,3'-[(2-cyanoethyl)-(N,N-dii sopropyl)]-phosphoramidite (Amino Modifier C6 dT)

SEQ ID NO: 9 - protein, 10 aa, synthetic construct
VYDFAFRDLC

SEQ ID NO: 10 - DNA, 17 bp, synthetic construct
  source 1..17: mol_type = other DNA
  modified_base 17: mod_base = OTHER; note = 5'-Dimethoxytrityl-5-[N-(trifluoroacetylaminohexyl) -3-acrylimido]-2'-deoxyUridin,3'-[(2-cyanoethyl)-(N,N-diis opropyl)]-phosphoramidite (Amino Modifier C6 dT)
ggtggtggtggtttttt

SEQ ID NO: 11 - DNA, 16 bp, synthetic construct
  source 1..16: mol_type = other DNA
  modified_base 1: mod_base = OTHER; note = 5'-O-(4,4'-Dimethoxytrityl)-2'-O-methoxyethyl-5-met hyl-N4-benzoyl- cytidine-3'-O-[(2-cyanoethyl)-(N,N-diisopr opyl)]-phosphoramidite (5-Me-C-2'-MOE-phosphoramidite)
  modified_base 2: mod_base = OTHER; note = 5'-O-(4,4'-Dimethoxytrityl)-2'-O-methoxyethyl-5-met hyl-uridine-3'-O-[(2-cyanoethyl)-(N,N-diisopropyl)]-phosph oramidite (5-Me-U-2'-MOE-phosphoramidite)
  modified_base 3: mod_base = OTHER; note = 5'-O-(4,4'-Dimethoxytrityl)-2'-O-methoxyethyl-N6-be nzoyl-adenosine -3'-O-[(2-cyanoethyl)-(N,N-diisopropyl)]-phosphoramidite (A-2'-MOE-phosphoramidite)
  modified_base 14: mod_base = OTHER; note = 5'-O-(4,4'-Dimethoxytrityl)-2'-O-methoxyethyl-N6-be nzoyl-adenosine -3'-O-[(2-cyanoethyl)-(N,N-diisopropyl)]-phosphoramidite (A-2'-MOE-phosphoramidite)
  modified_base 15: mod_base = OTHER; note = 5'-O-(4,4'-Dimethoxytrityl)-2'-O-methoxyethyl-N2-is obutyryl- guanosine-3'-O-[(2-cyanoethyl)-(N,N-diisopropyl) ]-phosphoramidite (G-2'-MOE phosphoramidite)
  modified_base 16: mod_base = OTHER; note = 5'-O-(4,4'-Dimethoxytrityl)-2'-O-methoxyethyl-5-met hyl-N4-benzoyl- cytidine-3'-O-[(2-cyanoethyl)-(N,N-diisopr opyl)]-phosphoramidite (5-Me-C-2'-MOE-phosphoramidite)
ctatttggatgtcagc

SEQ ID NO: 12 - DNA, 16 bp, synthetic construct
  source 1..16: mol_type = other DNA
  modified_base 1: mod_base = OTHER; note = 5'-O-(4,4'-Dimethoxytrityl)-2'-O-methoxyethyl-N2-is obutyryl- guanosine-3'-O-[(2-cyanoethyl)-(N,N-diisopropyl) ]-phosphoramidite (G-2'-MOE phosphoramidite)
  modified_base 2: mod_base = OTHER; note = 5'-O-(4,4'-Dimethoxytrityl)-2'-O-methoxyethyl-N2-is obutyryl- guanosine-3'-O-[(2-cyanoethyl)-(N,N-diisopropyl) ]-phosphoramidite (G-2'-MOE phosphoramidite)
  modified_base 3: mod_base = OTHER; note = 5'-O-(4,4'-Dimethoxytrityl)-2'-O-methoxyethyl-5-met hyl-N4-benzoyl- cytidine-3'-O-[(2-cyanoethyl)-(N,N-diisopr opyl)]-phosphoramidite (5-Me-C-2'-MOE-phosphoramidite)
  modified_base 14: mod_base = OTHER; note = 5'-O-(4,4'-Dimethoxytrityl)-2'-O-methoxyethyl-5-met hyl-uridine-3'-O-[(2-cyanoethyl)-(N,N-diisopropyl)]-phosph oramidite (5-Me-U-2'-MOE-phosphoramidite)
  modified_base 15: mod_base = OTHER; note = 5'-O-(4,4'-Dimethoxytrityl)-2'-O-methoxyethyl-5-met hyl-N4-benzoyl- cytidine-3'-O-[(2-cyanoethyl)-(N,N-diisopr opyl)]-phosphoramidite (5-Me-C-2'-MOE-phosphoramidite)
  modified_base 16: mod_base = OTHER; note = 5'-O-(4,4'-Dimethoxytrityl)-2'-O-methoxyethyl-N6-be nzoyl-adenosine -3'-O-[(2-cyanoethyl)-(N,N-diisopropyl)]-phosphoramidite (A-2'-MOE-phosphoramidite)
ggctactacgccgtca